(19) United States
(12) Patent Application Publication
(10) Pub. No.: US 2026/0125357 A1
(43) Pub. Date: May 7, 2026

(54) LACTATE ENHANCING COMPOUNDS AND USES THEREOF

(71) Applicant: GLIAPHARM SA, GENÈVE (CH)

(72) Inventors: CHARLES FINSTERWALD, CRASSIER (CH); SYLVAIN LENGACHER, LAUSANNE 26 (CH); PIERRE MAGISTRETTI, CRANS-MONTANA (CH); TIMOTHY RITCHIE, ST ALBANS (GB); HANS MAAG, OBERAMMERGAU (DE); GUILLAUME DUVEY, THYEZ (FR)

(21) Appl. No.: 19/117,674

(22) PCT Filed: Oct. 2, 2023
(86) PCT No.: PCT/EP2023/077192

§ 371 Date: Apr. 2, 2025

(30) Foreign Application Priority Data
Oct. 3, 2022 (EP) 22199427.0

(51) Int. Cl. C07D 401/04; A61K 31/4725; A61K 31/506; A61P 25/00; A61P 25/28; C07D 217/02; C07D 217/22; C07D 401/10; C07D 401/12; C07D 405/12; C07D 413/12

(52) U.S. Cl. CPC C07D 401/04; A61K 31/4725; A61K 31/506; A61P 25/00; A61P 25/28; C07D 217/02; C07D 217/22; C07D 401/10; C07D 401/12; C07D 405/12; C07D 413/12

(57) ABSTRACT

The present invention relates to new agents useful for stimulating uptake of glucose and release of lactate by astrocytes. The invention further relates to methods of preparation, formulations and therapeutic uses of those agents, notably for the prevention and/or treatment of neurological disorders with brain energy hypometabolism, in particular neurodegenerative, neurometabolic and psychiatric disorders or improving cognitive and memory functions.

### FIELD OF THE INVENTION

The present invention relates generally to the field of lactate enhancing agents and in particular the use of lactate enhancing agents for the treatment of neurological disorders, comprising neurodegenerative and psychiatric diseases.

### BACKGROUND OF THE INVENTION

Neurological disorders represent some of the pathologies with the highest unmet needs. They are expected to become the first cause of death by 2050. These pathologies are complex and difficult to treat. Finding treatments has been a challenge for the pharmaceutical industry for the past decades, but limited progress has been made in the field. Most of the therapeutic strategies so far have aimed at targeting neurons directly, using a ‘neuro-centric’ approach, largely letting aside the important role of other cell types of the nervous system, including astrocytes.

Astrocytes outnumber neurons in the brain and together with oligodendrocytes and microglia form a category of cells called glial cells that support neuronal activity and survival. In the past decade, considerable attention has been focused on understanding the role of astrocytes in physiological processes, as well as their implication in the development of neurological diseases including neurodegenerative disorders, age-related cognitive impairments and psychiatric diseases. While glial cells were though for a long time to only be important for nervous tissue structural support—a sort of brain ‘glue’—, their much more important role for the control of fundamental processes has now been largely acknowledged. In particular, astrocytes play a fundamental role by providing energy to neurons, which is required for their function—transmit electrical information—and survival. Hence, astrocytes were found to be key for numerous brain physiological processes that include neuronal protection, neuronal function, synaptic plasticity, and memory consolidation (Magistretti et al., 2018, Nat. Rev. Neurosci., 19(4):235-249). Although neurological disorders have historically been considered as pathologies that exclusively result from neuronal dysfunction and death, it has become clear that other cell types, such as astrocytes, contribute to these pathologies. A large body of evidence has linked astrocytes activity to mild cognitive impairments (MCI), Alzheimer's disease (AD), amyotrophic lateral sclerosis (ALS), depression and others. For instance, in AD patients, activated astrocytes are preferentially located in the vicinity of amyloid plaques, where they exhibit abnormal morphology and mitochondrial function. In the early stage of the disease, activated astrocytes have neuroprotective action by internalizing and degrading amyloid plaques, while upon progression of the disease, deposit of amyloid plaques leads to astrocytic death that in turn results in further amyloid accumulation (Nagele et al. 2004, Neurobiol. Aging, 25(5):663-74). There are clear indications that age-related changes of astrocytes have a role in the development of age-related neurodegenerative disorders, such as MCI and AD (Cai et al., 2017, J. Neurol. 264(10):2068-74). Astrocytosis is a typical morphological feature of the AD brain and represents either proliferation of astrocytes, in an effort to support dying neurons, or a reaction to degrade the increasing amounts of toxic β-amyloid peptides. Of particular interest, exposure of astrocytes to β Amyloid in vitro alters their metabolic activity thus resulting in reduction of the neuronal protection against oxidative stresses (Allaman et al., 2010, J. Neurosci 30(9):3326-38).

Deregulation of brain energy metabolism is an important contributor to the development of several neurological disorders and age-associated cognitive decline. These disorders have been linked to decreased mitochondrial activity, increased oxidative stress and diminished cerebral glucose metabolism. For instance, glucose hypometabolism in the brain appears early in the genesis of AD and in fact represents a common phenomenon with other neurodegenerative diseases (Yin et al, 2016, Free Radic. Biol. Med., 100:108-22; Fu et al. 2014, Biogerontology, 15(6):579-86; Demetrius et al, 2013, Biogerontology, 14(6):641-9; Demetrius et al., 2014, front Physiol 5:522; Tomi et al., 2013, Brain Res., 1495:61-75; Ferreira et al., 2010, Curr Drug Targets, 11(10):1193-2016). Mitochondrial dysfunction, which is associated with age-related neurodegeneration, is particularly prevalent in AD (Beal, 2005, Neurobiol Aging, 26(5):585-6; Yao et al., 2011, Curr Pharm Des 17(31):3474-9). Studies on patients with AD and mouse models have highlighted the down regulation of several cerebral genes involved in energy regulation (Liang et al., 2008, Proc. Natl. Acad. Sci. USA, March 18; 105(11):4441-6). As a result, significant correlation between diminished cerebral glucose metabolism and cognitive performance has been shown in AD patients (Thomas et al., 2015, J. Nutr. Health Aging, 19(1):58-63; Woo et al, 2010, Int. J. Geriatr. Psychiatry, 25(11):1150-8). Together, these data indicate that astrocytes activity and metabolic coupling that are impaired in MCI and AD may result in the characteristic accumulation of amyloid plaques and neuronal degeneration in specific brain areas.

In ALS, deficit of mitochondrial activity and energy production were found to be responsible, at least in part, for motor neuron degeneration (Boillee et al., 2006, Neuron, 52:39-59). Astrocytes may also play an important role through the regulation of glutamate uptake, which is dramatically impaired in ALS (Rothstein et al., 1990, Ann. Neurol., 28:18-25; Spreux-Varoquaux et al., 2002, J. Neurol. Sci., 193:73-78).

Other neurological disorders including psychiatric disorders such as depression also exhibit impairments in brain energy metabolism (Elsayed and Magistretti, 2015, Front Cell Neurosci, 9:468). In addition, growing evidence indicates that alterations of glial cells also contribute to the pathophysiology and treatment of major depression (Rajkowska and Stockmeier, 2013, Curr Drug Targets 14, 1225-1236; Czéh et al., 2006, Neuropsychopharmacology 31, 1616-1626; Banasr et al., 2010, Mol Psychiatry 15, 501-511; Banasr and Duman, 2008, Biol Psychiatry 64, 863-870).

Other neurological disorders exhibit prototypic brain energy hypometabolism, such as glucose transporter 1-deficiency syndrome (GLUT1-DS), also known as De Vivo disease. GLUT1-DS is a genetic disease caused by a mutation in the GLUT1 gene, also known as solute carrier family 2, facilitated glucose transporter member 1 (SLC2A1). Patients carrying hemizygosity of GLUT1 and nonsense mutations resulting in truncation of the GLUT1 protein have normal circulating blood glucose, but low cerebrospinal fluid (CSF) lactate, persistent hypoglycorrhachia (low CSF glucose) and diminished transport of hexose into isolated red blood cells (De ViVo et al., 1991, N. Engl. J. Med., 325, 703-709). Symptoms of GLUT1-DS can vary in severity depending on the mutation in SLC2A1 gene. They include, without limitation, mental retardation, cognitive impairment, epilepsy and motor function problems (including ataxia, gait disturbance, dystonia, dysarthria, aberrant gaze saccades, spasticity, and other paroxysmal neurologic phenomena) (Gras et al, 2014, Revue Neurologoqique 170:91-99).

Interestingly, a specific metabolite of glucose, i.e. lactate, appears to play a particularly important role in astrocyte-neuron metabolic coupling. Indeed, lactate that is produced by astrocytes is used by neurons as preferential energy source upon neuronal activity, through the so-called astrocyte-neuron lactate shuttle (ANLS) (Pellerin et al., 2012,J, Cereb Blood Flow Metab., 32(7):1152-66). Lactate is produced in astrocytes through the process of aerobic glycolysis, i.e. the transformation of glucose into lactate in the presence of oxygen, a process usually better known to occur in the absence of oxygen to produce energy (e.g. in muscles during physical activity). The source of glucose in the brain can either come from the circulation (astrocyte endfeet are in close contact with capillaries) or from internal stores of glycogen (cerebral glycogen is exclusively present in astrocytes). Upon synaptic activity, lactate is produced by astrocytes and transferred to neurons, where it is transformed into pyruvate to enter the Tricarboxylic acid (TCA) cycle and produce ATP. In this context, lactate was shown to act as a neuroprotective agent against glutamate-mediated excitotoxicity (Jourdain et al., 2016, Sci. Rep., 6:21250), as well as against cerebral ischemia in vivo (Berthet et al., 2012, Cerebrovasc Dis., 34(5-6):329-35). In addition to its neuroprotective effects, ANLS was found to be key in the regulation of long-term memory consolidation (Suzuki et al, 2011, Cell 144(5):810-23), as well as in the regulation of the expression of genes that modulate synaptic function and plasticity (Yang et al., 2014, Proc Natl Acad Sci USA, 111(33):12228-33; Tadi et al. 2015, PLoS One, 10(10):e0141568). Lactate indeed not only plays a key role in providing energy to neurons, but also acts as a regulator of synaptic plasticity through signalling activities (Magistretti and Allaman, 2018, Nat Rev Neurosci 19(4):235-249). Furthermore, the transport of lactate was found to be impaired in a mouse model of ALS, as well as in the nervous system of ALS patients (Lee et al., 2012, Nature, 487(7408):443-8), providing additional evidence for the role of lactate in neurodegenerative disease. In addition, administration of lactate was found to produce antidepressant like effects in a number of animal models of depression (Carrard et al., 2018, Mol Psychiatry, 23(2):488). Based on these recent studies, lactate, previously misunderstood as a waste by-product of glycolysis in the past, has been proposed as being a key signal molecule that regulates the beneficial adaptation of the brain caused by exercise and it was proposed a central protective mechanism may underlie the cognitive benefits induced by exercise. For people who cannot enjoy the benefits of this higher-intensity exercise due to physical reasons, it is believed that lactate enhancement could partially mimics the benefits of exercise and help more people to maintain the physical and brain health effects of exercise with a low-cost means (Huang et al., 2021, Front. Physiol., 12:538962). Further, recent findings on lactate-related mechanisms to promote brain health during exercise and cognitive function have provided additional new perspectives for promoting a healthy aging strategy through lactate enhancement (Xue et al., 2022, Nutrition & Metabolism, 19:52).

WO 99/62885 describes the preparation of N-(pyrazolylphenyl)alkanamides as IL-2 production inhibitors.

Given the critical role of ANLS in neuronal protection and cognition and the observed impairment of astrocytes function and brain energy metabolism in a number of neurological diseases including MCI, AD, ALS, GLUT1-DS and depression, there is an emerging need to develop lactate enhancing drugs.

### SUMMARY OF THE INVENTION

The present invention is based on the unexpected findings of new molecules stimulating release of lactate and glucose uptake in primary astrocytes cell cultures in vitro and in mice in vivo and having therapeutic effects in the GLUT1-DS prototypic mouse model of hypometabolism, as well as mouse models of aging and AD.

A first aspect of the invention provides a compound of the invention, as well as pharmaceutically acceptable salts, hydrates, solvates, or polymorphs, tautomers, optically active forms, enantiomeric mixtures thereof and pharmaceutically active derivative and mixtures thereof.

According to another aspect of the invention, is provided a compound of the invention for use as a medicament.

According to another aspect of the invention, is provided a pharmaceutical comprising at least one compound according to the invention and a pharmaceutically acceptable carrier, diluent or excipient thereof as defined herein.

According to another aspect, the invention provides a compound of the invention for use in the prevention and/or treatment of a neurological disorder or any medical condition characterized by an hypometabolism status and/or a dysfunction of the central or peripheral nervous system, which is a disease associated with an abnormally low energy metabolism or in the central or peripheral nervous system or for the treatment or stabilizing a neurological disorder with brain hypometabolism or associated symptoms that include cognitive impairment, motor function and movement disorders or epileptic seizures.

According to another aspect, the invention provides a compound of the invention for use in the prevention and/or treatment of cognitive impairments related to ageing such as, but not limited to, age-associated cognitive decline and age-related memory impairments and for enhancing cognitive and memory functions in healthy subjects.

According to another, the invention provides a use of a compound of the invention as well as pharmaceutically acceptable salts, hydrates, solvates, or polymorphs, tautomers, optically active forms, enantiomeric mixtures thereof and pharmaceutically active derivative and mixtures thereof for the preparation of a pharmaceutical composition for the prevention and/or treatment of a disorder or a disease associated with an abnormally low intracerebral energy metabolism or in the central nervous system and/or a neurological disorder or for the treatment or stabilizing a neurological disorder with brain hypometabolism or associated symptoms.

According to another, the invention provides a method of preventing or treating a disorder or a disease associated with an abnormally low energy metabolism or in the central nervous system and/or a neurological disorder or for the treatment or stabilizing a neurological disorder with brain hypometabolism or associated symptoms in a subject, said method comprising administering in a subject in need thereof a therapeutically effective amount of a compound of the invention, a tautomer, a geometrical isomer, an optically active form, an enantiomeric mixture, a pharmaceutically acceptable salt, a pharmaceutically active derivative thereof or a mixture thereof.

According to another, the invention provides a method of increasing the intracerebral glucose and/or lactate levels in a subject, said method comprising administering in a subject in need thereof an effective amount of a compound of the invention or pharmaceutically acceptable salts, hydrates, solvates, or polymorphs, tautomers, optically active forms, enantiomeric mixtures thereof and pharmaceutically active derivative and mixtures thereof to induce increased intracerebral glucose and/or lactate levels.

A method for enhancing cognitive and memory functions in a subject, said method comprising administering an effective amount of a compound of the invention or pharmaceutically acceptable salts, hydrates, solvates, or polymorphs, tautomers, optically active forms, enantiomeric mixtures thereof and pharmaceutically active derivative and mixtures thereof.

According to another aspect, is provided a process for the preparation of a compound according to Formula (I) comprising the step of reacting an intermediate of Formula (II) with an intermediate of Formula (III) in a polar solvent, wherein the compound of Formula (I) is a compound of Formula (Ia).

According to another aspect, is provided a process for the preparation of a compound according to Formula (I) comprising a step of reducing an intermediate of Formula (IV), wherein the compound of Formula (I) is a compound of Formula (Ib).

### DESCRIPTION OF THE FIGURES

FIG. 1 shows the uptake of glucose from primary cultures of astrocytes measured at 90 min after stimulation with compounds (1) to (5) of the invention at concentrations ranging from 100 nM to 10 μM as described in Example 2, represented as % of treatment with Vehicle+S.E.M, n=6.

FIG. 2 shows in vitro mitochondrial activity of primary astrocytes measured as described in Example 2 at 1.5 and 24 hours after treatment with compounds (1) to (7) of the invention at concentrations ranging from 100 nM to 100 μM, represented as absorbance of MTT colorimetric assay % of treatment with Vehicle+SEM; n=8.

FIG. 3 shows the mitochondrial activity of primary neurons alone or with presence of astrocytes when treated with compound (2) of the invention (10 μM) as described in Example 2, represented as % of treatment with Vehicle±S.E.M, n=6.

FIG. 4 shows glucose uptake and lactate release from human induced-pluripotent stem cells (iPSCs)-derived astrocytes when treated with compound (2) of the invention at concentrations ranging from 10 nM to 10 μM as described in Example 2, represented as % of treatment with Vehicle+S.E.M, n=6.

FIG. 5 shows glucose and lactate extracellular levels in the brain of freely moving mice in vivo using glucose and lactate biosensors, respectively, after oral administration of compounds (1) to (4) of the invention (10 to 30 mg/kg) or vehicle for a duration of 3 hours as described in Example 3, represented as average of AUC ratio of Veh or compounds (1) to (4) of the invention over Veh in the same mouse+S.E.M, n=4-8.

FIG. 6 shows glucose uptake in the brain of mice in vivo using 18F-fluorodeoxyglucose (FDG)-positron emission tomography (PET) after oral administration of compound (1) or (2) of the invention (30 mg/kg each) or vehicle as described in Example 3, represented as average of FDG accumulation (BQML/vol)±S.E.M, n=6.

FIG. 7 shows glucose and lactate extracellular levels in the brain of freely moving GLUT1-DS mice in vivo using glucose and lactate biosensors, respectively, after oral administration of compound (2) of the invention (10 mg/kg) or vehicle for a duration of 3 hours as described in Example 4, represented as average of AUC ratio of Veh or compound (2) of the invention over Veh in the same mouse+S.E.M, n=4-8.

FIG. 8 represents the latency of Wild-type (WT) or GLUT1-DS mice before falling down the rotarod at a speed accelerating from 4 r.p.m. to 40 r.p.m. over 300 seconds 20 min after oral administration of compound (1) or (2) of the invention (10 mg/kg) or vehicle, as described in Example 4 (A-B). Data are shown as the average+S.E.M of n=16 (n=8 males, n=8 females). (C-D) Data represents the grip strength (Newton) of the 4 paws of WT and GLUT1-DS mice using grip strength test after 20 min after oral administration of compound (1) or (2) of the invention (10 mg/kg) or vehicle, as described in Example 4. Data are shown as average+S.E.M of n=16.

FIG. 9 represents the distance of young (3-month-old) and old (16-month-old) wild-type mice to reach the target location in the Morris Water Maze at 1 day after training, following oral administration of vehicle or compound (2) of the invention (10 mg/kg and 30 mg/kg), as described in Example 5. Data are shown as the average+S.E.M. of the distance (cm) of an n=5.

FIG. 10 represents the distance of 3-month-old APOE3(+) and APOE4(+) female and male mice to reach the target location in the Morris Water Maze at 7 days after training, following oral administration of vehicle or compound (2) of the invention (10 mg/kg and 30 mg/kg), as described in Example 5. Data are shown as the average+S.E.M. of the distance (cm) of an n=20 (n=10 males, n=10 females).

FIG. 11 represents the memory of saline (CTL) or streptozotocin (STZ) intracerebroventricular injected mice treated orally with vehicle or compound (2) of the invention (10 mg/kg and 30 mg/kg), as assessed by the distance to reach the target location in the Morris Water Maze at 1 day after training (A), by the preference index of novel vs. known object in the Novel Object Recognition task at 1 day after training (B), and by the latency to enter the dark compartment in the Inhibitory Avoidance task at 1 day after training (C), as described in Example 5. Data are shown as the average+S.E.M. of the distance (cm) (A), of the preference index (%) (B), or of the latency (sec) (C) of an n=13-16 (n=5-8 males, n=8 females).

### DETAILED DESCRIPTION

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it such as a preventive early asymptomatic intervention; (b) inhibiting the disease, i.e., arresting its development; or relieving the disease, i.e., causing regression of the disease and/or its symptoms or conditions such as improvement or remediation of damage. In particular, the methods, uses, formulations and compositions according to the invention are useful for enhancing lactate, in particular in the treatment of abnormal energy metabolism in the central nervous system.

The term “subject” as used herein refers to mammals. For example, mammals contemplated by the present invention include human, primates, domesticated animals such as cattle, sheep, pigs, horses, laboratory rodents, other pets and the like.

The term “subject at risk of suffering from a disorder related to a deficiency in intracerebral energy metabolism or in the central nervous system (CNS)” refers to a subject presenting abnormal CNS energy metabolism, such as in neurological diseases.

The term “neurological disorder” according to the invention includes a neurological disease or any medical condition characterized by an hypometabolic status and/or a dysfunction of the central or peripheral nervous system, as found in motor neuron diseases (MNDs), such as amyotrophic lateral sclerosis (ALS); in dementias such as Alzheimer's disease, frontotemporal dementia (FTD), dementia with Lewy bodies (LBD), mild cognitive impairments (MCI), vascular dementia, Progressive Supranuclear Palsy (PSP), Multiple System Atrophy (MSA); in movement disorders such as Parkinson's disease including L-Dopa induced dyskinesia, Huntington disease, Spino-cerebellar Ataxias, Essential Tremor, Dystonias and related neurodegenerative conditions; in all aspects of multiple sclerosis; in all types of retinopathy; in stroke, traumatic brain injury, intracerebral- and subarachnoid-haemorrhage; in a neuropsychiatric condition such as any endophenotype of depression, schizophrenia, anxiety, attention-deficit syndrome, autism; in a neurometabolic disorder such as glucose transporter 1 deficiency syndrome (GLUT1-DS), Lafora disease and other glycogen storage disorders; in Down Syndrome; in all types of epilepsy, migraine and cognitive impairments in Type 2 diabetes (T2D); in brain hypometabolism status caused by viral infection such as HIV or COVID-19, by prion infection such as in Creutzfeldt-Jakob disease; in primary and secondary encephalitis; in any brain hypometabolism status following anaesthesia or post-operative care. The term “GLUT1-DS” according to the invention includes GLUT1 deficiency syndrome, Glucose transporter 1 deficiency syndrome, GLUT1 deficiency disorder, also known as De Vivo syndrome, De Vivo disease or De Vivo syndrome disease.

The term “effective amount” as used herein refers to an amount of at least one compound of the invention or a pharmaceutical formulation thereof according to the invention that elicits the biological or medicinal response in a tissue, system, animal or human that is being sought. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of compound of the invention sufficient to reduce the progression of the disease, notably to reduce or inhibit the progression of a neurodegenerative disorder and thereby elicit the response being sought (i.e. an “effective amount”).

The term “efficacy” of a treatment according to the invention can be measured based on changes in the course of disease in response to a use or a method according to the invention. For example, the efficacy of a treatment can be measured by an increase of glucose or lactate levels in the central nervous system, as well as by imaging techniques including Positron Emission Tomography (PET) with fluorine-18 (¹⁸F)-labeled 2-fluoro-2-deoxy-D-glucose as tracer or carbon-11, (¹¹C) Pittsburgh compound B (PIB), carbon-13 (¹³C), phosphorus-31 (³¹P), proton magnetic resonance spectroscopy (¹H) MRS to evaluate the bioenergetics status in the brain. Effective treatment is indicated by an increase in cognitive performance (e.g. memory, reasoning test), preservation of neuronal activity, which, in the case of motor dysfunction, can be measured by muscle activity, as well as clinical diagnosis relevant to a specific indication. The term “C₁-C₆ alkyl” when used alone or in combination with other terms, comprises a straight chain or branched C₁-C₆ alkyl which refers to monovalent alkyl groups having 1 to 6 carbon atoms. This term is exemplified by groups such as methyl, ethyl, n-propyl, i-propyl, n-butyl, s-butyl, i-butyl, t-butyl, n-pentyl, 1-ethylpropyl, 2-methylbutyl, 3-methylbutyl, 2,2-dimethylpropyl, n-hexyl, 2-methylpentyl, 3-methylpentyl, 4-methylpentyl and the like.

The term “C₂-C₆ alkenyl” when used alone or in combination with other terms, comprises a straight chain or branched C₂-C₆ alkenyl. It may have any available number of double bonds in any available positions, and the configuration of the double bond may be the (E) or (Z) configuration. This term is exemplified by groups such as vinyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl, 5-hexenyl, and the like.

The term “C₃-C₈ heterocycle” includes “C₃-C₈ heterocycloalkyl” and “heteroaryl”.

The term “C₃-C₈ heterocycloalkyl” refers to a C₃-C₈-cycloalkyl in which up to 3 carbon atoms are replaced by heteroatoms chosen from the group consisting of O, S, NR, R being defined as hydrogen or methyl. Heterocycloalkyl include pyrrolidinyl, piperidinyl, piperazinyl, morpholinyl, tetrahydrofuranyl and the like.

The term “heteroaryl” refers to a monocyclic heteroaromatic, or a bicyclic or a tricyclic fused-ring heteroaromatic group. Particular examples of heteroaromatic groups include optionally substituted pyridyl, pyrrolyl, pyrimidinyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl,1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

Unless otherwise constrained by the definition of the individual substituent, the term “substituted” refers to groups substituted with from 1 to 5 substituents selected from the group consisting of “C₁-C₆ alkyl,” “C₃-C₅-cycloalkyl,” “heterocycloalkyl,” “C₁-C₆ alkyl aryl,” “C₁-C₆ alkyl heteroaryl,” “C₁-C₆ alkyl cycloalkyl,” “C₁-C₆ alkyl heterocycloalkyl,” “cycloalkyl C₁-C₆ alkyl,” “heterocycloalkyl C₁-C₆ alkyl,” “amino,” “aminosulfonyl,” “ammonium,” “alkoxy,” “acyl amino,” “amino carbonyl,” “aryl,” “aryl C₁-C₆ alkyl,” “heteroaryl,” “heteroaryl C₁-C₆ alkyl,” “sulfinyl,” “sulfonyl,” “sulphonamide”, “alkoxy,” “alkoxy carbonyl,” “carbamate,” “sulfanyl,” “halogen,” “carboxy,” trihalomethyl, cyano, hydroxy, mercapto, nitro, trihalo methyloxy, trihalo methylthio and the like.

“Pharmaceutically active derivative” refers to any compound that upon administration to the recipient, is capable of providing directly or indirectly, the activity disclosed herein. The term “indirectly” also encompasses prodrugs which may be converted to the active form of the drug via endogenous enzymes or metabolism. The prodrug is a derivative of the compound according to the invention and presenting lactate enhancing activity that has a chemically or metabolically decomposable group, and a compound that may be converted into a pharmaceutically active compound in vivo under physiological conditions.

The prodrug is converted into a compound according to the present invention by a reaction with an enzyme, gastric acid or the like under a physiological condition in the living body, e.g. by oxidation, reduction, hydrolysis or the like, each of which is carried out enzymatically.

These compounds can be produced from compounds of the present invention according to well-known methods. The term “indirectly” also encompasses metabolites of compounds according to the invention.

The term “metabolite” refers to all molecules derived from any of the compounds according to the present invention in a cell or organism, preferably mammal.

In the context of the present invention are encompassed pharmaceutically acceptable salts, hydrates, solvates, or polymorphs, tautomers, optically active forms, enantiomeric mixtures thereof, and mixtures thereof, and pharmaceutically active derivatives of compounds of the invention. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers. Many organic compounds exist in optically active forms having the ability to rotate the plane of plane-polarized light. In describing an optically active compound, the prefixes D and L or R and S are used to denote the absolute configuration of the molecule about its chiral center(s).

In the context of the present invention, «pharmaceutically acceptable salt thereof» refers to salts which are formed from acid addition salts formed with an acid, said acid may be an inorganic acid (e.g. hydrochloric acid, hydrobromic acid, sulfuric acid, phosphoric acid, nitric acid, and the like), or an organic acid such as acetic acid, fumaric acid, oxalic acid, tartaric acid, succinic acid, malic acid, malonic acid, fumaric acid, maleic acid, ascorbic acid, lactic acid or benzoic acid.

Pharmaceutically acceptable salts for the instant disclosure may be selected among salts formed with acids such as hydrochloric acid.

The term “pharmaceutical formulation” refers to preparations which are in such a form as to permit biological activity of the active ingredient(s) to be unequivocally effective and which contain no additional component which would be toxic to subjects to which the said formulation would be administered.

### Compounds for Use According to the Invention

According to a particular aspect of the invention, are provided compounds of Formula (I):

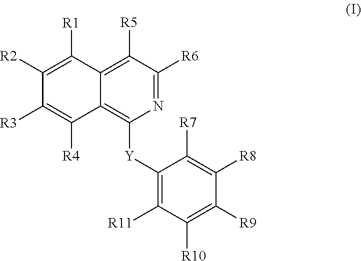

Wherein Y is selected from NH and CH₂; R1 is selected from H, halogen and C₁-C₆ alkyl, optionally substituted with a group selected from halogen, OR¹² and NR¹³R¹⁴; R2 is selected from H, halogen, C₁-C₆ alkyl, optionally substituted with a group selected from halogen, OR¹² and NHR¹³, or C₂-C₆ alkenyl, or a group OR¹², NR¹³R¹⁴⁻ or a cyano group or an optionally substituted heterocycle (e.g. optionally substituted 6-pyrimidin, optionally substituted azetidine); R3 is selected from H, halogen (e.g. Br), C₁-C₆ alkyl, optionally substituted with a group selected from halogen, OR¹² and NR¹³R¹⁴⁻ or C₂-C₆ alkenyl, or a group OR¹², or NHR¹³ or an optionally substituted heterocycle or a cyano group; R4 is selected from H, halogen C₁-C₆ alkyl, optionally substituted with a group selected from halogen, OR¹² and NHR¹³, or an optionally substituted heterocycle and a cyano group; R5 is selected from H, halogen, C₁-C₆ alkyl, optionally substituted with a group selected from halogen, OR¹² and NHR¹³, or a group OR¹², or NR¹³R4; R6 is selected from H, halogen, C₁-C₆ alkyl, optionally substituted with a group selected from halogen, OR¹² and NHR¹³, or a group OR¹², or NHR¹³; R7 and R8 are independently selected from H and halogen; R9 is selected from SO—C₁-C₆ alkyl, SO₂—C₁-C₆ alkyl, SO₂—C₃-C₆ cycloalkyl, or an optionally substituted heterocycle selected from optionally substituted imidazole, optionally substituted isoxazole, optionally substituted oxazole, optionally substituted pyridine, optionally substituted pyrimidine, optionally substituted pyrrolinone (e.g. pyrrolidin-2-one), and optionally substituted oxetane; R10 and R11 are independently selected from H and halogen; R12, R13, and R14 are independently selected from H, C(O)— C₁-C₆ alkyl (e.g. CO-methyl) and optionally substituted C₁-C₆ alkyl or C₃-C₆ cycloalkyl (e.g. optionally substituted ethyl, such as fluoro ethyl, ethyl phenyl ethyl, cyclopropyl methyl, optionally substituted propyl); any pharmaceutically acceptable salts, hydrates, solvates, or polymorphs, tautomers, optically active forms, enantiomeric mixtures thereof, and mixtures thereof, for the prevention, the repression or treatment of neurological disease or any medical condition characterized by an hypometabolic status and/or a dysfunction of the central or peripheral nervous system, in particular in motor neuron diseases (MNDs) such as amyotrophic lateral sclerosis (ALS), dementia, in particular Alzheimer's disease, frontotemporal dementia (FTD), dementia with Lewy bodies (LBD), mild cognitive impairments (MCI), vascular dementia, Progressive Supranuclear Palsy (PSP), Multiple System Atrophy (MSA), movement disorders such as Parkinson's disease including L-Dopa Induced dyskinesia, Huntington disease, Spino-cerebellar ataxias, Essential Tremor, Dystonias and related neurodegenerative conditions, multiple sclerosis, retinopathies, stroke, traumatic brain injury, intracerebral- and subarachnoid-haemorrhage, a neuropsychiatric disorder such as any endophenotype of depression, schizophrenia, anxiety, attention-deficit syndrome, autism, a neurometabolic disorder such as glucose transporter 1 deficiency syndrome (GLUT1-DS), Lafora disease and other glycogen storage disorders, Down Syndrome, all types of epilepsy, migraine and cognitive impairments in Type 2 diabetes (T2D), brain hypometabolism status caused by viral infection such as HIV or COVID-19, by prion infection such as in Creutzfeldt-Jakob disease, primary and secondary encephalitis, or by abnormal protein processing and accumulation such as all types of amyloidopathies, synucleinopathies, tauopathies, TD43opathies and other proteinopathies, brain hypometabolism status following anaesthesia or post-operative care or for the treatment or stabilizing a neurological disorder with brain hypometabolism or associated symptoms that include cognitive impairment, motor function, psychiatric symptoms and movement disorders or epileptic seizures and for enhancing cognitive and memory functions.

According to a particular embodiment, is provided a compound of Formula (I) wherein Y is CH₂.

According to a particular embodiment, is provided a compound of Formula (I) wherein Y is NH.

According to a particular embodiment, is provided a compound of Formula (I) wherein R1, R5, R4 and R6 are H.

According to a particular embodiment, is provided a compound of Formula (I) wherein R2 is OR¹².

According to a particular embodiment, is provided a compound of Formula (I) wherein R3 is OR¹².

According to a particular embodiment, is provided a compound of Formula (I) wherein R3 is NHR¹³.

According to a particular embodiment, is provided a compound of Formula (I) wherein R3 is halogen.

According to a particular embodiment, is provided a compound of Formula (I) wherein R3 is H.

According to a particular embodiment, is provided a compound of Formula (I) wherein R3 optionally substituted C₁-C₆ alkyl (e.g. optionally substituted propyl).

According to a particular embodiment, is provided a compound of Formula (I) wherein R12 is optionally substituted C₁-C₆ alkyl (e.g. optionally substituted methyl, ethyl (e.g ethyl or fluoro ethyl), isopropyl, optionally substituted aryl C₁-C₆ alkyl such as optionally substituted phenyl C₁-C₆ alkyl like fluorophenyl methyl).

According to a particular embodiment, is provided a compound of Formula (I) wherein R12 is H.

According to a particular embodiment, is provided a compound of Formula (I) wherein R13 is optionally substituted C₁-C₆ alkyl (e.g. optionally substituted ethyl, such as fluoroethyl, ethyl phenyl ethyl, cyclopropyl methyl, optionally substituted propyl).

According to a particular embodiment, is provided a compound of Formula (I) wherein R10 and R11 are H.

According to a particular embodiment, is provided a compound of Formula (I) wherein R10 is halogen for example fluoro.

According to a particular embodiment, is provided a compound of Formula (I) wherein R11 is halogen for example fluoro.

According to a particular embodiment, is provided a compound of Formula (I) wherein R7, R8, R10 and R11 are H.

According to a particular embodiment, is provided a compound of Formula (I) wherein R9 is selected from SO₂—C₁-C₆ alkyl such as SO₂—CH₃ or SO₂—CH₂CH₃.

According to a particular embodiment, is provided a compound of Formula (I) wherein R9 is selected from the following groups:

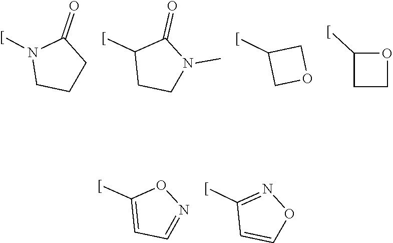

According to a particular embodiment, is provided a compound of Formula (I) wherein R9 is optionally substituted pyrrolidin-2-one.

According to a particular embodiment, is provided a compound of Formula (I) wherein R9 is optionally substituted oxetane.

According to a particular embodiment, is provided a compound of Formula (I) wherein R9 is optionally substituted isoxazole.

According to a particular embodiment, is provided a compound of Formula (I) wherein R9 is optionally substituted SO₂-cyclopropyl.

In a more particular embodiment, compounds for use according to the invention are selected from the following group:
1-[4-[(6,7-dimethoxy-1-isoquinolyl)methyl]phenyl]pyrrolidin-2-one;
6,7-dimethoxy-N-(4-methylsulfonylphenyl)isoquinolin-1-amine;
6-methoxy-1-(4-methylsulfonylanilino)isoquinolin-7-ol;
7-methoxy-1-(4-methylsulfonylanilino)isoquinolin-6-ol;
1-(4-methylsulfonylanilino)isoquinoline-6,7-diol;
6-ethoxy-1-(4-methylsulfonylanilino)isoquinolin-7-ol;
7-ethoxy-1-(4-methylsulfonylanilino)isoquinolin-6-ol;
6,7-diethoxy-N-(4-methylsulfonylphenyl)isoquinolin-1-amine;
1-[4-[(6,7-dimethoxy-1-isoquinolyl)amino]phenyl]pyrrolidin-2-one;
N-(4-methylsulfonylphenyl)-6-vinyloxy-isoquinolin-1-amine;
6-isopropoxy-N-(4-methylsulfonylphenyl)isoquinolin-1-amine;
N-(4-methylsulfonylphenyl)-6-pyrimidin-2-yl-isoquinolin-1-amine;
N-[1-(4-methylsulfonylanilino)-7-isoquinolyl]acetamide;
N7-ethyl-N1-(4-methylsulfonylphenyl)isoquinoline-1,7-diamine;
6-methoxy-N-(4-methylsulfonylphenyl)-7-vinyl-isoquinolin-1-amine;
7-ethyl-6-methoxy-N-(4-methylsulfonylphenyl)isoquinolin-1-amine;
7-bromo-1-(4-methylsulfonylanilino)isoquinolin-6-ol;
N7-benzyl-6-methoxy-N1-(4-methylsulfonylphenyl)isoquinoline-1,7-diamine;
N7-(cyclopropylmethyl)-6-methoxy-N1-(4-methylsulfonylphenyl)isoquinoline-1,7-diamine;
6-methoxy-N1-(4-methylsulfonylphenyl)-N7-propyl-isoquinoline-1,7-diamine;
N6-(cyclopropylmethyl)-7-methoxy-N1-(4-methylsulfonylphenyl)isoquinoline-1,6-diamine;
6-(azetidin-1-yl)-7-methoxy-N-(4-methylsulfonylphenyl)isoquinolin-1-amine;
6-ethyl-1-[(4-methanesulfonylphenyl)amino]isoquinolin-7-ol;
N-[4-(ethanesulfonyl)phenyl]-6,7-diethoxyisoquinolin-1-amine;
6,7-diethoxy-N-[4-(oxetan-3-yl)phenyl]isoquinolin-1-amine;
6,7-diethoxy-N-[4-(1,2-oxazol-3-yl)phenyl]isoquinolin-1-amine;
6,7-diethoxy-N-[4-(1,2-oxazol-5-yl)phenyl]isoquinolin-1-amine;
6,7-diethoxy-N-(3-fluoro-4-methanesulfonylphenyl)isoquinolin-1-amine;
6,7-diethoxy-N-(2-fluoro-4-methanesulfonylphenyl)isoquinolin-1-amine;
N-[4-(cyclopropanesulfonyl)phenyl]-6,7-diethoxyisoquinolin-1-amine;
6-ethoxy-7-(2-fluoroethoxy)-N-(4-methanesulfonylphenyl)isoquinolin-1-amine;
6,7-bis(2-fluoroethoxy)-N-(4-methanesulfonylphenyl)isoquinolin-1-amine;
1-[(4-methanesulfonylphenyl)methyl]-6-(propan-2-yloxy)isoquinoline;
6-[(4-fluorophenyl)methoxy]-N-(4-methanesulfonylphenyl)isoquinolin-1-amine; and
6-ethoxy-N-(4-methanesulfonylphenyl)-7-propylisoquinolin-1-amine;
any pharmaceutically acceptable salts, hydrates, solvates, or polymorphs, tautomers, optically active forms, enantiomeric mixtures thereof, and mixtures thereof.

According to a further particular aspect of the invention, are provided compounds of Formula (I); any pharmaceutically acceptable salts, hydrates, solvates, or polymorphs, tautomers, optically active forms, enantiomeric mixtures thereof, and mixtures thereof, with the proviso that said compound is not a compound selected from the following list:
N-[4-[5-Ethyl-3-(1-methylethyl)-1H-pyrazol-1-yl]phenyl]-1-isoquinolinamine, RN: 1101888-63-4;
N-[4-[5-Chloro-3-(trifluoromethyl)-1H-pyrazol-1-yl]phenyl]-1-isoquinolinamine, RN: 251657-99-5;
N-[4-[5-Ethyl-3-(3-pyridinyl)-1H-pyrazol-1-yl]phenyl]-1-isoquinolinamine, RN: 251658-04-5;
N-[4-[3,5-Bis(trifluoromethyl)-1H-pyrazol-1-yl]phenyl]-1-isoquinolinamine, RN: 251657-94-0;
N-[4-[3-(Tetrahydro-2-furanyl)-5-(trifluoromethyl)-1H-pyrazol-1-yl]phenyl]-1-isoquinolin amine, RN: 1101888-82-7;
N-[4-[3-(3-Pyridinyl)-5-(trifluoromethyl)-1H-pyrazol-1-yl]phenyl]-1-isoquinolinamine, RN: 251658-03-4;
3-Methyl-N-[4-(4-pyridinyl)phenyl]-1-isoquinolinamine, RN: 1368370-93-7;
1-[[4-(4-Pyridinyl)phenyl]amino]-8-isoquinolinecarbonitrile, RN:1368269-53-7;
8-Methyl-N¹-[4-(4-pyridinyl)phenyl]-1,5-isoquinolinediamine, RN:1369288-67-4;
5-Nitro-N-[4-(4-pyridinyl)phenyl]-1-isoquinolinamine, RN:1368370-43-7;
N-[4-(4-Pyridinyl)phenyl]-5-(trifluoromethyl)-1-isoquinolinamine, RN: 1367803-95-9; and
4-Bromo-N¹-[4-(4-pyridinyl)phenyl]-1,7-isoquinolinediamine, 1369271-85-1.

The compounds of invention have been named according to the IUPAC standards used in ChemAxon Marvin Sketch version 20.11.0.

According to another aspect the invention, a process for the preparation of a compound according to Formula (I) comprises the step of reacting an aniline intermediate of Formula (III) with an intermediate of Formula (II) wherein Z is a leaving group selected from Iodide, Bromide, Chloride, O-triflate or the like in a polar solvent to form a compound of Formula (Ia) (Scheme 1):

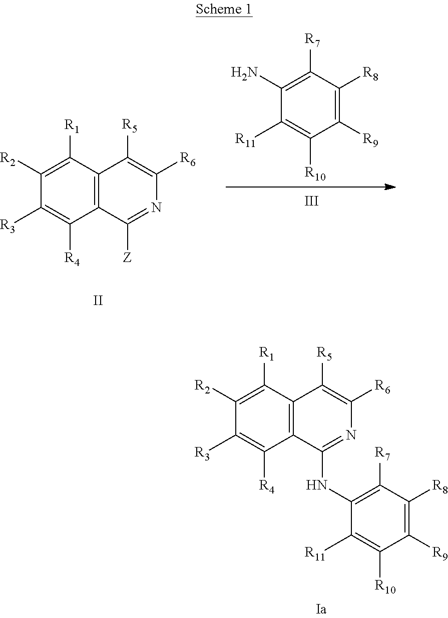

According to another aspect, a process for the preparation of a compound according to Formula (I) comprises a reduction step of a carbonyl intermediate of Formula (IV) (e.g. by NaBH₃CN in the presence of ZnCl₂ or by catalytic hydrogenation (H₂ in the presence of Pd/C and acid trace) to lead to a compound of Formula (Ib) (Scheme 2):

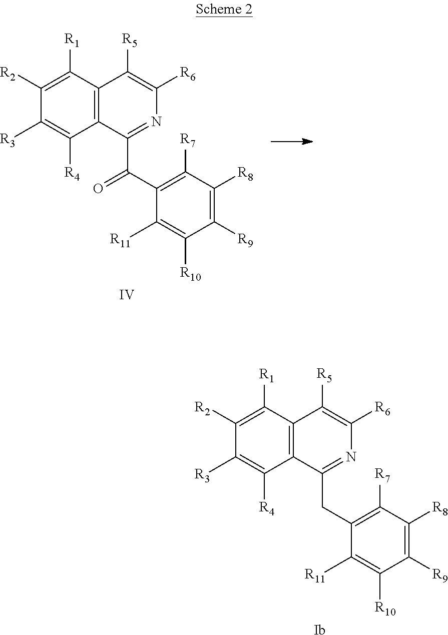

### Compositions According to the Invention

The invention provides pharmaceutical or therapeutic agents as compositions and methods useful for treating a subject, preferably a mammalian subject, and most preferably a human patient who is suffering from a medical disorder, and in particular a disease or disorder as defined therein.

According to a further particular aspect of the invention, is provided a pharmaceutical comprising at least one compound according to Formula (I), with the proviso that the compounds is not a compound selected from the following list:
N-[4-[5-Ethyl-3-(1-methylethyl)-1H-pyrazol-1-yl]phenyl]-1-isoquinolinamine, RN: 1101888-63-4;
N-[4-[5-Chloro-3-(trifluoromethyl)-1H-pyrazol-1-yl]phenyl]-1-isoquinolinamine, RN: 251657-99-5;
N-[4-[5-Ethyl-3-(3-pyridinyl)-1H-pyrazol-1-yl]phenyl]-1-isoquinolinamine, RN: 251658-04-5;
N-[4-[3,5-Bis(trifluoromethyl)-1H-pyrazol-1-yl]phenyl]-1-isoquinolinamine, RN: 251657-94-0;
N-[4-[3-(Tetrahydro-2-furanyl)-5-(trifluoromethyl)-1H-pyrazol-1-yl]phenyl]-1-isoquinolin amine, RN: 1101888-82-7; and
N-[4-[3-(3-Pyridinyl)-5-(trifluoromethyl)-1H-pyrazol-1-yl]phenyl]-1-isoquinolinamine, RN: 251658-03-4.

The agent of the invention or formulations thereof may be administered as a pharmaceutical formulation, which can contain one or more agents according to the invention in any form described herein. The compositions according to the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous) use by injection or continuous infusion. Injectable compositions are typically based upon injectable sterile saline or phosphate-buffered saline or other injectable carriers known in the art. Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Compositions of this invention may be liquid formulations including, but not limited to aqueous or oily suspensions, solutions, emulsions, syrups, and elixirs. The compositions may also be formulated as a dry product for reconstitution with water or other suitable vehicle before use. Such liquid preparations may contain additives including, but not limited to, suspending agents, emulsifying agents, non-aqueous vehicles and preservatives. Suspending agents include, but are not limited to, sorbitol syrup, methylcellulose, glucose/sugar syrup, gelatin, hydroxyethyl cellulose, carboxymethyl cellulose, aluminum stearate gel, and hydrogenated edible fats. Emulsifying agents include, but are not limited to, lecithin, sorbitan monooleate, and acacia. Preservatives include, but are not limited to, methyl or propyl p-hydroxybenzoate and sorbic acid. Dispersing or wetting agents include but are not limited to poly(ethylene glycol), glycerol, bovine serum albumin, Tween®, Span®.

Compositions of this invention may also be formulated as a depot preparation, which may be administered by implantation or by intramuscular injection.

Solid compositions of this invention may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maize starch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate. Tablets may be coated according to methods well known in the art.

The compounds of this invention can also be administered in sustained release forms or from sustained release drug delivery systems.

According to a particular embodiment, compositions according to the invention are for intravenous use.

According to a particular aspect, the formulations of the invention are oral formulations.

In another particular aspect, the compositions according to the invention are adapted for delivery by repeated administration.

According to a particular embodiment, compositions of the invention are veterinary compositions.

Further materials as well as formulation processing techniques and the like are set out in Remington: The Science & Practice of Pharmacy, 23^{rd} Edition, 2020, Ed. Adeboye Adejare, which is incorporated herein by reference.

### Mode of Administration

Compounds and formulations thereof according to this invention may be administered in any manner including orally, nasally, parenterally, intravenously, intrathecally, rectally, ophthalmologically and the like or combinations thereof. Compounds and formulations thereof according to this invention may be also administered by inhalation or intradermally. Parenteral administration includes, but is not limited to, intravenous, intra-arterial, intra-peritoneal, subcutaneous and intramuscular. The compositions of this invention may also be administered in the form of an implant, which allows slow release of the compositions as well as a slow controlled i.v. infusion.

According to a particular embodiment, the compounds and formulations thereof according to this invention are to be administered by oral route.

### Combination

According to the invention, compounds and pharmaceutical formulations thereof can be administered alone or in combination with a co-agent useful for treating, and/or stabilizing a neurological disorder or any medical condition characterized by an hypometabolic status and/or a dysfunction of the central or peripheral nervous system, such as a motor neuron diseases (MNDs), in particular amyotrophic lateral sclerosis (ALS), dementia, in particular Alzheimer's disease, frontotemporal dementia (FTD), dementia with Lewy bodies (LBD), mild cognitive impairments (MCI), vascular dementia, Progressive Supranuclear Palsy (PSP), Multiple System Atrophy (MSA), movement disorders such as Parkinson's disease, Huntington disease, Spino-cerebellar ataxias, Essential Tremor, Dystonias and related neurodegenerative conditions, multiple sclerosis, retinopathies, stroke, traumatic brain injury, intracerebral- and subarachnoid-haemorrhage, a neuropsychiatric disorder such as any endophenotype of depression, schizophrenia, anxiety, attention-deficit syndrome, autism, a neurometabolic disorder such as GLUT1-DS, Lafora disease and other glycogen storage disorders, Down Syndrome, all types of epilepsy, migraine and cognitive impairments in Type 2 diabetes (T2D), brain hypometabolism status caused by viral infection such as HIV or COVID-19, by prion infection such as in Creutzfeldt-Jakob disease, primary and secondary encephalitis, brain hypometabolism status following anaesthesia or post-operative care.

According to the invention, compounds and pharmaceutical formulations thereof can be administered alone or in combination with a co-agent or a co-treatment useful for treating, and/or stabilizing used for the treatment of a neurological disorder with brain hypometabolism or associated symptoms that include cognitive impairment, motor function and movement disorders or epileptic seizures. Such co-agents or co-treatments would include, but would not be limited to, at least co-agent useful for treating and/or stabilizing a neurological disorder with brain hypometabolism or associated symptoms including gene therapy for restoring expression of gene(s) involved in brain energy metabolism, a ketogenic diet or a pharmaceutical compound regulating the synthesis of ketone bodies such as, for example, triheptanoin.

The invention encompasses the administration of a compound of the invention or a formulation thereof wherein it is administered to a subject prior to, simultaneously or sequentially with other therapeutic regimens or co-agents useful for the prevention and/or treatment of psychiatric disorders or improving cognitive and memory functions.

Examples of co-agents useful in combination with compounds of the invention and pharmaceutical formulations thereof include drug therapies useful for the treatment of the cognitive symptoms (memory loss, confusion, and problems with thinking and reasoning) of Alzheimer's disease such as cholinesterase inhibitors and memantine and amyloid-targeting agents. Non-limiting examples of cholinesterase inhibitors include donepezil, rivastigmine and galantamine. Non-limiting amyloid-targeting agents include Aducanumab.

A co-agent according to the invention may include donepezil and memantine in a single dosage form.

Examples of co-agents useful in combination with compounds of the invention and pharmaceutical formulations thereof include drug therapies useful to treat Amyotrophic Lateral Sclerosis such as riluzole, edaravone, AMX0035 (sodium phenyltbutyrate and ursodoxicoltaurine) and Nuedexta (dextromethorphan and quinidine).

Examples of co-agents useful in combination with compounds of the invention include medications for behavioural changes, which act as adjunct treatments but which do not directly treat the symptoms of Alzheimer's disease, such as one or more of antidepressants, anxiolytics or antipsychotic medications. Non-limiting examples of suitable antidepressants include citalopram, fluoxetine, paroxetine, sertraline, trazodone and esketamine. Non-limiting examples of suitable anxiolytics include lorazepam and oxazepam. Non-limiting examples of suitable antipsychotic medications include aripiprazole, clozapine, haloperidol, olanzapine, quetiapine, risperidone and ziprasidone.

A compound of the invention or a formulation thereof according to the invention that is administered simultaneously with said co-agents can be administered in the same or different composition(s) and by the same or different route(s) of administration.

According to one embodiment, is provided a pharmaceutical formulation comprising a compound of the invention combined with at least one co-agent useful for treating, and/or stabilizing, a neurodegenerative disorder and at least one pharmaceutically acceptable carrier.

Other combinations will be readily appreciated and understood by persons skilled in the art. In some embodiments, the compounds of the invention can be used to attenuate or reverse the activity of a drug suitable for treatment of a neurological disease as described herein, and/or limit the adverse effects of such drugs.

As persons skilled in the art will readily understand, the combination can include the therapeutic agents and/or a pharmaceutical composition comprising same, according to at least some embodiments of the invention and one other drug; the therapeutic agents and/or a pharmaceutical composition comprising same, as recited herein, with two other drugs, the therapeutic agents and/or a pharmaceutical composition comprising same, as recited herein, with three other drugs, etc. The determination of the optimal combination and dosages can be determined and optimized using methods well known in the art.

The therapeutic agent according to the present invention and one or more other therapeutic agents can be administered in either order or simultaneously.

### Uses of Compounds According to the Invention

According to another aspect, the invention provides compounds and methods useful for preventing or treating a disorder related to a deficiency in energy metabolism in the central nervous system.

According to another aspect, the invention provides compounds and methods useful for preventing and/or treating a neurodegenerative disorder.

According to another aspect, the invention provides compounds and methods useful for preventing and/or treating a neuropsychiatric disorder.

According to another aspect, the invention provides compounds and methods useful for increasing the lactate secretion by astrocytes.

The dosage administered, as single or multiple doses, to an individual will vary depending upon a variety of factors, including pharmacokinetic properties, patient conditions and characteristics (sex, age, body weight, health, size), extent of symptoms, concurrent treatments, frequency of treatment and the effect desired.

In another embodiment, the invention provides a pharmaceutical composition containing at least one compound of the invention and a pharmaceutically acceptable carrier, diluent or excipient thereof.

### Synthesis of Compounds of the Invention

The novel derivatives according to Formula (I) can be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures. The general synthetic approaches for obtaining compounds of Formula (I) are depicted in Schemes 1 and 2 above.

### Patients

In an embodiment, patients according to the invention are subjects suffering from a disorder related to a deficiency in energy metabolism in the central nervous system.

In a particular embodiment, patients according to the invention are suffering from a neurodegenerative disorder.

In a particular embodiment, patients according to the invention are suffering from a neuropsychiatric disorder.

In a particular embodiment, patients according to the invention are suffering from a neurometabolic disorder.

In an embodiment, patients according to the invention are subjects suffering from a neurometabolic disorder related to GLUT1-DS.

In a particular embodiment, patients according to the invention are suffering from GLUT1-DS.

In an embodiment, patients according to the invention are patients that are at risk of suffering from GLUT1-DS.

In a particular embodiment, patients according to the invention are suffering subjects genetically pre-disposed in suffering from a disorder selected from mild cognitive impairments, Parkinson's disease, multiple sclerosis, schizophrenia, stroke, traumatic brain injury and epilepsy.

According to a particular aspect, compounds and methods of the invention are useful for the prevention and/or treatment of a neurodegenerative disorder.

According to a further aspect, a neurodegenerative disorder is Alzheimer's disease.

According to a further aspect, a neurodegenerative disorder is amyotrophic lateral sclerosis (ALS).

According to another further aspect, a neuropsychiatric disorder is depression.

According to another particular aspect, patients are suffering from mild cognitive impairments due to ageing such as age-associated cognitive decline and age-related memory impairments.

According to another particular aspect, methods of the invention are useful for enhancing cognitive and memory functions in healthy subjects.

The invention having been described, the following examples are presented by way of illustration, and not limitation.

### EXAMPLES

The following studies are conducted to support the effectiveness of compounds of the invention according to the invention.

### Example 1: Synthesis of Compounds of the Invention

All synthetic reagents and solvents are used as is. If necessary, the solvents used in the reactions are previously dried and/or distilled in accordance with the state of the art. Some solvents are commercially available in an anhydrous state and are used as is.

### Reactive Conditions

When anhydrous conditions are required, the glassware is first dried in an oven (>100° C.). All reactions were performed under a nitrogen or argon atmosphere. The ambient temperature (rt) refers to 20 to 25° C. A temperature of −78° C. is obtained by freezing a bath of acetone with carboglace or liquid nitrogen. The temperature of 0° C. corresponds to the use of a water/ice bath. For heating, an oil bath with a temperature sensor is used for temperature control.

The progress of the reaction is followed by thin layer chromatography (CCM) with the aid of UV indicators on the plates and can be enhanced by oxidative developers such as phosphomolybdic acid solutions.

### Purification Techniques

### Flash Chromatography:

Silica gel (Kieselgel 60 from MN, 15-40 μm from Macherey-Nagel) is used in the purification of raw products by Flash chromatography. The samples are either deposited directly at the column head or applied as a solution in a silica gel suspension.

### Automated Chromatography Flash:

The purification system used is a Combiflash Companion™ from Teledyne Isco. The raw samples are dissolved in a small amount of suitable solvent and applied to pre-conditioned RediSep® columns. These columns are placed in the Combiflash Companion purification system™ and purification is performed using a solvent gradient program. The system is used with an automated collector. The detection is carried out by UV or by the collection of all the fractions analysed by HPLC.

### Nuclear Magnetic Resonance (NMR) Spectrometry:

NMR spectra are recorded using a Bruker UltraShield spectrometer operating at 400 MHz (1H) and 100 MHz (¹³C). Spectrum calibration is performed by adding tetramethyl silane (TMS) to the deuterated solvent as the internal reference. The calibration is obtained by setting the 0 to the TMS signal.

For fluorine 19, CFCl3 is used as an external reference. Chemical displacements are reported in parts per million (ppm) and coupling constants are given in Hertz (Hz). Abbreviations for the multiplicity of proton and carbon signals are: s singlet, d doublet, dd doublet of doublet, dt doublet of triplets, ddt doublet of triplets, t triplet, tt triplet of triplets, q, quintet, m multiplet.

### Mass Spectrometry (SM):

Mass spectra are performed using a Bruker Q-TOF maXis coupled to a Dionex Ultimate 3000 RSLC chain used in FIA (Flow Injection Analysis=without column) with an ACN/H2O+0.1% formic acid mixture 65/35 to 200 μL/min as the solvent. The injection volume is 0.2 μL. Most of the time, the analyses are performed in positive mode with the ESI source (Electrospray Ionisation).

### Preparation of Samples:

The samples are taken at a concentration of about 1 mg/mL with the solvent then diluted approximately 500 times (≈2 ng/μL) in methanol (sometimes another solvent more appropriate according to the structure of the compound to be analyzed: water, acetonitrile . . . ). If the signal obtained is insufficient, the sample concentration is increased.

### Melting Point Measurement:

Melting points are measured using a STUART SMP3.

### High Performance Liquid Chromatography (HPLC):

HPLC analyses are performed on a Waters analytical HPLC system (Waters Delta 600 Multisolvent pump, Waters 600 system controller, Rheodyne 7725i injector with a 20 μl sample loop) controlled with Empower software and equipped with appropriate analytical column. The detection is carried out with a UV detector with photodiode strip (Waters 2996) and/or a refractometer.

### 1-[4-[(6,7-dimethoxy-1-isoquinolyl)methyl]phenyl]pyrrolidin-2-one (1)

The above compound was prepared according to Scheme 3 and under the specific conditions below:

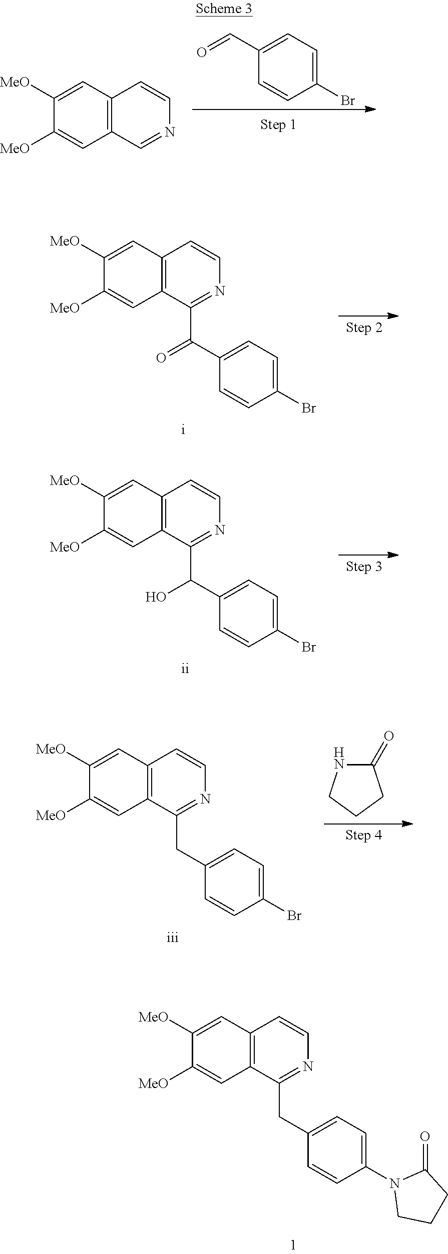

To a solution of 6,7-dimethoxyisoquinoline (2 g, 10.57 mmol) in dichloroethane (4 mL) was added trifluoroacetic acid (1.21 g, 10.57 mmol, 782.61 uL), 4-bromobenzaldehyde (5.87 g, 31.71 mmol) and 2-hydroperoxy-2-methyl-propane (5 M, 6.34 mL). The mixture was stirred at 110° C. for 16 hrs. LCMS showed 50% of the starting materials was remained, and desired Ms was detected. The reaction mixture was quenched with water (40 mL) and extracted with ethyl acetate (40 mL). The organic phase was dried over Na₂SO₄, filtered and concentrated under reduced pressure to give a residue. The residue was purified by column chromatography (SiO2 Petroleum ether/Ethyl acetate=I/O to 20/1). The organic was concentrated under reduced pressure to give (4-bromophenyl)-6,7-dimethoxy-1-isoquinoline-methanone i (800 mg, 19.32% yield) as a light yellow solid.

¹H NMR (400 MHz DMSO-d₆): δ 8.42 (d, J=5.38 Hz, 1H) 7.91 (d, J=5.50 Hz, 1H) 7.73-7.82 (m, 4H) 7.53 (d, J=7.25 Hz, 2H) 3.97 (s, 3H) 3.85 (s, 3H).

To a solution of i (600 mg, 1.53 mmol) in MeOH (6 mL) was added NaBH₄ (63.73 mg, 1.68 mmol) at 0° C. Then the mixture was stirred at 25° C. for 3 hrs. LCMS showed the starting materials was consumed, and desired Ms was detected. The reaction was cooled to 0° C. and quenched with ice water (20 mL). The solution was stirred at 25° C. for 10 minutes and then adjusted to pH=7 with 1 N HCl. The reaction solution was extracted with ethyl acetate (40 mL). The organic phase was dried over Na₂SO₄, filtered and concentrated under reduced pressure to give crude ii (460 mg, 76.25% yield) as gray solid.

¹H NMR (400 MHz DMSO-d₆): δ 8.31 (d, J=5.50 Hz, 1H) 7.59-7.66 (m, 2H) 7.44-7.52 (m, 2H) 7.39 (d, J=8.50 Hz, 2H) 7.34 (s, 1H) 6.42 (d, J=5.50 Hz, 1H) 6.33 (d, J=5.38 Hz, 1H) 3.89 (s, 3H) 3.80 (s, 3H).

To a solution of ii (460 mg, 1.17 mmol) in H₂SO₄ (2 mL) was added triethylsilane (1.36 g, 11.68 mmol, 1.87 mL) at 20° C. The mixture was stirred at 50° C. for 16 hrs. LCMS showed 33% of the starting materials was remained and desired Ms was detected. This reaction was poured into 50 mL ice water and adjusted to pH=8 with Na₂CO₃. The mixture was extracted with ethyl acetate (30 mL×3). The combined organic layers were dried over Na₂SO₄, filtered and concentrated under reduced pressure to give a residue. The residue was purified by Pre-TLC (SiO₂, Petroleum ether:Ethyl acetate=1:1). The mixture was filtered and concentrated under reduced pressure to give iii (220 mg, 47.33% yield,) as a white solid.

¹H NMR (400 MHz DMSO-d₆): δ 8.24 (d, J=5.63 Hz, 1H) 7.55 (d, J=5.63 Hz, 1H) 7.44-7.48 (m, 2H) 7.42-7.44 (m, 1H) 7.33 (s, 1H) 7.28 (d, J=8.38 Hz, 2H) 4.53 (s, 2H) 3.90 (s, 3H) 3.87 (s, 3H).

To a solution of iii (150 mg, 376.85 umol) in dioxane (1 mL) was added pyrrolidin-2-one (38.49 mg, 452.23 umol, 34.67 uL), Pd₂(dba)₃ (17.25 mg, 18.84 umol), Cs₂CO₃ (368.36 mg, 1.13 mmol) and Xantphos (21.81 mg, 37.69 umol) was degassed and purged with N₂ for 3 times. The reaction mixture was stirred at 110° C. for 12 hrs. LCMS showed the starting materials was consumed and desired Ms was detected. The reaction mixture was filtered. The filtrate was concentrated in vacuum to give crude product. The crude product was purified by prep-TLC (SiO₂, Petroleum ether:Ethyl acetate=0:1) to give (1) (70.4 mg, 50.72% yield) as a white solid. ¹H NMR (400 MHz CDCl3): δ 8.37 (d, J=5.63 Hz, 1H) 7.48-7.54 (m, 2H) 7.43 (d, J=5.75 Hz, 1H) 7.29 (d, J=3.63 Hz, 2H) 7.25-7.27 (m, 1H) 7.05 (s, 1H) 4.58 (s, 2H) 4.01 (s, 3H) 3.90 (s, 3H) 3.81 (t, J=7.07 Hz, 2H) 2.58 (t, J=8.07 Hz, 2H) 2.13 (m, 2H).

### 6,7-dimethoxy-N-(4-methylsulfonylphenyl)isoquinolin-1-amine (2)

The above compound was prepared according to general Scheme 1 above and under the specific conditions of Scheme 4 below:

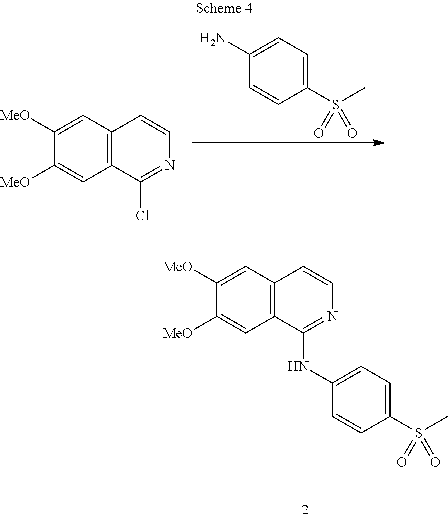 [SMILES: COc1cc2ccnc(Cl)c2cc1OC ; CS(=O)(=O)c1ccc(N)cc1 ; COc1cc2ccnc(Nc3ccc(S(C)(=O)=O)cc3)c2cc1OC]

To a solution of 1-chloro-6,7-dimethoxy-isoquinoline (1 g, 4.47 mmol, 1 eq) and 4-methylsulfonylaniline (765.54 mg, 4.47 mmol, 1 eq) in dioxane (20 mL) was added Cs₂CO₃ (2.91 g, 8.94 mmol, 2 eq) and SPhos (183.55 mg, 447.12 μmol, 0.1 eq) at 20° C., N₂ was bubbled through the mixture for 1 minute, then Pd₂(dba)₃ (204.72 mg, 223.56 μmol, 0.05 eq) was added under N₂, then N₂ was bubbled through the mixture for 1 minute, the reaction mixture was stirred at 100° C. for 16 hrs. TLC showed the two starting material was consumed and a new spot was detected. The reaction mixture was filtered through a pad of celite, and the cake was washed with methanol (3×20 mL), the filtrate was concentrated under reduced pressure. The residue was purified by column chromatography on silica gel eluted with methanol in dichloromethane from 0% to 4%, the elution fraction containing product was concentrated under reduced pressure, the residue was treated with ethyl acetate (10 mL), the solid was collected by filtration, and dried in high vacuum to give (1.27 g, 79.46% yield) of the target compound (2) as a white solid. ¹H NMR 400 MHz (d6-DMSO): δ=9.36 (s, 1H), 8.04 (d, J=8.9 Hz, 2H), 7.95 (d, J=5.6 Hz, 1H), 7.87-7.75 (m, 3H), 7.31 (s, 1H), 7.24 (d, J=5.8 Hz, 1H), 3.98 (s, 3H), 3.92 (s, 3H), 3.16 (s, 3H).

### 6-methoxy-1-(4-methylsulfonylanilino)isoquinolin-7-ol (3), 7-methoxy-1-(4-methylsulfonyl anilino)isoquinolin-6-ol (4) and 1-(4-methylsulfonylanilino)isoquinoline-6,7-diol (5)

The above compounds were prepared according to Scheme 5 and under the specific conditions below:

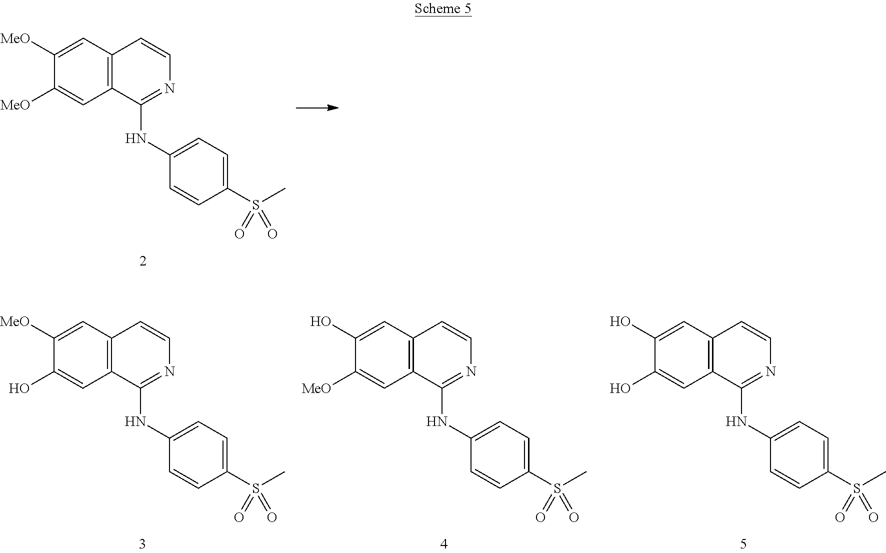 [SMILES: COc1cc2ccnc(Nc3ccc(S(C)(=O)=O)cc3)c2cc1OC ; COc1cc2ccnc(Nc3ccc(S(C)(=O)=O)cc3)c2cc1O ; COc1cc2c(Nc3ccc(S(C)(=O)=O)cc3)nccc2cc1O ; CS(=O)(=O)c1ccc(Nc2nccc3cc(O)c(O)cc23)cc1]

The mixture of compound 2 (1 g, 2.80 mmol) in 47% aq. HBr (20 mL) was stirred at 100° C. for 7 hrs. LCMS showed the starting materials was consumed, and desired de-methylation Ms was detected. The mixture was concentrated under reduced pressure to give crude product. The crude product was purified by Pre-HPLC (TFA condition) and lyophilized to give compounds (3), (4) and (5) (TFA salt). The product was respectively triturated in HCl (4 N)/MTBE (2 mL) and the solid was collected by filtration, the cake was washed with MTBE (2 mL) and dissolved in 10 mL water and 10 mL acetonitrile, then lyophilized to respectively give (3) (66.4 mg, 6.02% yield, HCl salt) as a white solid, (4) (36.7 mg, 3.4% yield, HCl salt) as a white solid and (5) (72.1 mg, 6.8% yield, HCl salt) as a white solid.

(3): ¹H NMR (400 MHz DMSO-d₆): δ 10.09-10.78 (m, 2H) 7.98 (br d, J=7.88 Hz, 2H) 7.91 (s, 1H) 7.81 (br d, J=7.75 Hz, 2H) 7.60-7.71 (m, 1H) 7.51 (br s, 1H) 7.38 (br d, J=6.50 Hz, 1H) 4.02 (s, 3H) 3.24 (s, 3H). (4): ¹H NMR (400 MHz DMSO-d₆): δ 10.75-11.27 (m, 1H) 8.13 (br s, 1H) 8.03 (br d, J=8.26 Hz, 2H) 7.83 (br d, J=8.25 Hz, 2H) 7.59 (br d, J=5.00 Hz, 1H) 7.32 (br d, J=6.50 Hz, 1H) 7.29 (s, 1H) 4.02 (s, 3H) 3.26 (s, 3H). (5): ¹H NMR (400 MHz DMSO-d₆): δ 12.71-13.50 (m, 1H) 11.14-11.51 (m, 1H) 10.51-10.94 (m, 1H) 10.02-10.44 (m, 1H) 8.00 (br d, J=8.50 Hz, 2H) 7.93 (s, 1H) 7.75 (br d, J=8.50 Hz, 2H) 7.53 (br d, J=6.50 Hz, 1H) 7.34 (d, J=6.63 Hz, 1H) 7.31 (s, 1H) 3.25 (s, 3H).

### 6-ethoxy-1-(4-methylsulfonylanilino)isoquinolin-7-ol, (6), 7-ethoxy-1-(4-methylsulfonyl anilino)isoquinolin-6-ol (7) and 6,7-diethoxy-N-(4-methylsulfonylphenyl)isoquinolin-1-amine (8)

The above compounds were prepared according to Scheme 6 and under the specific conditions below:

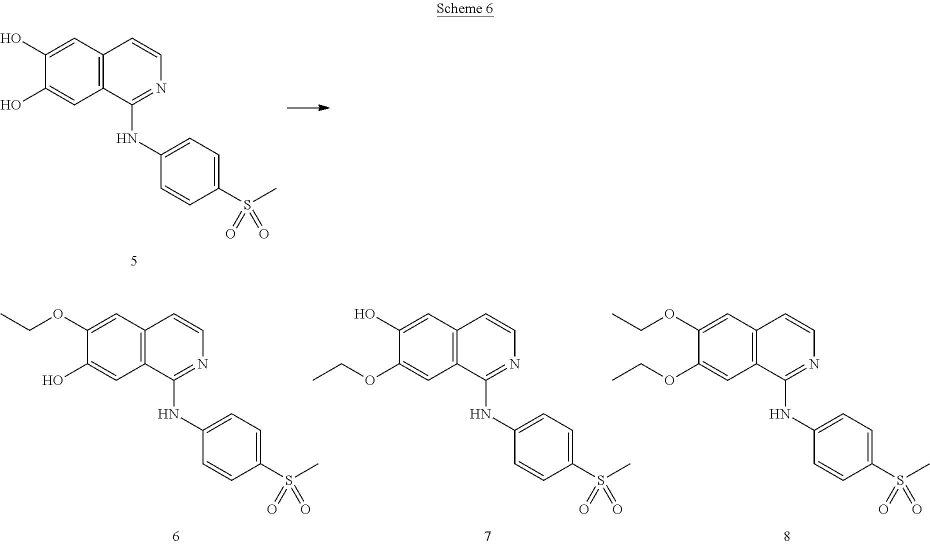 [SMILES: CS(=O)(=O)c1ccc(Nc2nccc3cc(O)c(O)cc23)cc1 ; CCOc1cc2ccnc(Nc3ccc(S(C)(=O)=O)cc3)c2cc1O ; CCOc1cc2c(Nc3ccc(S(C)(=O)=O)cc3)nccc2cc1O ; CCOc1cc2ccnc(Nc3ccc(S(C)(=O)=O)cc3)c2cc1OCC]

To a solution of iodoethane (240.78 mg, 1.54 mmol) in DMF (12 mL) was added K₂CO₃ (640.10 mg, 4.63 mmol) and compound (5) (600 mg, 1.54 mmol) at 0° C. Then the mixture was stirred at 60° C. for 3 hrs. LCMS showed all the starting material were consumed, desired Ms was detected. The reaction mixture was concentrated under reduced pressure to give crude product. The crude product was purified by pre-HPLC. The mobile phase was concentrated under high vacuum to give (7) (54.5 mg, 9.33% yield) as a brown solid and (6) (73.6 mg, 13.04% yield) as a brown solid and (8) (22.1 mg, 3.64% yield) as a brown solid. (6): ¹H NMR (400 MHz DMSO-d₆): δ 9.52 (s, 1H) 9.35 (s, 1H) 8.03 (br d, J=8.76 Hz, 2H) 7.89 (d, J=5.63 Hz, 1H) 7.79 (br d, J=9.26 Hz, 3H) 7.27 (s, 1H) 7.19 (br d, J=5.63 Hz, 1H) 4.21 (m, 2H) 3.14 (s, 3H) 1.44 (br t, J=6.88 Hz, 3H). (7): ¹H NMR (400 MHz DMSO-d₆): δ 10.05 (br s, 1H) 9.31 (br s, 1H) 8.04 (br d, J=8.63 Hz, 2H) 7.74-7.92 (m, 4H) 7.06-7.16 (m, 2H) 4.25 (m, 2H) 3.16 (s, 3H) 1.45 (br t, J=6.88 Hz, 3H). (8): ¹H NMR (400 MHz DMSO-d₆): δ 9.32 (s, 1H) 8.04 (d, J=8.88 Hz, 2H) 7.94 (d, J=5.63 Hz, 1H) 7.82 (d, J=8.88 Hz, 2H) 7.78 (s, 1H) 7.29 (s, 1H) 7.21 (d, J=5.63 Hz, 1H) 4.22 (m, 4H) 3.16 (s, 3H) 1.43 (m, 6H)—

### 1-[4-[(6,7-dimethoxy-1-isoquinolyl)amino]phenyl]pyrrolidin-2-one (9)

The above compound was prepared according to general Scheme 1 and under the specific conditions of Scheme 7 below:

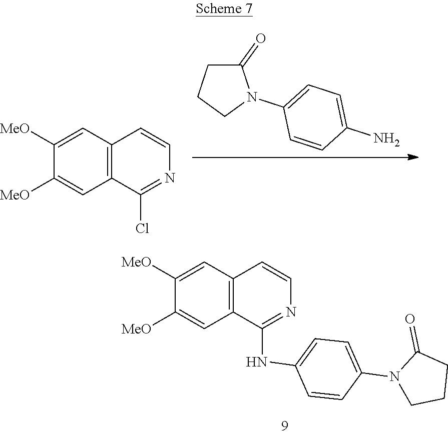

To a solution of 1-chloro-6,7-dimethoxy-isoquinoline (150 mg, 670.68 μmol, 1 eq) and 1-(4-aminophenyl) pyrrolidin-2-one (118.18 mg, 670.68 μmol, 1 eq) in dioxane (3 mL) was added Cs₂CO₃ (437.04 mg, 1.34 mmol, 2 eq) and SPhos (27.53 mg, 67.07 μmol, 0.1 eq) at 20° C., N₂ was bubbled through the mixture for 1 minute, then Pd₂(dba)3 (30.71 mg, 33.53 μmol, 0.05 eq) was added under N₂, then N₂ was bubbled through the mixture for 1 minute, the reaction mixture was stirred at 100° C. for 16 hrs. TLC showed the two starting material was consumed and a new spot was detected. The reaction mixture was filtered through a pad of celite, and the cake was washed with methanol (3×5 mL), the filtrate was concentrated under reduced pressure. The residue was purified by prep-HPLC under neutral condition, and lyophilized to give (9) (130.7 mg, 52.77% yield) as a white solid. ¹H NMR (400 MHz DMSO-d₆): δ=8.88 (s, 1H), 7.84-7.75 (m, 4H), 7.61-7.57 (m, 2H), 7.23 (s, 1H), 7.05 (d, J=5.7 Hz, 1H), 3.96 (s, 3H), 3.90 (s, 3H), 3.84 (t, J=7.0 Hz, 2H), 2.49-2.46 (m, 2H), 2.12-2.01 (m, 2H).

### N-(4-methylsulfonylphenyl)-6-vinyloxy-isoquinolin-1-amine (10)

The above compound was prepared according to general Scheme 1 and under the specific conditions below of Schemes 8 to 12:

### Step 1:

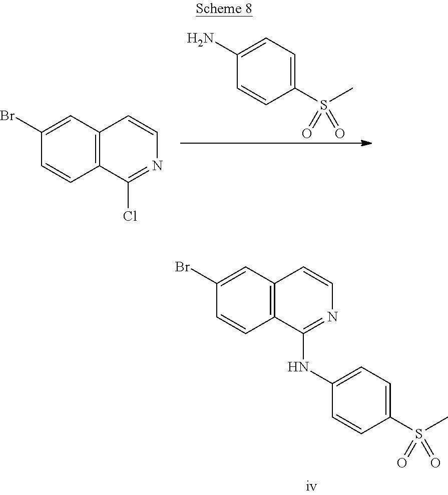 [SMILES: Clc1nccc2cc(Br)ccc12 ; CS(=O)(=O)c1ccc(N)cc1 ; CS(=O)(=O)c1ccc(Nc2nccc3cc(Br)ccc23)cc1]

To a suspension of 6-bromo-1-chloroisoquinoline (3 g, 12.37 mmol, 1 eq) and 4-methylsulfonylaniline (2.12 g, 12.37 mmol, 1 eq) in i-PrOH (60 mL) was added HCl/dioxane (6 M, 3.09 mL, 1.5 eq) at 20° C., the reaction mixture was stirred at 90° C. for 16 hrs. LCMS showed most of starting material was consumed, product with desired MS was detected. The reaction mixture was filtered, the cake was washed with i-PrOH (10 mL), then suspended in ethyl acetate (30 mL), cooled to 0° C., and adjusted to pH 8 with saturated aqueous NaHCO₃, the two phase was separated, and the aqueous phase was extracted with ethyl acetate (2×20 mL), the combined organic layer was wash with saturated brine (20 mL), dried under reduced pressure. The residue was treated with a mixture of ethanol and water (15 mL, 1:1), the solid was collected by filtration, this process was repeated for 2 times to get pure product. The cake was dried in high vacuum to give 6-bromo-N-(4-methylsulfonylphenyl)isoquinolin-1-amine iv (2 g, 42.85% yield) as a white solid. ¹H NMR (400 MHz DMSO-d₆): δ=9.72 (s, 1H), 8.52 (d, J=9.0 Hz, 1H), 8.19 (d, J=1.8 Hz, 1H), 8.16-8.10 (m, 3H), 7.88-7.81 (m, 3H), 7.32 (d, J=5.7 Hz, 1H), 3.17 (s, 3H).

### Step 2:

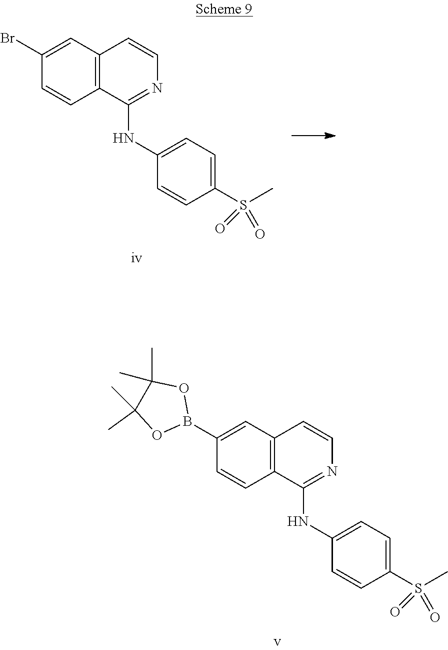

To a solution of 6-bromo-N-(4-methylsulfonylphenyl)isoquinolin-1-amine iv (300 mg, 795.22 umol, 1 eq) and 4,4,5,5-tetramethyl-2-(4,4,5,5-tetramethyl-1,3,2-dioxaborolan-2-yl)-1,3,2-dioxaborolane (242.32 mg, 954.26 μmol, 1.2 eq) in dioxane (6 mL) was added KOAc (156.09 mg, 1.59 mmol, 2 eq) at 20° C., N₂ was bubbled through the mixture for 1 minute, then Pd(dppf)Cl₂ (64.94 mg, 79.52 μmol, 0.1 eq) was added under N₂, then N₂ was bubbled through the mixture for 1 minute, the reaction mixture was stirred at 80° C. for 12 hrs. LCMS showed desired product (boric acid) was detected. The reaction mixture was filtered through a pad of celite, the cake was washed with ethyl acetate (2×5 mL), the filtrate was concentrated under reduced pressure. The crude product was purified by column chromatography on silica gel eluted with ethyl acetate in petroleum ether from 0% to 50% to give N-(4-methylsulfonylphenyl)-6-(4,4,5,5-tetramethyl-1,3,2-dioxaborolan-2-yl)isoquinolin-1-amine v (330 mg, 97.80% yield) as a white foam. ¹H NMR (400 MHz DMSO-d₆): δ=9.69 (s, 1H), 8.55 (d, J=8.5 Hz, 1H), 8.25 (s, 1H), 8.20-8.08 (m, 3H), 7.90-7.80 (m, 3H), 7.44 (d, J=5.8 Hz, 1H), 3.17 (s, 3H), 1.35 (s, 12H).

### Step 3:

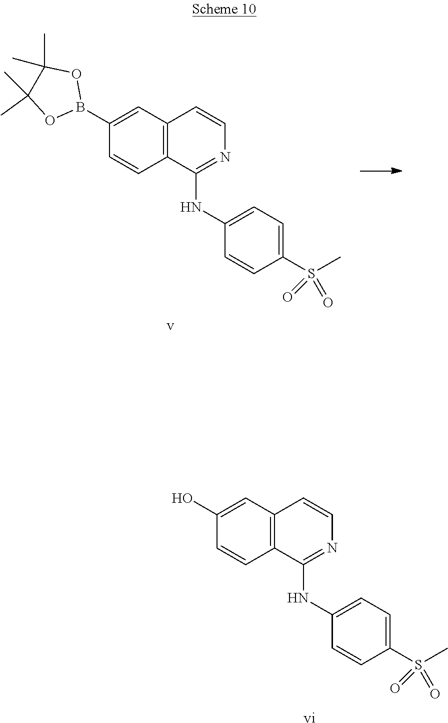

To a solution of N-(4-methylsulfonylphenyl)-6-(4,4,5,5-tetramethyl-1,3,2-dioxaborolan-2-yl)isoquinolin-1-amine v (330 mg, 777.72 μmol, 1 eq) in THE (6 mL) and Water (3 mL) was added sodium 3-oxidodioxaborirane; tetrahydrate (358.98 mg, 2.33 mmol, 3 eq) in portions at 20° C., the reaction mixture was stirred at 20° C. for 12 hrs. LCMS showed desired product was detected. Cold water (18 mL) was added to the reaction mixture, the resulting solid was collected by filtration, the cake was washed with water (2 mL), and dried in high vacuum to give 1-(4-methylsulfonylanilino)isoquinolin-6-ol vi (200 mg, 81.81% yield) as a white solid. vi: ¹H NMR (400 MHz DMSO-d₆): δ=10.31 (br s, 1H), 9.48 (s, 1H), 8.40 (d, J=9.0 Hz, 1H), 8.11 (d, J=8.9 Hz, 2H), 7.95 (d, J=5.8 Hz, 1H), 7.82 (d, J=8.6 Hz, 2H), 7.14 (d, J=5.9 Hz, 2H), 7.07 (d, J=2.3 Hz, 1H), 3.16 (s, 3H).

### Step 4:

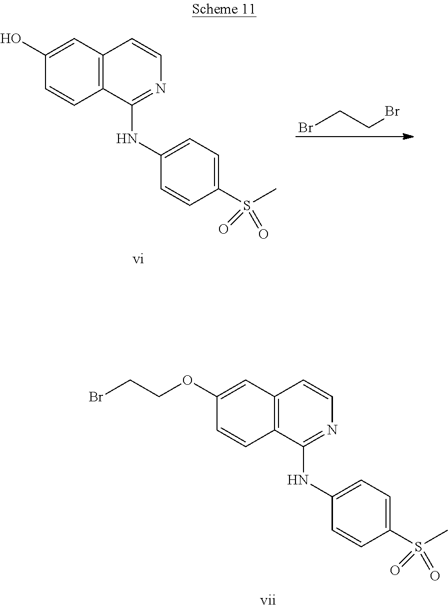

To a solution of 1-(4-methylsulfonylanilino)isoquinolin-6-ol vi (180 mg, 572.59 μmol, 1 eq) in DMF (5 mL) was added 1,2-dibromoethane (537.84 mg, 2.86 mmol, 216.00 μL, 5 eq) and K₂CO₃ (158.27 mg, 1.15 mmol, 2 eq) at 20° C., the reaction mixture was stirred at 60° C. for 12 hr. LCMS showed desired product was detected. The reaction mixture was filtered to remove salt, the cake was washed with ethyl acetate (2×10 mL), the filtrate was washed with brine (5 mL), dried over anhydrous Na₂SO₄, filtered, concentrated under reduced pressure. The residue was treated with petroleum ether and ethyl acetate (5 mL, 5:1), the solid was collected by filtration, washed with petroleum ether and ethyl acetate (2 mL, 5:1), dried in high vacuum to give 6-(2-bromoethoxy)-N-(4-methylsulfonylphenyl)isoquinolin-1-amine vii (120 mg, 49.74% yield) as a white solid. vii: ¹H NMR (400 MHz CDCL3): δ=8.14 (d, J=5.8 Hz, 1H), 7.99-7.82 (m, 5H), 7.34-7.29 (m, 1H), 7.27 (br d, J=2.4 Hz, 1H), 7.21 (br d, J=5.6 Hz, 1H), 7.10 (d, J=2.4 Hz, 1H), 4.48 (t, J=6.2 Hz, 2H), 3.75 (t, J=6.1 Hz, 2H), 3.07 (s, 3H).

### Step 5:

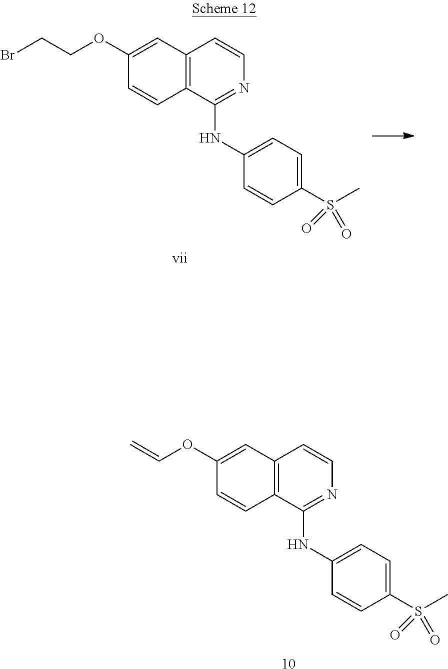

To a solution of 6-(2-bromoethoxy)-N-(4-methylsulfonylphenyl)isoquinolin-1-amine vii (110 mg, 261.09 μmol, 1 eq) in DMSO (3 mL) was added t-BuOK (1 M, 652.73 uL, 2.5 eq) at 20° C., the reaction mixture was stirred at 20° C. for 1 hr. LCMS showed the starting material was consumed and desired product was detected. The reaction was quenched with water (10 mL), extracted with ethyl acetate (3×5 mL), washed with water (3 mL), dried over anhydrous Na₂SO₄, filtered, concentrated under reduced pressure. The residue was treated with petroleum ether and ethyl acetate (5 mL, 5:1), the solid was collected by filtration, washed with petroleum ether and ethyl acetate (2 mL, 5:1), dried in high vacuum to give compound (10) (25 mg, 28.13% yield) as a white solid. ¹H NMR (400 MHz DMSO-d₆): δ=9.62 (s, 1H), 8.55 (d, J=9.3 Hz, 1H), 8.12 (d, J=9.0 Hz, 2H), 8.06 (d, J=5.8 Hz, 1H), 7.83 (d, J=8.9 Hz, 2H), 7.48 (d, J=2.5 Hz, 1H), 7.41 (dd, J=2.6, 9.1 Hz, 1H), 7.29 (d, J=5.8 Hz, 1H), 7.11 (dd, J=6.0, 13.5 Hz, 1H), 4.94 (dd, J=1.6, 13.5 Hz, 1H), 4.68 (dd, J=1.6, 6.0 Hz, 1H), 3.16 (s, 3H).

### 6-isopropoxy-N-(4-methylsulfonylphenyl)isoquinolin-1-amine (11)

The above compound was prepared according to Scheme 13 and under the specific conditions below:

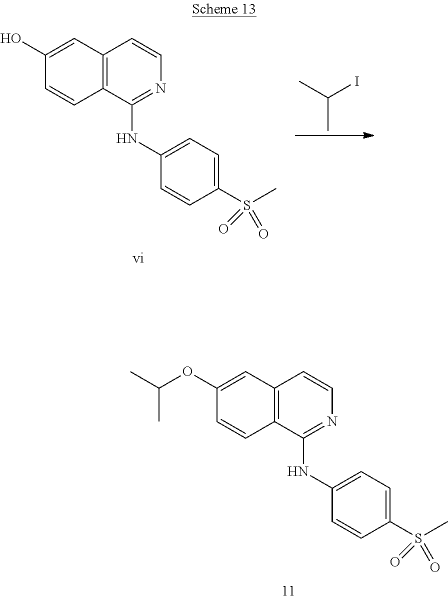

To a solution of 1-(4-methylsulfonylanilino)isoquinolin-6-ol vi (170 mg, 540.78 μmol, 1 eq) in DMF (5 mL) was added 2-iodopropane (459.64 mg, 2.70 mmol, 270.38 uL, 5 eq) and K₂CO₃ (149.48 mg, 1.08 mmol, 2 eq) at 20° C., the reaction mixture was stirred at 60° C. for 6 hr. LCMS showed desired product was detected. The reaction mixture was quenched with ice water (15 mL), the resulting solid was collected by filtration, the cake was dried in high vacuum, then treated with petroleum ether and ethyl acetate (3 mL, 2:1), the solid was collected by filtration, washed with petroleum ether and ethyl acetate (1 mL, 2:1), dried in high vacuum to give compound (11) (111.8 mg, 55.39% yield) as a white solid. ¹H NMR (400 MHz DMSO-d₆): δ=9.53 (s, 1H), 8.44 (d, J=9.3 Hz, 1H), 8.11 (d, J=8.9 Hz, 2H), 8.00 (d, J=5.8 Hz, 1H), 7.81 (d, J=8.9 Hz, 2H), 7.29 (d, J=2.4 Hz, 1H), 7.26-7.20 (m, 2H), 4.83 (spt, J=6.0 Hz, 1H), 3.15 (s, 3H), 1.35 (d, J=6.0 Hz, 6H).

### N-(4-methylsulfonylphenyl)-6-pyrimidin-2-yl-isoquinolin-1-amine (12)

The above compound was prepared according to Scheme 14 and under the specific conditions below:

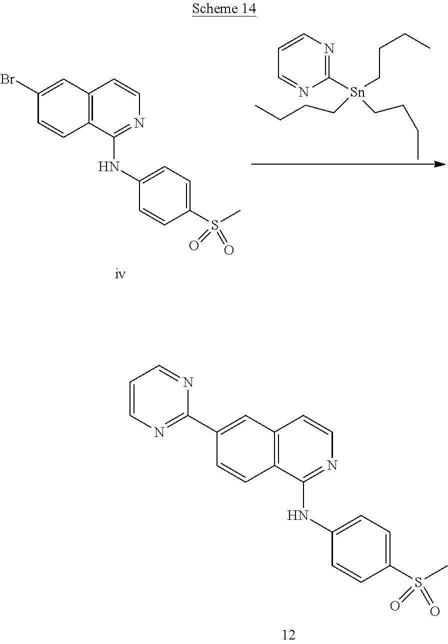 [SMILES: CS(=O)(=O)c1ccc(Nc2nccc3cc(Br)ccc23)cc1 ; CCC[CH2][Sn]([CH2]CCC)([CH2]CCC)[c]1ncccn1 ; CS(=O)(=O)c1ccc(Nc2nccc3cc(-c4ncccn4)ccc23)cc1]

To a solution of 6-bromo-N-(4-methylsulfonylphenyl)isoquinolin-1-amine iv (200 mg, 530.15 mol, 1 eq) and tributyl(pyrimidin-2-yl)stannane (293.54 mg, 795.22 μmol, 1.5 eq) in dioxane (8 mL) was added CsF (161.06 mg, 1.06 mmol, 39.09 μL, 2 eq) at 20° C., N₂ was bubbled through the mixture for 1 minute, then CuI (20.19 mg, 106.03 μmol, 0.2 eq) and Pd(PPh₃)₄ (61.26 mg, 53.02 μmol, 0.1 eq) was added under N₂, then N₂ was bubbled through the mixture for 1 minute, the reaction mixture was stirred at 100° C. for 12 hrs. LCMS showed desired product was detected. The reaction mixture was filtered through a pad of celite, the cake was washed with methanol (5 mL), the filtrate was concentrated under reduced pressure. The residue was purified prep-HPLC under neutral condition, and lyophilized to give compound (12) (75.8 mg, 37.37% yield) as a white solid. H NMR (400 MHz DMSO-d₆): δ=9.76 (s, 1H), 9.02 (d, J=4.9 Hz, 2H), 8.93 (d, J=1.4 Hz, 1H), 8.71 (d, J=8.9 Hz, 1H), 8.62 (dd, J=1.6, 8.9 Hz, 1H), 8.20-8.15 (m, 3H), 7.86 (d, J=8.9 Hz, 2H), 7.58-7.53 (m, 2H), 3.18 (s, 3H).

### N-(4-methylsulfonylphenyl)-6-pyrimidin-2-yl-isoquinolin-1-amine (13)

The above compound was prepared according to Schemes 15 to 18 and under the specific conditions below:

### Step 1:

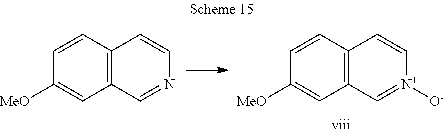 [SMILES: COc1ccc2ccncc2c1 ; COc1ccc2cc[n+]([O-])cc2c1]

To a solution of 7-methoxyisoquinoline (2 g, 12.56 mmol) in dichloromethane (20 mL) was added metachloroperbenzoic acid (3.06 g, 15.08 mmol) at 0° C. The mixture was stirred at 20° C. for 1 hr. LCMS showed the starting materials was consumed, and desired Ms was detected. The reaction was quenched with 4 N HCl in methyl tertiary butyl ether (5 mL), filtered and the filter cake was concentrated under reduced pressure to give the crude product. The crude product was triturated with methyl tertiary butyl ether (10 mL) at 20° C. for 10 minutes. The mixture was filtered, then the filter cake was concentrated under reduced pressure to give 7-methoxy-2-oxido-isoquinolin-2-ium viii (1.97 g, 85.03% yield) as a light yellow solid. ¹H NMR (400 MHz DMSO-d₆): δ 9.48 (s, 1H), 8.45 (dd, J=1.6, 7.0 Hz, 1H), 8.25 (d, J=7.0 Hz, 1H), 8.12 (d, J=9.0 Hz, 1H), 7.67 (d, J=2.1 Hz, 1H), 7.59 (dd, J=2.4, 8.9 Hz, 1H), 3.94 (s, 3H)

### Step 2:

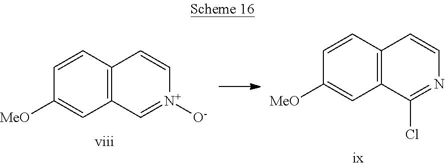 [SMILES: COc1ccc2cc[n+]([O-])cc2c1 ; COc1ccc2ccnc(Cl)c2c1]

A mixture of 7-methoxy-2-oxido-isoquinolin-2-ium viii (1.9 g, 10.30 mmol) in phosphorus oxychloride (20 mL) was stirred at 80° C. for 2 hrs. LCMS showed the starting materials was consumed, and desired Ms was detected. The reaction mixture was quenched by addition water 50 mL at 20° C. and extracted with ethyl acetate (40 mL×3). The combined organic layers were washed with brine (50 mL), dried over anhydrous sodium sulfate, filtered and concentrated under reduced pressure to give a residue. The residue was purified by silica gel chromatography (eluted with ethyl acetate in petroleum ether from 15% to 50%) to give 1-chloro-7-methoxy-isoquinoline ix (1.1 g, 52.38% yield) as a white solid. ¹H NMR (400 MHz DMSO-d₆): δ 8.18 (d, J=5.5 Hz, 1H), 8.02 (d, J=9.0 Hz, 1H), 7.84 (d, J=5.4 Hz, 1H), 7.54 (dd, J=2.5, 8.9 Hz, 1H), 7.49 (d, J=2.4 Hz, 1H), 3.96 (s, 3H)

### Step 3:

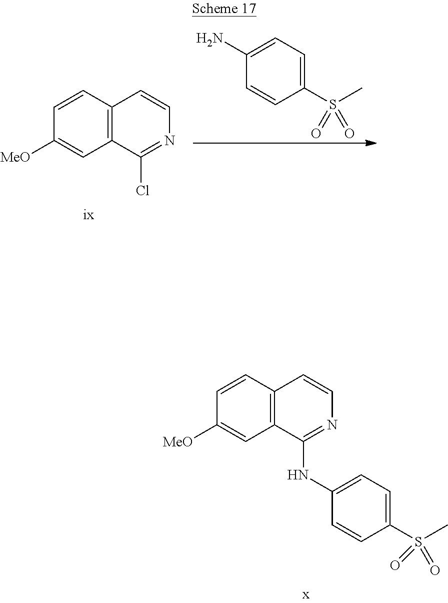 [SMILES: COc1ccc2ccnc(Cl)c2c1 ; CS(=O)(=O)c1ccc(N)cc1 ; COc1ccc2ccnc(Nc3ccc(S(C)(=O)=O)cc3)c2c1]

To a solution of 1-chloro-7-methoxy-isoquinoline ix (0.5 g, 2.45 mmol) in 1,4-dioxane (10 mL) was added 4-methylsulfonylaniline (420.02 mg, 2.45 mmol) and dicesium carbonate (1.60 g, 4.91 mmol) at 25° C. The flask was filled with N₂ and evacuated (3×). SPhos (100.71 mg, 245.31 umol) and Pd₂(dba)₃ (112.32 mg, 122.66 umol) were added to the mixture and the flask was filled with N₂ and evacuated (3×). The mixture was stirred at 110° C. for 12 hrs. LCMS showed all the starting materials were consumed and the desired Ms detected. The reaction mixture was filtered and the filtrate was concentrated under reduced pressure to give the crude product. The residue was triturated in ethyl acetate (1 mL) at 25° C. for 30 minutes and filtered. The filter cake was concentrated under reduced pressure to give 7-methoxy-N-(4-methylsulfonylphenyl)isoquinolin-1-amine x (0.5 g, 62.07% yield) as a gray solid. ¹H NMR (400 MHz DMSO-d₆): δ 9.73-9.35 (m, 1H), 8.07 (d, J=8.8 Hz, 2H), 7.96 (d, J=5.5 Hz, 1H), 7.91 (d, J=1.8 Hz, 1H), 7.81 (dd, J=2.9, 9.0 Hz, 3H), 7.40 (dd, J=2.1, 8.9 Hz, 1H), 7.25 (br d, J=5.4 Hz, 1H), 3.97 (s, 3H), 3.21-3.07 (m, 3H).

### Step 4:

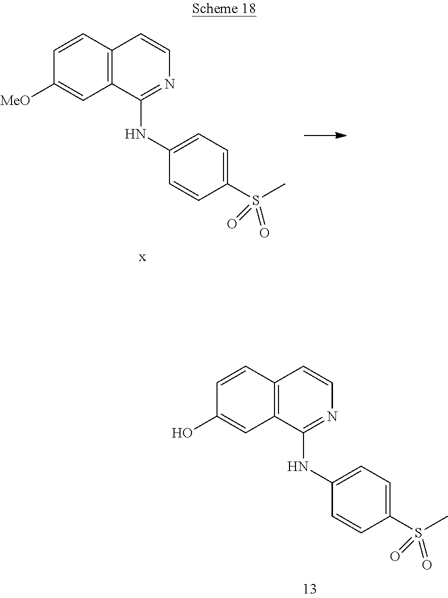 [SMILES: COc1ccc2ccnc(Nc3ccc(S(C)(=O)=O)cc3)c2c1 ; CS(=O)(=O)c1ccc(Nc2nccc3ccc(O)cc23)cc1]

To a solution of 7-methoxy-N-(4-methylsulfonylphenyl)isoquinolin-1-amine x (300 mg, 913.56 μmol) in AcOH (6 mL) was added HBr (335.99 mg, 1.37 mmol) at 20° C. The reaction solution was heated to 100° C. slowly and stirred for 5 hrs. LCMS showed all the starting materials were consumed and the desired Ms was detected. The reaction mixture was concentrated under reduced pressure and then adjusted to pH=8 with aq. NaHCO₃. The mixture was filtered and the filter cake was concentrated under reduced pressure to give the crude product. The crude product was purified by pre-HPLC and lyophilized to give 1-(4-methylsulfonylanilino)isoquinolin-7-ol (13) (112.3 mg, 39.10% yield) as a white solid. ¹H NMR (400 MHz DMSO-d₆): δ 10.04 (br s, 1H), 9.43 (s, 1H), 8.08 (br d, J=8.5 Hz, 2H), 7.91 (br d, J=5.5 Hz, 1H), 7.85-7.71 (m, 4H), 7.34 (br d, J=8.8 Hz, 1H), 7.25 (br d, J=5.5 Hz, 1H), 3.15 (s, 3H)

### N-[1-(4-methylsulfonylanilino)-7-isoquinolyl]acetamide (14) and N7-ethyl-N1-(4-methyl sulfonylphenyl)isoquinoline-1,7-diamine (15)

The above compounds were prepared according to general Scheme 1 and under the specific conditions of Schemes 19 to 20 below:

### Step 1:

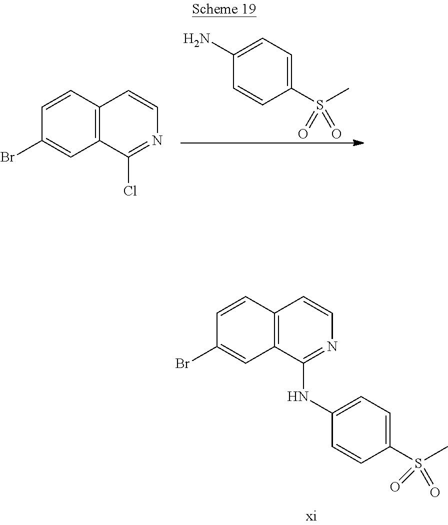 [SMILES: Clc1nccc2ccc(Br)cc12 ; CS(=O)(=O)c1ccc(N)cc1 ; CS(=O)(=O)c1ccc(Nc2nccc3ccc(Br)cc23)cc1]

To a mixture of 7-bromo-1-chloro-isoquinoline (2.10 g, 12.27 mmol) in isopropanol (60 mL) was added 4-methylsulfonylaniline (3 g, 12.27 mmol) and 6 M HCl/dioxane (3.07 mL) to the mixture at 20° C. The mixture was stirred at 90° C. for 12 hrs. LCMS showed the starting materials was consumed, and desired Ms was detected. The reaction mixture was filtered, the filter cake was washed with isopropanol (10 mL), then suspended in ethyl acetate (30 mL), cooled to 0° C., and adjusted to pH 8 with saturated aqueous NaHCO₃. The mixture was extracted with ethyl acetate (2×50 mL). The combined organic phases were washed with brine (30 mL), dried over anhydrous sodium sulfate, filtered and concentrated under reduced pressure to give the crude product. The residue was triturated with ethanol and water (15 mL, 1:1) at 80° C. for 0.5 hr, the solid was collected by filtration, this process was repeated for 2 times to give 7-bromo-N-(4-methylsulfonylphenyl)isoquinolin-1-amine xi (1.4 g, 27.22% yield) as a light yellow solid. ¹H NMR (400 MHz DMSO-d₆): δ 9.68 (s, 1H), 8.86 (s, 1H), 8.18-8.10 (m, 3H), 7.93-7.81 (m, 4H), 7.36 (d, J=5.6 Hz, 1H), 3.17 (s, 3H).

### N-[1-(4-methylsulfonylanilino)-7-isoquinolyl]acetamide (14)

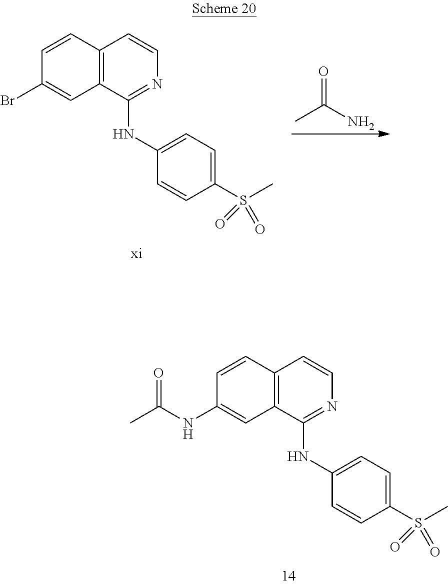

To a solution of 7-bromo-N-(4-methylsulfonylphenyl)isoquinolin-1-amine xi (1.4 g, 3.34 mmol) in 1,4-dioxane (28 mL) was added acetamide (256.46 mg, 4.34 mmol) and K₃PO₄ (2.13 g, 10.02 mmol) at 25° C. The flask was filled with N₂ and evacuated (3×). Pd₂(dba)₃ (152.92 mg, 167.00 μmol) and Xantphos (193.25 mg, 333.99 μmol,) were added to the mixture, and the flask was filled with N₂ and evacuated (3×). The mixture was stirred at 100° C. for 12 hrs. LCMS showed all the starting materials were consumed and the desired Ms was detected. The reaction mixture was filtered, the filtrate was concentrated under reduced pressure to give the residue. The residue was purified by silica gel chromatography (eluted with ethyl acetate in petroleum ether from 0% to 50%) to give N-[1-(4-methylsulfonylanilino)-7-isoquinolyl]acetamide (14) (0.75 g, 60.02% yield) as a light yellow solid. ¹H NMR (400 MHz DMSO-d₆): δ 10.29 (s, 1H), 9.63 (s, 1H), 8.60 (s, 1H), 8.04-7.93 (m, 3H), 7.89-7.78 (m, 4H), 7.32 (d, J=5.7 Hz, 1H), 3.16 (s, 3H), 2.12 (s, 3H).

### N7-ethyl-N1-(4-methylsulfonylphenyl)isoquinoline-1,7-diamine (15)

The above compound was prepared according to Scheme 21 and under the specific conditions below:

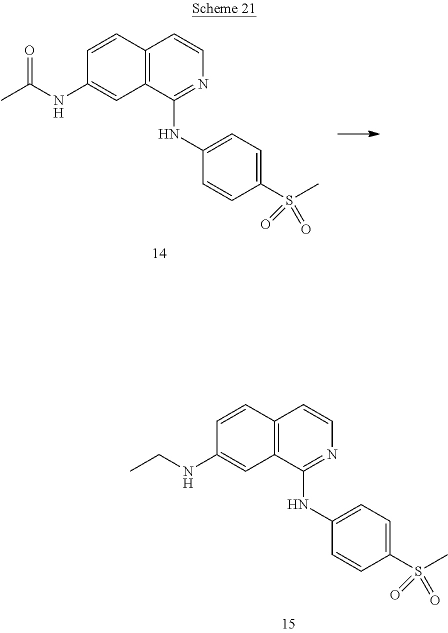

To a solution of N-[1-(4-methylsulfonylanilino)-7-isoquinolyl]acetamide (14) (300 mg, 801.89 mol) in tetrahydrofuran (6 mL) at 25° C. The flask was filled with N₂ and evacuated (3×). Then the reaction mixture was cooled to 0° C. and BH₃-Me₂S (10 M, 160.38 μL) was added to the reaction mixture. The mixture was stirred at 0° C. for 30 minutes. Then the reaction mixture was warmed to 20° C. and stirred for 30 mixtures and then slowly heated 60° C. stirred for 12 hrs. LCMS showed all the starting materials were consumed and the desired Ms was detected. The residue was quenched with methyl alcohol (5 mL) and stirred at 20° C. for 30 mixtures. Then the mixture was concentrated under reduced pressure to give the crude product. The crude product was purified by prep-HPLC and lyophilized to give N7-ethyl-N1-(4-methylsulfonylphenyl)isoquinoline-1,7-diamine (15) (76.2 mg, 27.41% yield) as a yellow solid. ¹H NMR (400 MHz DMSO-d₆): δ 10.29 (s, 1H), 9.63 (s, 1H), 8.60 (s, 1H), 8.04-7.93 (m, 3H), 7.89-7.78 (m, 4H), 7.32 (d, J=5.7 Hz, 1H), 3.16 (s, 3H), 2.12 (s, 3H).

### Intermediate 7-bromo-6-methoxy-N-(4-methylsulfonylphenyl)isoquinolin-1-amine XXVII

### Step 1:

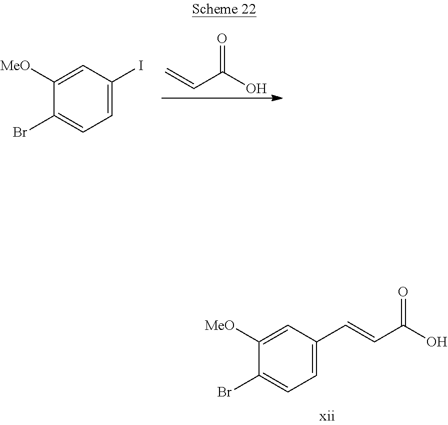

To a solution of 1-bromo-4-iodo-2-methoxy-benzene (10 g, 31.96 mmol) and acrylic acid (2.86 g, 39.63 mmol, 2.72 mL) in MeCN (30 mL) was added TEA (8.08 g, 79.89 mmol) and Pd(OAc)₂ (215.23 mg, 958.68 μmol) at 20° C. The mixture was stirred at 90° C. for 1 hour under N₂ atmosphere. TLC indicated starting material was consumed completely and one new spot formed. The reaction cooled to 20° C. and poured into ice aqueous solution of HCl (50 mL, 1N). After stirring for 10 minutes, the mixture was filtered to give crude product. The crude product was triturated with ethanol:hexane (50 mL)=1:1 at 20° C. and stirred for 20 minutes, then the mixture was filtered, the filter cake was dried by high vacuum to give (E)-3-(4-bromo-3-methoxy-phenyl)prop-2-enoic acid xii (6 g, 70.11% yield) as a gray solid.

### Step 2:

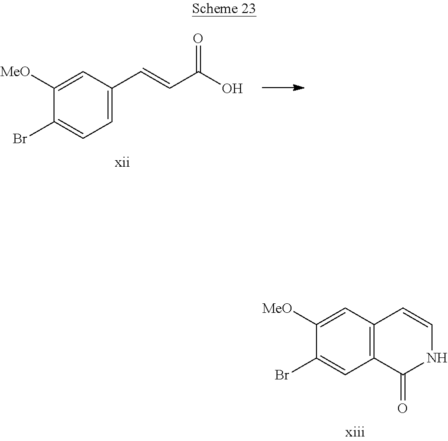 [SMILES: COc1cc(/C=C/C(=O)O)ccc1Br ; COc1cc2cc[nH]c(=O)c2cc1Br]

To a solution of (E)-3-(4-bromo-3-methoxy-phenyl)prop-2-enoic acid xii (20 g, 71.57 mmol, 92% purity, 1 eq) in toluene (160 mL) was added DPPA (19.70 g, 71.57 mmol, 15.51 mL, 1 eq) and TEA (10.14 g, 100.20 mmol, 13.95 mL, 1.4 eq) at 20° C. The mixture was stirred at 20° C. for 1 hr. TLC indicated starting material was consumed completely and one new spot formed. The mixture was filtered through a pad of silica and eluted with 1500 mL of toluene, filtrate was added diphenyl ether (150 mL), then the mixture was concentrated under reduced pressure to give crude product (about 20 g) in diphenyl ether (150 mL). Then the solution of (E)-3-(4-bromo-3-methoxy-phenyl) prop-2-enoyl azide (20.19 g, 71.57 mmol) in diphenyl ether (300 mL) was stirred at 230° C. for 1 hour. TLC indicated starting material was consumed completely and one new spot formed. The reaction mixture was cool to 20° C., then the mixture was added petroleum ether (500 mL), solid was precipitate out after 10 minutes, filtered to give crude product. The crude product was triturated with petroleum ether/ethyl acetate=5:1 and stirred for 20 minutes, then the mixture was filtered, the filter cake was dried by high vacuum to give 7-bromo-6-methoxy-isoquinolin-1-one xiii (7.5 g, 23.61 mmol, yield 32.99%, purity 80%) was obtained as a brown solid.

### Step 3:

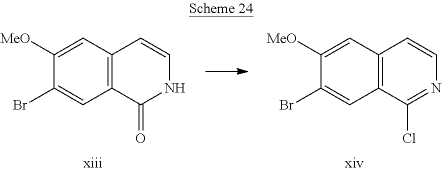 [SMILES: COc1cc2cc[nH]c(=O)c2cc1Br ; COc1cc2ccnc(Cl)c2cc1Br]

A solution of 7-bromo-6-methoxy-isoquinolin-1-one xiii (6 g, 13.22 mmol) in POCl₃ (60 mL) was stirred at 100° C. for 2 hours. LC-MS showed starting material as consumed completely and desired mass was detected. The reaction mixture was concentrated under reduced pressure to remove solvent. The residue was diluted with ethyl acetate (200 mL), the mixture was adjusted to pH=9 with saturated NaHCO₃ aqueous solution, then the mixture extracted with ethyl acetate (2×100 mL). The combined organic phase was washed with water and brine (2×100 mL), dried over Na₂SO₄, filtered and concentrated under reduced pressure to give crude product. The crude product was chromatographed on silica gel (petroleum ether/ethyl acetate=1:0 to 5:1) to give 7-bromo-1-chloro-6-methoxy-isoquinoline xiv (1.5 g, 34.13% yield) as a light-yellow solid.

### Step 4:

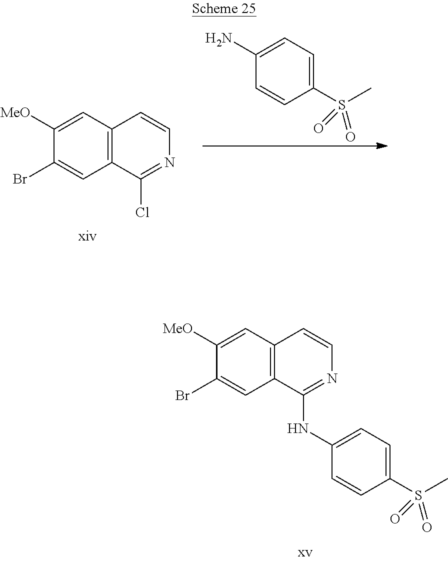 [SMILES: COc1cc2ccnc(Cl)c2cc1Br ; CS(=O)(=O)c1ccc(N)cc1 ; COc1cc2ccnc(Nc3ccc(S(C)(=O)=O)cc3)c2cc1Br]

To a solution of 7-bromo-1-chloro-6-methoxy-isoquinoline xiv (1.50 g, 4.51 mmol) in i-PrOH (30 mL) was added 4-methylsulfonylaniline (927.31 mg, 5.42 mmol) and HCl/dioxane (6 M, 1.13 mL) at 20° C., then the mixture was stirred at 90° C. for 12 hours. LCMS indicated 24% of starting material xiv was remained, and desired mass was detected. Then the mixture was cooled to 20° C., HCl/dioxane (6 M, 376.11 μL) was added to the mixture. The mixture was stirred at 100° C. for 4 hours. The reaction mixture was filtered, the filter cake was washed with i-PrOH (30 mL), then suspended in ethyl acetate (20 mL), cooled to 0° C., and adjusted to pH 8 with saturated aqueous NaHCO₃, the two phases were separated, and the aqueous phase was extracted with ethyl acetate (2×30 mL). The combined organic layer was wash with saturated brine (20 mL), dried under reduced pressure to give residue. The residue was treated with a mixture solvents of ethanol:water=1:1 (20 mL), the solid was collected by filtration, this process was repeated for 2 times to get 7-bromo-6-methoxy-N-(4-methylsulfonylphenyl)isoquinolin-1-amine xv (1.4 g, 53.31% yield) as a light yellow solid. ¹H NMR (400 MHz DMSO-d₆): δ9.60 (br s, 1H), 8.89 (s, 1H), 8.23-8.02 (m, 3H), 7.84 (br d, J=8.6 Hz, 2H), 7.45 (s, 1H), 7.28 (br d, J=5.6 Hz, 1H), 4.00 (s, 3H), 3.16 (s, 3H).

### 6-methoxy-N-(4-methylsulfonylphenyl)-7-vinyl-isoquinolin-1-amine (16)

The above compound was prepared according to Scheme 26 and under the specific conditions below:

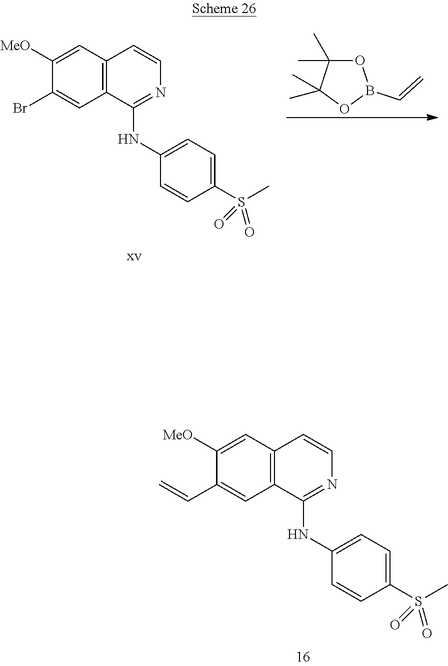 [SMILES: COc1cc2ccnc(Nc3ccc(S(C)(=O)=O)cc3)c2cc1Br ; C=CB1OC(C)(C)C(C)(C)O1 ; C=Cc1cc2c(Nc3ccc(S(C)(=O)=O)cc3)nccc2cc1OC]

To a solution of intermediate 7-bromo-6-methoxy-N-(4-methylsulfonylphenyl)isoquinolin-1-amine xv (0.8 g, 1.37 mmol) in THE (5 mL) and H₂O (2.5 mL) was added K₃PO₄ (583.72 mg, 2.75 mmol) and 4,4,5,5-tetramethyl-2-vinyl-1,3,2-dioxaborolane (254.12 mg, 1.65 mmol) at 20° C., then Pd(dppf)Cl₂ (112.29 mg, 137.50 μmol) was added in one portion at 20° C. The resulting mixture was stirred at 80° C. for 12 hours under N₂ atmosphere. LC-MS showed starting material was consumed completely and one main peak with desired mass was detected. The mixture diluted with water 20 mL at 20° C., then filtered and the filtrate was extracted with ethyl acetate (3×10 mL). The combined organic phase was washed with brine (2×20 mL), dried over anhydrous Na₂SO₄, filtered and concentrated under vacuum to give crude product. The crude product was chromatographed on silica gel (petroleum ether/tetrahydrofuran 80:20-70:30) to give 6-methoxy-N-(4-methylsulfonylphenyl)-7-vinyl-isoquinolin-1-amine (16) (0.4 g, 65.67% yield) as a light yellow solid. ¹H NMR (400 MHz DMSO-d₆): δ=9.60 (s, 1H), 8.63 (s, 1H), 8.11 (d, J=8.9 Hz, 2H), 8.00 (d, J=5.8 Hz, 1H), 7.84 (d, J=8.9 Hz, 2H), 7.32 (s, 1H), 7.24 (d, J=5.8 Hz, 1H), 7.09 (dd, J=11.2, 17.7 Hz, 1H), 6.12 (dd, J=1.5, 17.6 Hz, 1H), 5.53-5.38 (m, 1H), 3.97 (s, 3H), 3.17 (s, 3H).

### 7-ethyl-6-methoxy-N-(4-methylsulfonylphenyl)isoquinolin-1-amine (17)

The above compound was prepared according to Scheme 27 and under the specific conditions below:

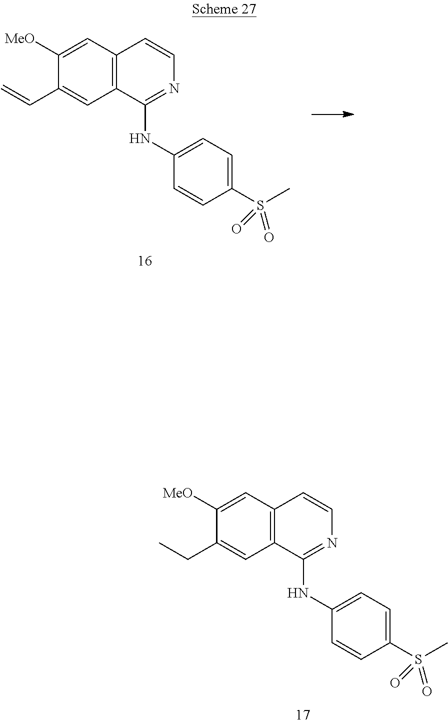

To a solution of 6-methoxy-N-(4-methylsulfonylphenyl)-7-vinyl-isoquinolin-1-amine 16 (400 mg, 902.88 μmol) in THE (8 mL) was added wet Pd/C (0.1 g, 90.29 μmol) under N₂ atmosphere. The suspension was degassed and purged with H₂ for 3 times. The mixture was stirred under H₂ (15 Psi) at 20° C. for 1 hour. LC-MS showed starting material was consumed completely and one main peak with desired Mass was detected. The reaction solution was filtered through the celite, the filtrate was concentrated to get crude product. The crude product was chromatographed on silica gel (petrolum ether/tetrahydrofuran=77:23) to give 7-ethyl-6-methoxy-N-(4-methylsulfonylphenyl)isoquinolin-1-amine 17 (0.18 g, 50.34% yield) as light yellow oil. ¹H NMR (400 MHz DMSO-d₆): δ=9.47 (s, 1H), 8.26 (s, 1H), 8.09 (d, J=8.8 Hz, 2H), 7.99 (d, J=5.6 Hz, 1H), 7.82 (d, J=8.8 Hz, 2H), 7.27 (s, 1H), 7.24 (d, J=5.8 Hz, 1H), 3.95 (s, 3H), 3.16 (s, 3H), 2.78 (q, J=7.4 Hz, 2H), 1.27 (t, J=7.4 Hz, 3H).

### 7-ethyl-1-(4-methylsulfonylanilino)isoquinolin-6-ol (18)

The above compound was prepared according to Scheme 28 and under the specific conditions below:

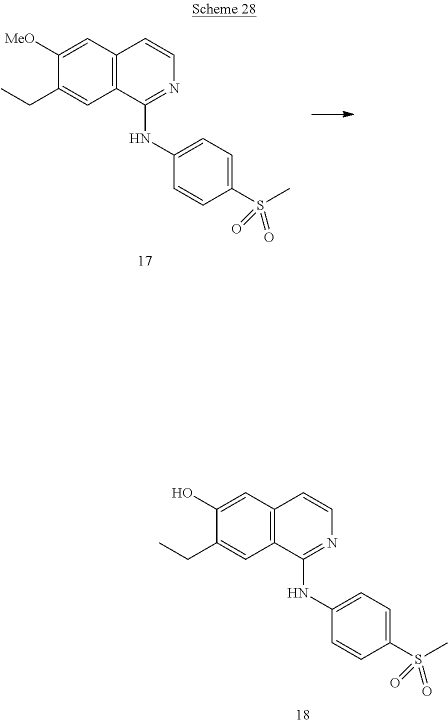

To a solution of 7-ethyl-6-methoxy-N-(4-methylsulfonylphenyl)isoquinolin-1-amine 17 (180 mg, 454.50 μmol) in DCM (2 mL) was added dropwise BBr₃ (569.31 mg, 2.27 mmol) dropwise, then the mixture was stirred at 20° C. for 4 hours. LC-MS showed starting material was consumed completely and one main peak with desired mass was detected. The mixture was adjusted to pH=9 with saturated NaHCO₃ aqueous solution, then the mixture extracted with ethyl acetate (2×100 mL). The combined organic phase was washed with water and brine (2×100 mL), dried over Na₂SO₄, filtered and concentrated under reduced pressure to give crude product. The crude product was purified by prep-HPLC (neutral condition), then lyophilized to give 7-ethyl-1-(4-methylsulfonylanilino)isoquinolin-6-ol (18) (0.023 g, 14.78% yield) as off white solid. ¹H NMR (400 MHz DMSO-d₆): δ=10.31 (br d, J=1.1 Hz, 1H), 9.41 (s, 1H), 8.22 (s, 1H), 8.08 (br d, J=8.5 Hz, 2H), 7.90 (br d, J=5.6 Hz, 1H), 7.81 (br d, J=8.4 Hz, 2H), 7.15-6.93 (m, 2H), 3.15 (s, 3H), 2.76 (q, J=7.3 Hz, 2H), 1.27 (br t, J=7.4 Hz, 3H).

### 7-bromo-1-(4-methylsulfonylanilino)isoquinolin-6-ol (19)

The above compound was prepared according to Scheme 29 and under the specific conditions below:

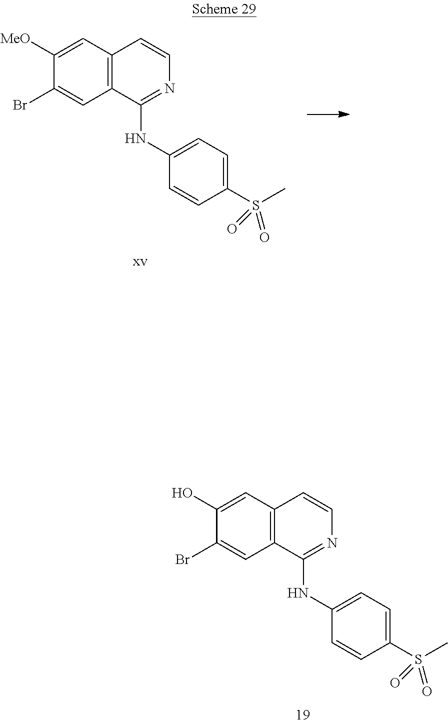 [SMILES: COc1cc2ccnc(Nc3ccc(S(C)(=O)=O)cc3)c2cc1Br ; CS(=O)(=O)c1ccc(Nc2nccc3cc(O)c(Br)cc23)cc1]

To a 10 mL stand-up bottle was added 7-bromo-6-methoxy-N-(4-methylsulfonylphenyl)isoquinolin-1-amine xv (0.2 g, 441.96 μmol) at 20° C., then BBr₃ (0.2 mL) was added to the mixture dropwise at 20° C. The mixture was stirred at 70° C. for 6 hours.

LC-MS showed 18% of starting material remained and 53% of desired compound was detected. The mixture was cooled to 20° C., and added to saturated NaHCO₃ aqueous solution (10 mL) dropwise at 0° C., then the mixture extracted with ethyl acetate (2×20 mL). The combined organic phase was washed with brine (2×20 mL), dried over Na₂SO₄, filtered and the filtrate was concentrated under reduced pressure. The crude product was purified by prep-HPLC neutral condition, then lyophilized to give 7-bromo-1-(4-methylsulfonylanilino)isoquinolin-6-ol (19) (4 mg, 2.01% yield) as off white solid. ¹H NMR (400 MHz DMSO-d₆): δ=11.20 (s, 1H) 9.56 (br s, 1H) 8.84 (s, 1H) 8.11 (br d, J=8.63 Hz, 2H) 7.98 (br d, J=5.75 Hz, 1H) 7.83 (br d, J=8.63 Hz, 2H) 7.23 (s, 1H) 7.16 (br d, J=5.75 Hz, 1H) 3.16 (s, 3H).

### N7-benzyl-6-methoxy-N1-(4-methylsulfonylphenyl)isoquinoline-1,7-diamine (20)

The above compound was prepared according to Scheme 30 and under the specific conditions below:

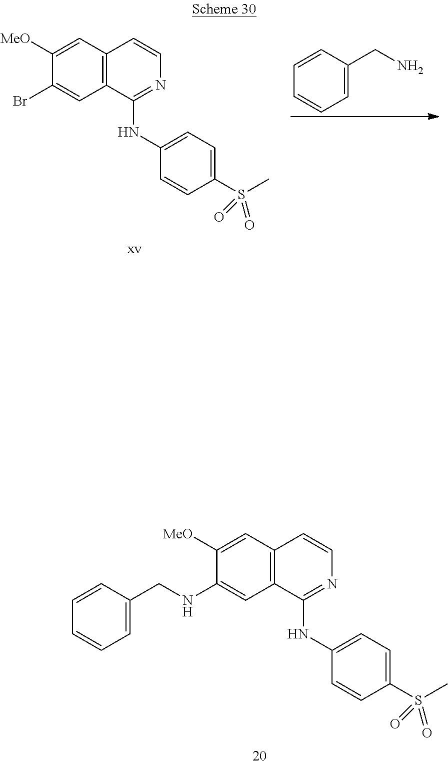 [SMILES: COc1cc2ccnc(Nc3ccc(S(C)(=O)=O)cc3)c2cc1Br ; NCc1ccccc1 ; COc1cc2ccnc(Nc3ccc(S(C)(=O)=O)cc3)c2cc1NCc1ccccc1]

To a solution of 7-bromo-6-methoxy-N-(4-methylsulfonylphenyl)isoquinolin-1-amine xv (50 mg, 122.77 μmol) in 1,4-dioxane (1 mL) was added phenylmethanamine (39.46 mg, 368.30 mol) and Cs₂CO₃ (120.00 mg, 368.30 μmol) at 25° C. The flask was filled with N₂ and evacuated (3×). Pd₂(dba)₃ (5.62 mg, 6.14 μmol) and Xantphos (7.10 mg, 12.28 μmol) were added to the mixture, and the flask was filled with N₂ and evacuated (3×). The mixture was stirred at 100° C. for 12 hrs. LCMS showed all the starting materials were consumed and the desired Ms was detected. The reaction mixture was filtered and the filtrate was concentrated under reduced pressure to give the crude product. The crude product was directly purified by Prep-HPLC and lyophilized to give N7-benzyl-6-methoxy-N1-(4-methylsulfonylphenyl)isoquinoline-1,7-diamine (20) (16.3 mg, 30.63% yield) as a light yellow solid. ¹H NMR (400 MHz DMSO-d₆): δ 9.11 (s, 1H), 8.04-7.94 (m, 2H), 7.83-7.75 (m, 3H), 7.44 (d, J=7.3 Hz, 2H), 7.30 (t, J=7.5 Hz, 2H), 7.26-7.20 (m, 2H), 7.19-7.13 (m, 2H), 6.06 (t, J=6.2 Hz, 1H), 4.55 (d, J=5.6 Hz, 2H), 3.98 (s, 3H), 3.14 (s, 3H).

### N7-(cyclopropylmethyl)-6-methoxy-N1-(4-methylsulfonylphenyl)isoquinoline-1,7-diamine (21)

The above compound was prepared according to Scheme 31 and under the specific conditions

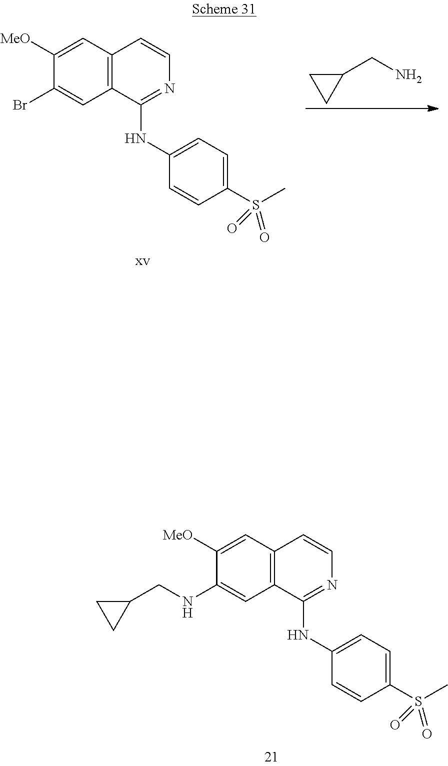 [SMILES: COc1cc2ccnc(Nc3ccc(S(C)(=O)=O)cc3)c2cc1Br ; NCC1CC1 ; COc1cc2ccnc(Nc3ccc(S(C)(=O)=O)cc3)c2cc1NCC1CC1]

To a solution of 7-bromo-6-methoxy-N-(4-methylsulfonylphenyl)isoquinolin-1-amine xv (50 mg, 122.77 μmol) in 1,4-dioxane (1 mL) was added cyclopropylmethanamine (26.19 mg, 368.30 μmol) and Cs₂CO₃ (120.00 mg, 368.30 μmol) at 25° C. The flask was filled with N₂ and evacuated (3×). Pd₂(dba)₃ (5.62 mg, 6.14 μmol,) and Xantphos (7.10 mg, 12.28 μmol) were added to the mixture, and the flask was filled with N₂ and evacuated (3×). The mixture was stirred at 100° C. for 12 hrs. LCMS showed all the starting materials were consumed and the desired Ms was detected. The reaction mixture was filtered and the filtrate was concentrated under reduced pressure to give the crude product. The crude product was directly purified by Prep-HPLC and lyophilized to give N7-(cyclopropylmethyl)-6-methoxy-N1-(4-methylsulfonylphenyl)isoquinoline-1,7-diamine (21) (10.7 mg, 21.93% yield) as a light yellow solid. ¹H NMR (400 MHz DMSO-d₆): δ 9.16 (s, 1H), 8.06-7.98 (m, 2H), 7.84-7.76 (m, 3H), 7.22 (s, 1H), 7.19-7.14 (m, 2H), 5.36 (t, J=5.6 Hz, 1H), 3.98 (s, 3H), 3.18-3.15 (m, 2H), 3.14 (s, 3H), 1.29-1.15 (m, 1H), 0.58-0.45 (m, 2H), 0.30 (q, J=4.8 Hz, 2H).

### 6-methoxy-N1-(4-methylsulfonylphenyl)-N7-propyl-isoquinoline-1,7-diamine (22)

The above compound was prepared according to Scheme 32 and under the specific conditions below:

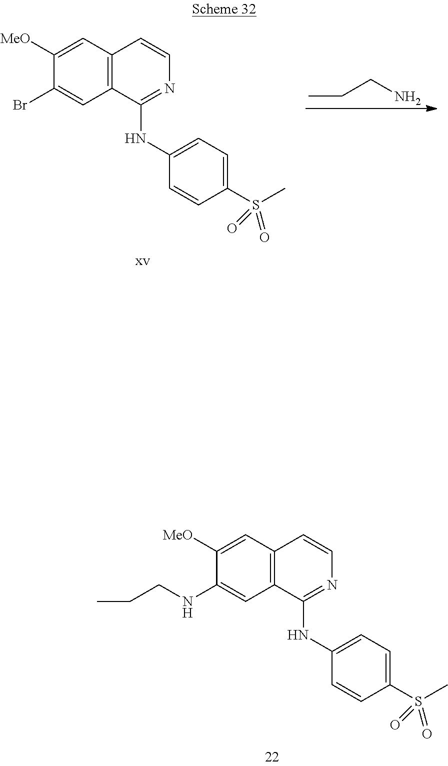

To a solution of 7-bromo-6-methoxy-N-(4-methylsulfonylphenyl)isoquinolin-1-amine xv Cs₂CO₃ (120.00 mg, 368.30 μmol) at 25° C. The flask was filled with N₂ and evacuated (3×). Pd₂(dba)₃ (5.62 mg, 6.14 μmol) and Xantphos (7.10 mg, 12.28 μmol) were added to the mixture, and the flask was filled with N₂ and evacuated (3×) The mixture was stirred at 100° C. for 12 hrs. LCMS showed all the starting materials were consumed and the desired Ms was detected. The reaction mixture was filtered and the filtrate was concentrated under reduced pressure to give the crude product. The crude product was directly purified by Prep-HPLC and lyophilized to give 6-methoxy-N1-(4-methylsulfonylphenyl)-N7-propyl-isoquinoline-1,7-diamine (22) (13.4 mg, 26.79% yield) as a light-yellow solid. ¹H NMR (400 MHz DMSO-d₆): δ 9.18 (s, 1H), 8.06-7.95 (m, 2H), 7.86-7.73 (m, 3H), 7.21-7.11 (m, 3H), 5.37 (br t, J=5.6 Hz, 1H), 3.96 (s, 3H), 3.28-3.21 (m, 2H), 3.14 (s, 3H), 1.76-1.64 (m, 2H), 0.99 (t, J=7.4 Hz, 3H).

### Intermediate 6-bromo-7-methoxy-N-(4-methylsulfonylphenyl)isoquinolin-1-amine xix

### Step 1:

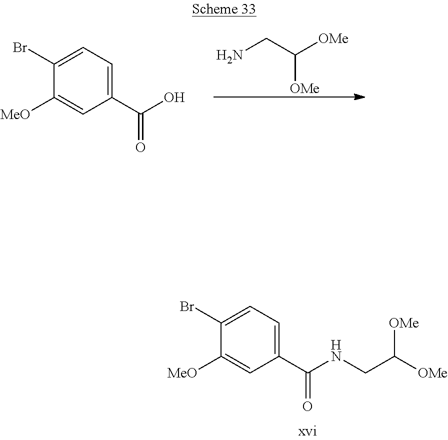 [SMILES: COc1cc(C(=O)O)ccc1Br ; COC(CN)OC ; COc1cc(C(=O)NCC(OC)OC)ccc1Br]

To a mixture of 4-bromo-3-methoxy-benzoic acid (10.0 g, 43.28 mmol) in dichloromethane (200 mL) was added 2-(7-azabenzotriazol-1-yl)-N,N,N′,N′-tetramethyluronium hexafluorophosphate (18.10 g, 47.61 mmol) and triethylamine (5.26 g, 51.94 mmol) in one portion at 20° C. under N₂. The mixture was stirred at 20° C. for 30 minutes, then 2,2-dimethoxyethanamine (5.23 g, 49.77 mmol) was added to the reaction mixture at 0° C. The reaction mixture was stirred at 20° C. for 2.5 hours. LCMS showed the reaction was completed. The mixture was poured into ice-water (100 mL) and stirred for 5 minutes. The aqueous phase was extracted with ethyl acetate (200 mL×3). The combined organic phase was washed with brine (100 mL), dried with anhydrous Na₂SO₄, filtered and concentrated in vacuum. The residue was purified by silica gel chromatography (eluted with ethyl acetate in petroleum ether from 0% to 20%) to give 4-bromo-N-(2,2-dimethoxyethyl)-3-methoxy-benzamid xvi (12 g, 87.14% yield) as a light-yellow solid.

### Step2:

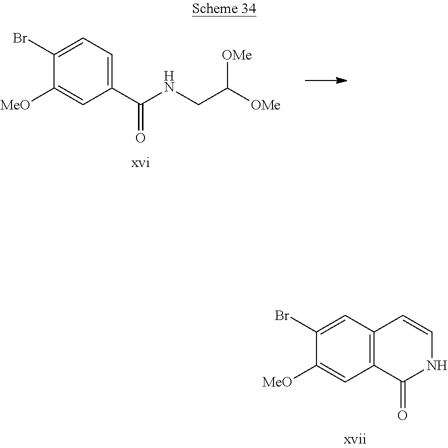 [SMILES: COc1cc(C(=O)NCC(OC)OC)ccc1Br ; COc1cc2c(=O)[nH]ccc2cc1Br]

To a mixture of 4-bromo-N-(2,2-dimethoxyethyl)-3-methoxy-benzamide xvi (12 g, 37.72 mmol) in sulfuric acid (3.70 g, 37.72 mmol) in one portion at 20° C. under N₂. The reaction mixture was stirred at 20° C. for 2 hours. Then the reaction mixture was stirred at 60° C. for 2 hours. LCMS showed all the starting materials were consumed and the desired Ms was detected. The reaction mixture was quenched with ice water (100 mL), filtered and the filter cake was washed with water (3×100 mL) and concentrated under reduced pressure to give the crude 6-bromo-7-methoxy-2H-isoquinonil-1-one xvii (9 g, 89.22% yield) as a yellow solid.

### Step 3:

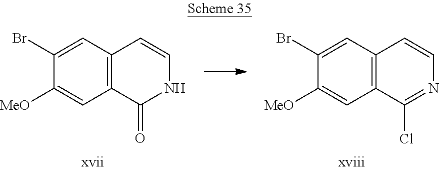 [SMILES: COc1cc2c(=O)[nH]ccc2cc1Br ; COc1cc2c(Cl)nccc2cc1Br]

To a mixture of 6-bromo-7-methoxy-2H-isoquinonil-1-one xvii (9.00 g, 33.65 mmol) in toluene (20 mL) was added phosphorus oxychloride (40 mL) to the mixture at 20° C. The mixture was stirred at 110° C. for 6 hrs. LCMS showed all the starting materials were consumed and the desired Ms was detected. The reaction mixture was concentrated under reduced pressure to give the residue. The residue was triturated in ethyl acetate (20 mL) at 25° C. for 1 hr and filtered. The filter cake was concentrated under reduced pressure to give 6-bromo-1-chloro-7-methoxy-isoquinolin xviii (5.4 g, 55.94% yield) as a white solid.

### Step 4:

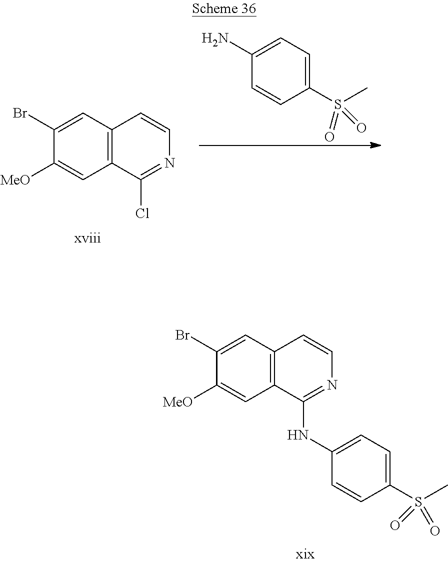 [SMILES: COc1cc2c(Cl)nccc2cc1Br ; CS(=O)(=O)c1ccc(N)cc1 ; COc1cc2c(Nc3ccc(S(C)(=O)=O)cc3)nccc2cc1Br]

To a mixture of 6-bromo-1-chloro-7-methoxy-isoquinolin xviii (2 g, 6.97 mmol) in isopropanol (40 mL) was added 4-methylsulfonylaniline (1.19 g, 6.97 mmol) and HCl/dioxane (6 M, 1.74 mL) at 20° C. Then the mixture was stirred at 90° C. for 16 hrs. LCMS showed all the starting materials were consumed and the desired Ms was detected. The reaction mixture was filtered, the filter cake was washed with isopropanol (10 mL), then suspended in ethyl acetate (30 mL) and adjusted to pH 8 with saturated aqueous NaHCO₃ at 0° C. The mixture was filtered and the filter cake was washed with ethyl acetate (10 mL) and water (10 mL) and concentrated under reduced pressure to give 6-bromo-7-methoxy-N-(4-methylsulfonylphenyl)isoquinolin-1-amine xix (1.4 g, 46.84% yield) as a white solid. ¹H NMR (400 MHz DMSO-d₆): δ 9.56 (s, 1H), 8.25 (s, 1H), 8.07 (d, J=8.8 Hz, 2H), 8.01 (d, J=5.6 Hz, 1H), 7.94 (s, 1H), 7.86 (d, J=8.9 Hz, 2H), 7.27 (d, J=5.8 Hz, 1H), 4.07 (s, 3H), 3.17 (s, 3H).

### N6-(cyclopropylmethyl)-7-methoxy-N1-(4-methylsulfonylphenyl)isoquinoline-1,6-diamine (23)

The above compound was prepared according to Scheme 37 and under the specific conditions below:

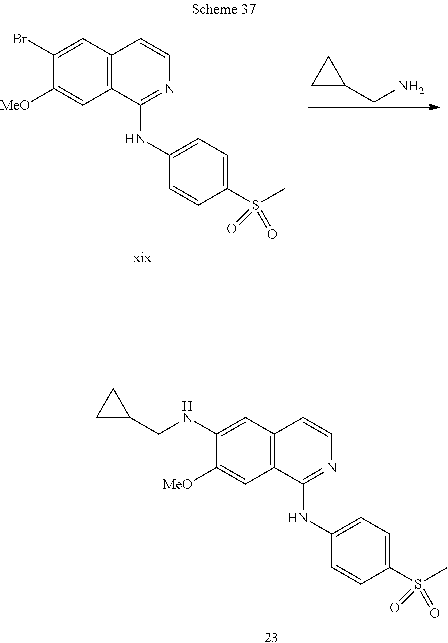 [SMILES: COc1cc2c(Nc3ccc(S(C)(=O)=O)cc3)nccc2cc1Br ; NCC1CC1 ; "xix" ; COc1cc2c(Nc3ccc(S(C)(=O)=O)cc3)nccc2cc1NCC1CC1 ; "23"]

To a solution of 6-bromo-7-methoxy-N-(4-methylsulfonylphenyl)isoquinolin-1-amine xix (50 mg, 110.49 μmol) in 1,4-dioxane (1 mL) was added cyclopropylmethanamine (23.57 mg, 331.47 μmol) and Cs₂CO₃ (108.00 mg, 331.47 μmol) at 25° C. The flask was filled with N₂ and evacuated (3×). Pd₂(dba)₃ (5.06 mg, 5.52 μmol) and Xantphos (6.39 mg, 11.05 μmol) were added to the mixture, and the flask was filled with N₂ and evacuated (3×). The mixture was stirred at 100° C. for 12 hrs. LCMS showed all the starting materials were consumed and the desired Ms was detected. The reaction mixture was filtered and the filtrate was concentrated under reduced pressure to give the crude product. The crude product was directly purified by Prep-HPLC and lyophilized to give N6-(cyclopropylmethyl)-7-methoxy-N1-(4-methylsulfonylphenyl)isoquinoline-1,6-diamine (23) (10 mg, 23, 20.83% yield) as a light yellow solid. ¹H NMR (400 MHz DMSO-d₆): δ 9.29-9.15 (m, 1H), 8.02 (br d, J=8.0 Hz, 2H), 7.89-7.75 (m, 3H), 7.67-7.59 (m, 1H), 7.07 (br d, J=4.8 Hz, 1H), 6.73 (s, 1H), 5.74 (br d, J=4.5 Hz, 1H), 4.02 (s, 3H), 3.14 (s, 3H), 3.09 (br d, J=5.4 Hz, 2H), 1.26-1.13 (m, 1H), 0.50 (br d, J=7.8 Hz, 2H), 0.28 (br d, J=3.6 Hz, 2H).

### 6-(azetidin-1-yl)-7-methoxy-N-(4-methylsulfonylphenyl)isoquinolin-1-amine (24)

The above compound was prepared according to Scheme 38 and under the specific conditions below:

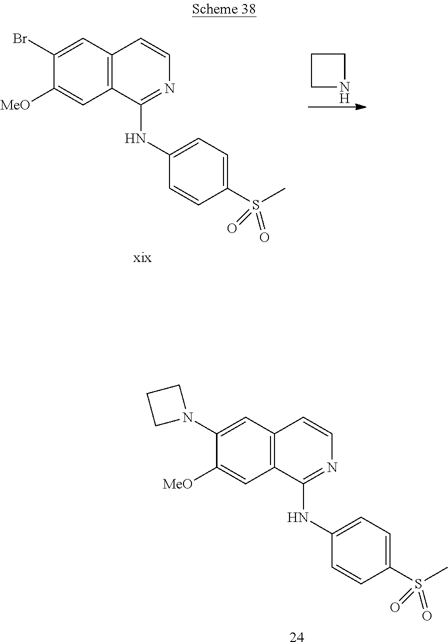

To a solution of 6-bromo-7-methoxy-N-(4-methylsulfonylphenyl)isoquinolin-1-amine xix (50 mg, 110.49 μmol) in 1,4-dioxane (1 mL) was added azetidine (18.92 mg, 331.47 μmol) and Cs₂CO₃ (108.00 mg, 331.47 μmol) at 25° C. The flask was filled with N₂ and evacuated (3×). Pd₂(dba)₃ (5.06 mg, 5.52 μmol) and Xantphos (6.39 mg, 11.05 μmol) were added to the mixture, and the flask was filled with N₂ and evacuated (3×) The mixture was stirred at 100° C. for 12 hrs. LCMS showed all the starting materials were consumed and the desired Ms detected. The reaction mixture was filtered and the filtrate was concentrated under reduced pressure to give the crude product. The crude product was directly purified by Prep-HPLC and lyophilized to give 6-(azetidin-1-yl)-7-methoxy-N-(4-methylsulfonylphenyl)isoquinolin-1-amine (24) (9.3 mg, 17.36% yield) as a light yellow solid. ¹H NMR (400 MHz DMSO-d₆): δ 9.25 (s, 1H), 8.06-7.99 (m, 2H), 7.87-7.77 (m, 3H), 7.62 (s, 1H), 7.08 (d, J=5.9 Hz, 1H), 6.55 (s, 1H), 4.09-4.00 (m, 4H), 3.93 (s, 3H), 3.15 (s, 3H), 2.31-2.24 (m, 2H), 2.07 (s, 1H).

### 6-ethyl-1-[(4-methanesulfonylphenyl)amino]isoquinolin-7-ol (25)

The above compound was prepared according to Schemes 39 to 41 and under the specific conditions below:

### Step 1:

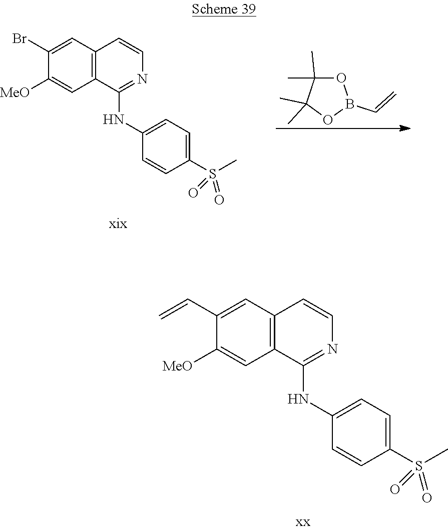

To a mixture of 6-bromo-7-methoxy-N-(4-methylsulfonylphenyl)isoquinolin-1-amine xix (700 mg, 1.63 mmol) in tetrahydrofuran (12 mL) and water (3 mL) was added 4,4,5,5-tetramethyl-2-vinyl-1,3,2-dioxaborolane (276.62 mg, 1.80 mmol) and K₃PO₄ (693.18 mg, 3.27 mmol) at 20° C. The flask was filled with N₂ and evacuated (3×). Ditert-butyl(cyclopentyl)phosphane; dichloropalladium; iron (106.42 mg, 163.28 μmol) was added to the mixture, and the flask was filled with N₂ and evacuated (3×). Then the mixture was stirred at 80° C. for 4 hrs. LCMS showed all the starting materials were consumed and the desired Ms was detected. The reaction mixture was concentrated under reduced pressure, and extracted with dichloromethane (3×30 mL). The combined organic phases were washed with brine (50 mL), dried over anhydrous sodium sulfate, filtered and concentrated under reduced pressure to give the crude product. The crude product was purified by prep-HPLC and lyophilized to give 6-vinyl-7-methoxy-N-(4-methylsulfonylphenyl)isoquinolin-1-amine xx (380 mg, 59.10% yield) as a yellow solid.

### Step 2:

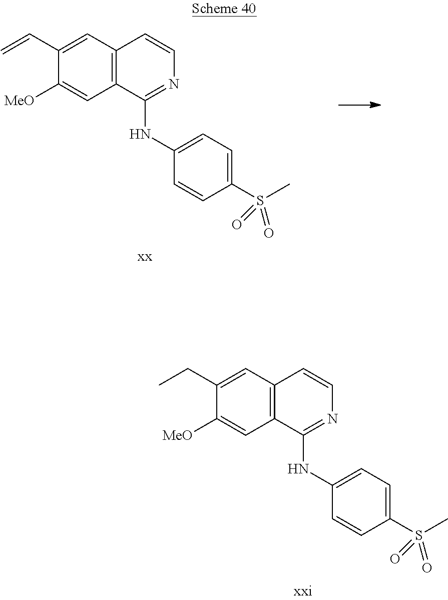

To a mixture of 6-vinyl-7-methoxy-N-(4-methylsulfonylphenyl)isoquinolin-1-amine xx (380 mg, 964.95 μmol) in methyl alcohol (10 mL) was added Pd/C (102.69 mg, 964.95 μmol) at 20° C. Then the mixture was stirred at 20° C. for 2 hrs. LCMS showed all the starting materials were consumed and the desired Ms was detected. The reaction mixture was filtered through a pad of celite and the filtrate was concentrated under reduced pressure to give 6-ethyl-7-methoxy-N-(4-methylsulfonylphenyl)isoquinolin-1-amine xxi (220 mg, 60.77% yield) as a white solid

### Step 3:

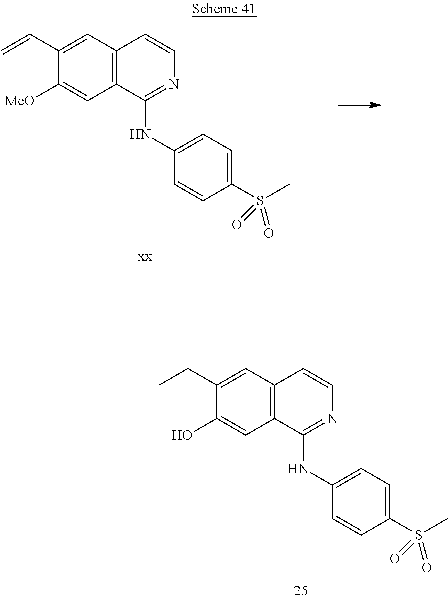

To a solution of 6-ethyl-7-methoxy-N-(4-methylsulfonylphenyl)isoquinolin-1-amine xxi (200 mg, 505.00 μmol) in acetic acid (4 mL) was added hydrobromic acid (185.72 mg, 757.50 μmol). The reaction solution was allowed to warm to 100° C. slowly and stirred for 12 hrs. LCMS showed all the starting materials were consumed, desired Ms was detected. The reaction mixture was concentrated under reduced pressure and adjusted to pH=8 with aq.NaHCO₃. The mixture was filtered and the filter cake was concentrated under reduced pressure to give the crude product. The crude product was purified by prep-HPLC and lyophilized to give 6-ethyl-7-hydroxy-N-(4-methylsulfonylphenyl)isoquinolin-1-amine (25) (62.4 mg, 35.69% yield) as a light yellow solid. ¹H NMR (400 MHz DMSO-d₆): δ 10.09-9.97 (m, 1H), 9.50-9.42 (m, 1H), 7.99-7.92 (m, 2H), 7.90 (d, J=5.7 Hz, 1H), 7.79 (d, J=8.8 Hz, 2H), 7.62 (s, 2H), 7.24 (d, J=5.7 Hz, 1H), 3.15 (s, 3H), 2.74 (q, J=7.5 Hz, 2H), 1.24 (t, J=7.5 Hz, 3H).

### N-[4-(ethanesulfonyl)phenyl]-6,7-diethoxyisoquinolin-1-amine (26)

The above compound was prepared according to Scheme 42 under the specific conditions below:

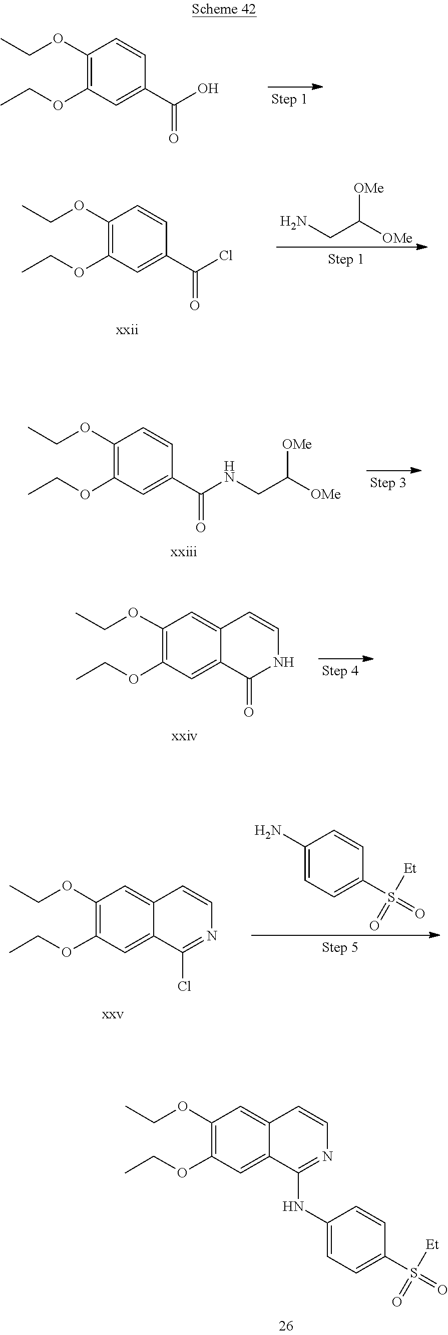

Step 1: To a solution of 3,4-diethoxybenzoic acid (25 g, 118.92 mmol) in dichloromethane (250 mL) was added dimethyl formamide (434.59 mg, 5.95 mmol) at 20° C. Then, the mixture was added oxalyl dichloride (18.11 g, 142.70 mmol) drop-wise at 0° C. under N₂. The mixture was stirred at 20° C. for 2 hrs. TLC showed the start material was consumed and one new spot with small polarity formed. The mixture was concentrated under reduced pressure to give xxii (28 g, 92.67% yield) as a yellow solid. ¹H NMR (400 MHz, DMSO-d₆): δ 7.52 (dd, J=8.38, 1.88 Hz, 1H) 7.41 (d, J=2.00 Hz, 1H) 7.01 (d, J=8.50 Hz, 1H) 4.06 (dq, J=16.63, 7.00 Hz, 4H) 1.33 (td, J=6.94, 3.50 Hz, 6H).

Step 2: To a solution of 2,2-dimethoxyethanamine (11.74 g, 111.63 mmol) in tetrahydrofuran (212 mL) was added N,N-diisopropylethylamine (21.64 g, 167.45 mmol) at 20° C. Then, the mixture was added a solution of xxii (31.2 g, 122.80 mmol) in tetrahydrofuran (100 mL) drop-wise at 0° C. under N₂. The mixture was stirred at 20° C. for 16 hrs. LCMS showed the starting material was consumed and the product was detected. The mixture was quenched by the ice water (600 mL) and filtered. The filter cake was concentrated under reduced pressure to give xxiii (31.3 g, 84.87% yield) as a white solid. ¹H NMR (400 MHz, DMSO-d6): δ 8.41 (br t, J=5.82 Hz, 1H) 7.38-7.56 (m, 2H) 6.99 (d, J=8.25 Hz, 1H) 4.49 (t, J=5.50 Hz, 1H) 4.06 (qd, J=6.90, 4.69 Hz, 4H) 3.32-3.36 (m, 2H) 3.29 (s, 6H) 1.34 (td, J=6.94, 1.88 Hz, 6H).

Step 3: A solution of 2 (15.9 g, 48.13 mmol) in H₂SO₄ (6.41 mL) at 20° C. The mixture was stirred at 80° C. for 16 hrs. LCMS showed the starting material was consumed and the product was detected. One additional vial in 5 g scale was set up as described above. The mixtures were quenched by the ice water (30 mL) and adjusted to pH 7 with 0.5 N of NaOH (300 mL). Then the mixture was filtered, the filter cake was concentrated under reduced pressure to give crude product. The crude product was stirred in ethyl acetate (100 mL) to form a slurry, filtered to give xxiv (8.4 g, 38.84% yield) as a grey solid. ¹H NMR (400 MHz, DMSO-d₆): δ 11.04 (br s, 1H) 7.52 (s, 1H) 7.13 (s, 1H) 7.03 (br t, J=6.19 Hz, 1H) 6.44 (d, J=7.00 Hz, 1H) 3.97-4.35 (m, 4H) 1.30-1.48 (m, 6H).

Step 4: A solution of xxiv (6.9 g, 20.11 mmol) in POCl₃ (41.4 mL) at 20° C. was heated and stirred at 110° C. for 1 hr. LCMS showed the starting material was consumed and the product was detected. The reaction mixture was concentrated under reduced pressure to remove POCl₃. The residue was diluted with water (80 mL) and adjusted to pH 7 with saturated aqueous NaHCO₃ (90 mL). Then the mixture was filtered, the filter cake was concentrated under reduced pressure to give 1-chloro-6,7-diethoxyisoquinoline xxv (6.8 g, 94.25% yield) as a grey solid. ¹H NMR (400 MHz, DMSO-d₆): δ 8.09 (d, J=5.50 Hz, 1H) 7.68 (d, J=5.50 Hz, 1H) 7.43 (d, J=15.76 Hz, 2H) 4.22 (q, J=6.88 Hz, 4H) 1.43 (td, J=6.94, 1.13 Hz, 6H).

Step 5: To a mixture of 1-chloro-6,7-diethoxyisoquinoline xxv (150 mg, 595.93 μmol) and 4-ethylsulfonylaniline (110.39 mg, 595.93 μmol) in dioxane (3 mL) was added Cs₂CO₃ (388.33 mg, 1.19 mmol) at 20° C., the vessel was evacuated and backfilled with nitrogen (this process was repeated three times), then (1E,4E)-1,5-diphenylpenta-1,4-dien-3-one; palladium (32.74 mg, 35.76 μmol) and dicyclohexyl-[2-(2,6-dimethoxyphenyl)phenyl]phosphane (24.46 mg, 59.59 μmol) was added to the mixture under nitrogen, the vessel was evacuated and backfilled with nitrogen (this process was repeated three times), the reaction mixture was heated to 100° C. and stirred for 12 hrs. LCMS showed the starting material was consumed and the desired product was detected. The reaction mixture was filtered and the filtrate was concentrated under reduced pressure to give crude product. The crude product was purified by prep-HPLC and lyophilized to give N-[4-(ethanesulfonyl)phenyl]-6,7-diethoxyisoquinolin-1-amine (26) (138.3 mg, 57.02% yield, 98.4% purity) as a yellow solid. ¹H NMR (400 MHz, DMSO-d₆): δ=9.34 (s, 1H) 8.05 (d, J=8.88 Hz, 2H) 7.94 (d, J=5.63 Hz, 1H) 7.73-7.82 (m, 3H) 7.29 (s, 1H) 7.22 (d, J=5.63 Hz, 1H) 4.22 (dq, J=18.39, 6.96 Hz, 4H) 3.22 (q, J=7.30 Hz, 2H) 1.35-1.50 (m, 6H) 1.12 (t, J=7.38 Hz, 3H)

### 6,7-diethoxy-N-[4-(oxetan-3-yl)phenyl]isoquinolin-1-amine (27)

The above compound was prepared according to Scheme 43 under the specific conditions below:

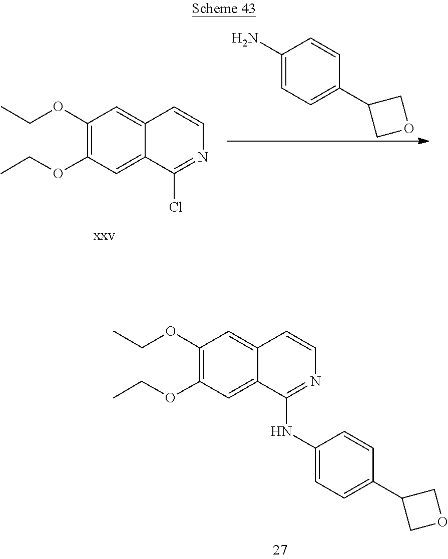 [SMILES: CCOc1cc2ccnc(Cl)c2cc1OCC ; Nc1ccc(C2COC2)cc1 ; CCOc1cc2ccnc(Nc3ccc(C4COC4)cc3)c2cc1OCC]

A mixture of xxv (150 mg, 595.93 μmol), 4-(oxetan-3-yl)aniline (97.80 mg, 655.52 μmol) and Cs₂CO₃ (388.33 mg, 1.19 mmol) in dioxane (3 mL) was degassed and purged with N₂ for 3 times, and then SPhos (24.46 mg, 59.59 μmol) and Pd₂(dba)₃ (27.29 mg, 29.80 μmol) was added under N₂. The mixture was stirred at 100° C. for 16 hours under N₂ atmosphere. LCMS showed the reaction was completed. The mixture was filtered through celite, the filtrate was concentrated under reduced pressure to give a residue. The residue was stirred in a mixture of petroleum ether and ethyl acetate (20 mL, 9:1) for 15 minutes, the solid was collected and then purified by prep-TLC (SiO₂, petroleum ether:tetrahydrofuran=1:1) to give crude product. The crude product was stirred in a mixture of petroleum ether and ethyl acetate (15 mL, 4:1) for 15 minutes, the solid was collected to give 6,7-diethoxy-N-[4-(oxetan-3-yl)phenyl]isoquinolin-1-amine (27) as a white solid (111.5 mg, 50.98% yield). ¹H NMR (400 MHz, DMSO-d₆): δ=8.84 (s, 1H), 7.85-7.74 (m, 4H), 7.34 (d, J=8.4 Hz, 2H), 7.22 (s, 1H), 7.03 (d, J=5.8 Hz, 1H), 4.94 (dd, J=5.8, 8.3 Hz, 2H), 4.63 (t, J=6.3 Hz, 2H), 4.27-4.13 (m, 5H), 1.42 (q, J=7.3 Hz, 6H).

### 6,7-diethoxy-N-[4-(1,2-oxazol-3-yl)phenyl]isoquinolin-1-amine (28)

The above compound was prepared according to Scheme 44 under the specific conditions below:

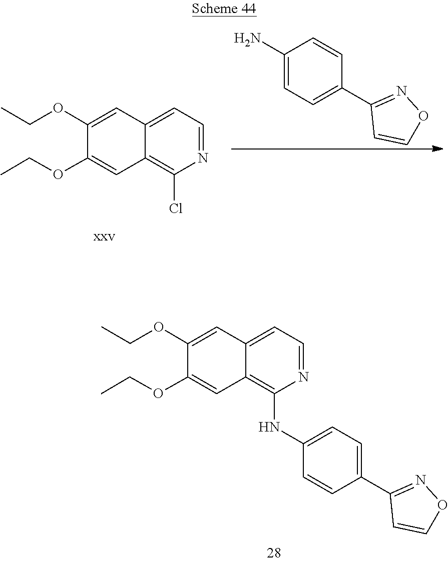 [SMILES: CCOc1cc2ccnc(Cl)c2cc1OCC ; Nc1ccc(-c2ccon2)cc1 ; CCOc1cc2ccnc(Nc3ccc(-c4ccon4)cc3)c2cc1OCC]

To a solution of xxv (150 mg, 595.93 μmol, 1 eq) in dioxane (3 mL) was added 4-(1,2-Oxazol-3-yl)aniline (105.00 mg, 655.52 μmol, 1.1 eq) and Cs₂CO₃ (388.33 mg, 1.19 mmol, 2 eq) at 25° C. The mixture degassed and purged with N₂ for 3 times. SPhos (24.46 mg, 59.59 μmol, 0.1 eq) Pd₂(dba)₃ (27.29 mg, 29.80 μmol, 0.05 eq) was added into the reaction mixture at 25° C. The mixture was degassed and purged with N₂ for 3 times and stirred at 100° C. for 12 hrs. LC-MS showed starting material was consumed completely and desired Ms was detected. The reaction mixture was filtered and filtrate was concentrated under reduced pressure to give a residue. The crude product was purified by pre-HPLC to give 6,7-diethoxy-N-[4-(1,2-oxazol-3-yl)phenyl]isoquinolin-1-amine (28) as a yellow solid (114.6 mg, 305.26 μmol, 51.22% yield). ¹H NMR (400 MHz, DMSO-d₆): δ ppm 9.07 (s, 1H), 8.95 (d, J=1.63 Hz, 1H), 7.97 (d, J=8.76 Hz, 2H), 7.90 (d, J=5.63 Hz, 1H), 7.77-7.86 (m, 3H), 7.26 (s, 1H), 7.02-7.16 (m, 2H), 4.05-4.36 (m, 4H), 1.43 (dt, J=9.63, 7.00 Hz, 6H)

### 6,7-diethoxy-N-[4-(1,2-oxazol-5-yl)phenyl]isoquinolin-1-amine (29)

The above compound was prepared according to Scheme 45 under the specific conditions

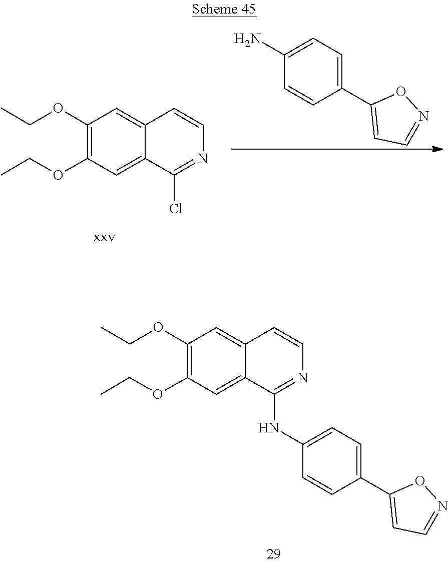 [SMILES: CCOc1cc2ccnc(Cl)c2cc1OCC ; Nc1ccc(-c2ccno2)cc1 ; CCOc1cc2ccnc(Nc3ccc(-c4ccno4)cc3)c2cc1OCC]

To a solution of xxv (150 mg, 595.93 μmol, 1 eq) in dioxane (3 mL) was added 4-isoxazol-5-ylaniline (105.00 mg, 655.52 μmol, 1.1 eq) and Cs₂CO₃ (388.33 mg, 1.19 mmol, 2 eq) at 25° C. The mixture degassed and purged with N₂ for 3 times. SPhos (24.46 mg, 59.59 μmol, 0.1 eq) Pd₂(dba)₃ (27.29 mg, 29.80 μmol, 0.05 eq) was added into the reaction mixture at 25° C. The mixture was degassed and purged with N₂ for 3 times and stirred at 60° C. for 12 hrs. LC-MS showed Reactant 1 was consumed completely and desired Ms was detected. The reaction mixture was filtered and concentrated under reduced pressure to give a residue. The residue was purified by pre-TLC (Petroleum ether/Ethyl acetate=1/1). The crude product was purified by pre-HPLC to give 6,7-diethoxy-N-[4-(1,2-oxazol-5-yl)phenyl]isoquinolin-1-amine (29) as a light yellow solid (27.4 mg, 72.40 μmol, 12.15% yield, 99.2% purity). ¹H NMR (400 MHz, DMSO-d₆): δ ppm 9.13 (s, 1H), 8.59 (d, J=1.75 Hz, 1H), 8.00 (d, J=8.88 Hz, 2H), 7.91 (d, J=5.63 Hz, 1H), 7.72-7.85 (m, 3H), 7.27 (s, 1H), 7.14 (d, J=5.63 Hz, 1H), 6.86 (d, J=1.75 Hz, 1H), 4.04-4.35 (m, 4H), 1.43 (dt, J=9.47, 7.02 Hz, 6H).

### 6,7-diethoxy-N-(3-fluoro-4-methanesulfonylphenyl)isoquinolin-1-amine (30)

The above compound was prepared according to Scheme 46 under the specific conditions below:

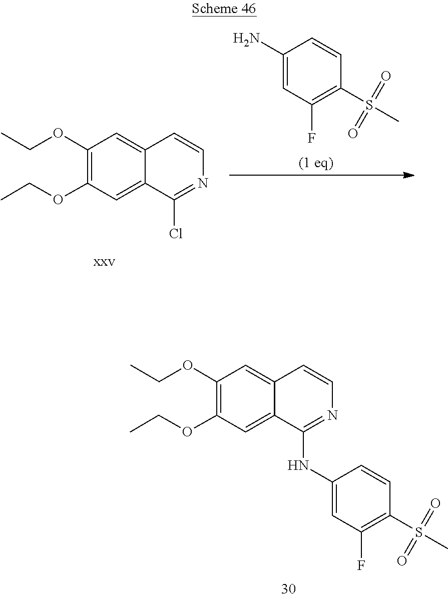 [SMILES: CCOc1cc2ccnc(Cl)c2cc1OCC ; CS(=O)(=O)c1ccc(N)cc1F ; CCOc1cc2ccnc(Nc3ccc(S(C)(=O)=O)c(F)c3)c2cc1OCC]

To a mixture of xxv (150 mg, 595.93 μmol) and 3-fluoro-4-methylsulfonyl-aniline (112.75 mg, 595.93 μmol) in dioxane (3 mL) was added Cs₂CO₃ (388.33 mg, 1.19 mmol) at 20° C., the vessel was evacuated and backfilled with nitrogen (this process was repeated three times), then (1E,4E)-1,5-diphenylpenta-1,4-dien-3-one; palladium (32.74 mg, 35.76 μmol) and dicyclohexyl-[2-(2,6-dimethoxyphenyl)phenyl]phosphane (24.46 mg, 59.59 μmol) was added to the mixture under nitrogen, the vessel was evacuated and backfilled with nitrogen (this process was repeated three times), the reaction mixture was heated to 100° C. and stirred for 12 hrs. LCMS showed the starting material was consumed and the desired product was detected. The reaction mixture was filtered and the filtrate was concentrated under reduced pressure to give crude product. The crude product was purified by prep-HPLC and lyophilized to give 6,7-diethoxy-N-(3-fluoro-4-methanesulfonylphenyl)isoquinolin-1-amine (30) as a yellow solid (163.5 mg, 67.16% yield, 99% purity). ¹H NMR (400 MHz, DMSO-d₆): δ=9.52 (s, 1H) 8.12-8.21 (m, 1H) 7.99 (d, J=5.63 Hz, 1H) 7.69-7.81 (m, 3H) 7.32 (s, 1H) 7.27 (d, J=5.63 Hz, 1H) 4.22 (dq, J=17.39, 7.00 Hz, 4H) 3.26 (s, 3H) 1.43 (dt, J=9.79, 6.99 Hz, 6H).

### 6,7-diethoxy-N-(2-fluoro-4-methanesulfonylphenyl)isoquinolin-1-amine (31)

The above compound was prepared according to Scheme 47 under the specific conditions below:

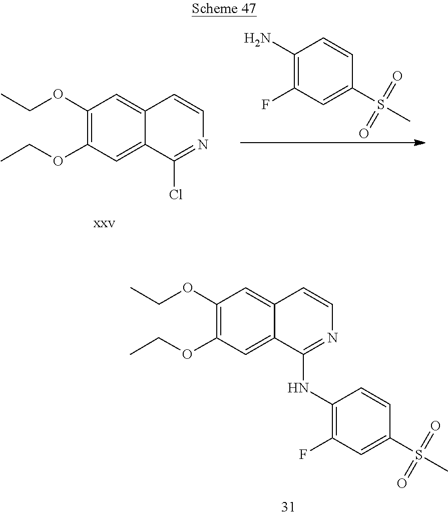 [SMILES: CCOc1cc2ccnc(Cl)c2cc1OCC ; CS(=O)(=O)c1ccc(N)c(F)c1 ; CCOc1cc2ccnc(Nc3ccc(S(C)(=O)=O)cc3F)c2cc1OCC]

To a solution of xxv (150 mg, 476.74 μmol) and 2-fluoro-4-methylsulfonyl-aniline (90.20 mg, 476.74 μmol) in dioxane (3 mL) was added Cs₂CO₃ (310.66 mg, 953.49 μmol) at 20° C. Then the mixture was added pd₂(dba)₃ (21.83 mg, 23.84 μmol) and sphos (19.57 mg, 47.67 μmol) at 20° C. under N₂. The mixture was stirred at 100° C. for 16 hrs. LCMS showed the starting material was consumed and the product was detected. The mixture was filtered through a celite pad, and the filtrate was concentrated under reduced pressure to give crude product. The crude product was purified by Prep-HPLC and lyophilized to give 6,7-diethoxy-N-(2-fluoro-4-methanesulfonylphenyl)isoquinolin-1-amine (31) as a white solid (137.7 mg, 70.34% yield). ¹H NMR (400 MHz, DMSO-d₆): δ 9.09 (s, 1H) 7.89 (t, J=8.07 Hz, 1H) 7.84 (d, J=5.63 Hz, 1H) 7.78 (dd, J=10.51, 2.00 Hz, 1H) 7.68-7.74 (m, 2H) 7.28 (s, 1H) 7.20 (d, J=5.75 Hz, 1H) 4.14-4.28 (m, 4H) 3.26 (s, 3H) 1.37-1.48 (m, 6H).

### N-[4-(cyclopropanesulfonyl)phenyl]-6,7-diethoxyisoquinolin-1-amine (32)

The above compound was prepared according to Scheme 48 under the specific conditions below:

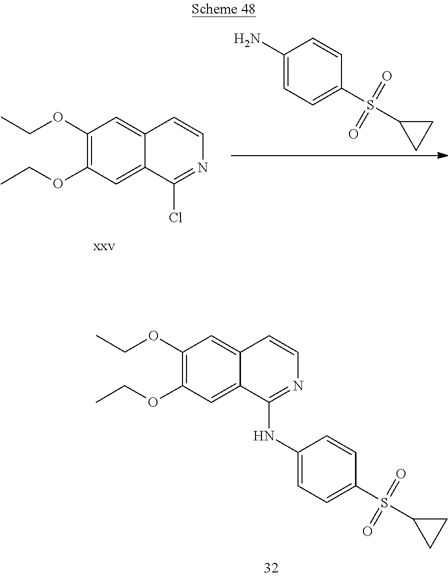 [SMILES: CCOc1cc2ccnc(Cl)c2cc1OCC ; Nc1ccc(S(=O)(=O)C2CC2)cc1 ; CCOc1cc2ccnc(Nc3ccc(S(=O)(=O)C4CC4)cc3)c2cc1OCC]

To a solution of xxv (130 mg, 413.18 μmol) and 4-cyclopropylsulfonylaniline (81.50 mg, 413.18 μmol) in dioxane (2.6 mL) was added Cs₂CO₃ (269.24 mg, 826.35 μmol) at 20° C. Then the mixture was added sphos (16.96 mg, 41.32 μmol) and pd₂(dba)₃ (18.92 mg, 20.66 μmol) at 20° C. under N₂. The mixture was stirred at 100° C. for 16 hrs. LCMS showed the starting material was consumed and the product was detected. The mixture was filtered through a celite pad, and the filtrate was concentrated under reduced pressure to give crude product. The crude product was purified by Prep-HPLC and lyophilized to give N-[4-(cyclopropanesulfonyl)phenyl]-6,7-diethoxyisoquinolin-1-amine (32) as a white solid (103.1 mg, 59.40% yield). ¹H NMR (400 MHz, DMSO-d₆): δ 9.33 (s, 1H) 8.03 (d, J=8.88 Hz, 2H) 7.94 (d, J=5.63 Hz, 1H) 7.74-7.83 (m, 3H) 7.29 (s, 1H) 7.21 (d, J=5.63 Hz, 1H) 4.22 (dq, J=18.79, 6.95 Hz, 4H) 2.68-2.88 (m, 1H) 1.32-1.55 (m, 6H) 0.93-1.20 (m, 4H).

### 6-ethoxy-7-(2-fluoroethoxy)-N-(4-methanesulfonylphenyl)isoquinolin-1-amine (33)

The above compound was prepared according to Scheme 49 under the specific conditions below:

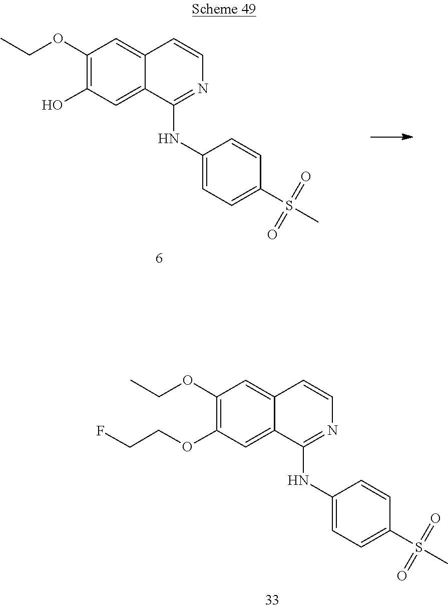 [SMILES: CCOc1cc2ccnc(Nc3ccc(S(C)(=O)=O)cc3)c2cc1O ; CCOc1cc2ccnc(Nc3ccc(S(C)(=O)=O)cc3)c2cc1OCCF]

To a solution of 6 (500 mg, 1.33 mmol) in dimethylformamide (5 mL) was added K₂CO₃ (549.51 mg, 3.98 mmol) and 1-fluoro-2-iodo-ethane (276.66 mg, 1.59 mmol) at 20° C. The mixture was stirred at 60° C. for 3 hours. LCMS showed the starting material was consumed completely, the desired product was detected. The reaction mixture was diluted with brine (10 mL) and extracted with ethyl acetate (3×5 mL). The combined organic layers were washed with brine (2×5 mL), dried over Na₂SO₄, filtered and concentrated under reduced pressure. The residue was purified by prep-HPLC and lyophilized to give 6-ethoxy-7-(2-fluoroethoxy)-N-(4-methanesulfonylphenyl)isoquinolin-1-amine (33) as a white solid (187.0 mg, 99.8% purity). ¹H NMR (400 MHz DMSO-d₆): δ=9.34 (s, 1H), 8.04 (d, J=9.0 Hz, 2H), 7.96 (d, J=5.6 Hz, 1H), 7.86-7.80 (m, 3H), 7.33 (s, 1H), 7.23 (d, J=5.8 Hz, 1H), 4.95-4.78 (m, 2H), 4.51-4.39 (m, 2H), 4.22 (q, J=7.0 Hz, 2H), 3.16 (s, 3H), 1.43 (t, J=7.0 Hz, 3H).

### 6,7-bis(2-fluoroethoxy)-N-(4-methanesulfonylphenyl)isoquinolin-1-amine (34)

The above compound was prepared according to Scheme 50 under the specific conditions below:

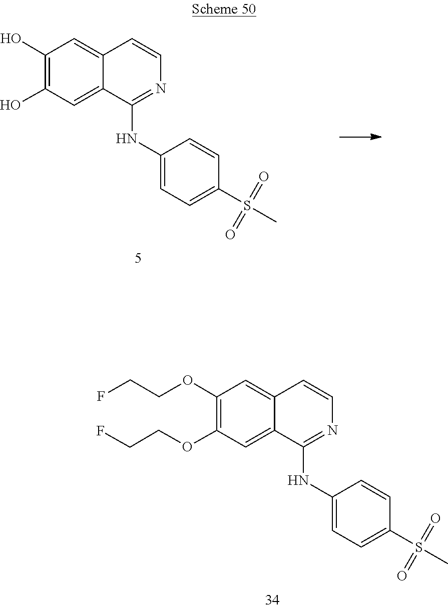 [SMILES: CS(=O)(=O)c1ccc(Nc2nccc3cc(O)c(O)cc23)cc1 ; CS(=O)(=O)c1ccc(Nc2nccc3cc(OCCF)c(OCCF)cc23)cc1]

To a solution of 5 (250 mg, 718.92 μmol) in dimethylformamide (3 mL) was added K₂CO₃ (298.08 mg, 2.16 mmol) and 1-fluoro-2-iodo-ethane (275.14 mg, 1.58 mmol) at 20° C. The mixture was stirred at 60° C. for 3 hours. LCMS showed the starting material was consumed completely, the desired product was detected. The reaction mixture was diluted with brine 10 mL and extracted with ethyl acetate (3×5 mL). The combined organic layers were washed with brine (2×5 mL), dried over Na₂SO₄, filtered and concentrated under reduced pressure to give a residue. The residue was purified by prep-HPLC and lyophilized to give 6,7-bis(2-fluoroethoxy)-N-(4-methanesulfonylphenyl)isoquinolin-1-amine (34) as a grey solid (137.4 mg, 99.7% purity). ¹H NMR (400 MHz DMSO-d₆): δ=9.36 (s, 1H), 8.07-8.03 (m, 2H), 7.97 (d, J=5.6 Hz, 1H), 7.88 (s, 1H), 7.86-7.82 (m, 2H), 7.39 (s, 1H), 7.22 (d, J=5.6 Hz, 1H), 4.92 (dt, J=3.8, 7.9 Hz, 2H), 4.80 (dt, J=3.8, 7.9 Hz, 2H), 4.49 (td, J=3.8, 16.4 Hz, 2H), 4.41 (td, J=3.8, 16.4 Hz, 2H), 3.16 (s, 3H).

### 1-[(4-methanesulfonylphenyl)methyl]-6-(propan-2-yloxy)isoquinoline (35)

The above compound was prepared according to Scheme 51 under the specific conditions below:

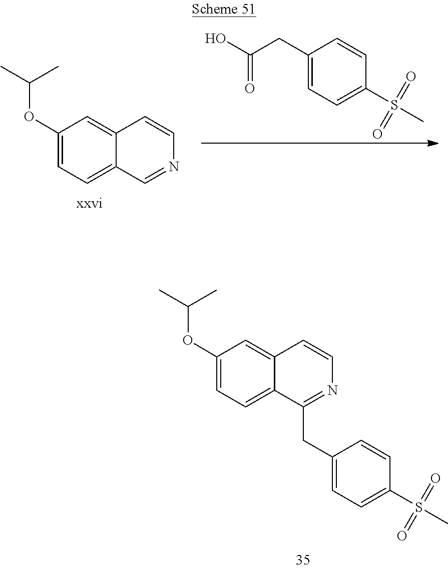 [SMILES: CC(C)Oc1ccc2cnccc2c1 ; CS(=O)(=O)c1ccc(CC(=O)O)cc1 ; CC(C)Oc1ccc2c(Cc3ccc(S(C)(=O)=O)cc3)nccc2c1]

To a solution of 6-(propan-2-yloxy)isoquinoline xxvi (1 g, 4.81 mmol) in CH₃CN (50 mL) was added 2-(4-methylsulfonylphenyl) acetic acid (4.12 g, 19.23 mmol) and [phenyl-(2,2,2-trifluoroacetyl)ox y-iodanyl]2,2,2-trifluoroacetate (4.13 g, 9.61 mmol) at 20° C. under Argon. The mixture was stirred under radiation from 34 W blue LED for 16 hrs. LCMS showed 11% of the starting material was remained and 15% of desired MS was detected. Then the reaction mixture was treated with saturated aqueous NaHCO₃ (30 mL), extracted with ethyl acetate (20 mL×3), washed with brine (20 mL), dried over Na₂SO₄, filtered and the filtrate was concentrated under reduced pressure to give crude product. The crude product was purified by prep-HPLC and lyophilized to give 1-[(4-methanesulfonylphenyl)methyl]-6-(propan-2-yloxy)isoquinoline (35) as a white solid (46.6 mg, 2.64% yield, 96.7% purity). ¹H NMR (400 MHz, DMSO-d₆): δ=8.33 (d, J=5.75 Hz, 1H) 8.23 (d, J=9.13 Hz, 1H) 7.81 (d, J=8.38 Hz, 2H) 7.51-7.63 (m, 3H) 7.35 (d, J=2.50 Hz, 1H) 7.21 (dd, J=9.19, 2.56 Hz, 1H) 4.81 (spt, J=5.98 Hz, 1H) 4.68 (s, 2H) 3.15 (s, 3H) 1.33 (d, J=6.00 Hz, 6H).

### 6-[(4-fluorophenyl)methoxy]-N-(4-methanesulfonylphenyl)isoquinolin-1-amine (36)

The above compound was prepared according to Scheme 52 under the specific conditions below:

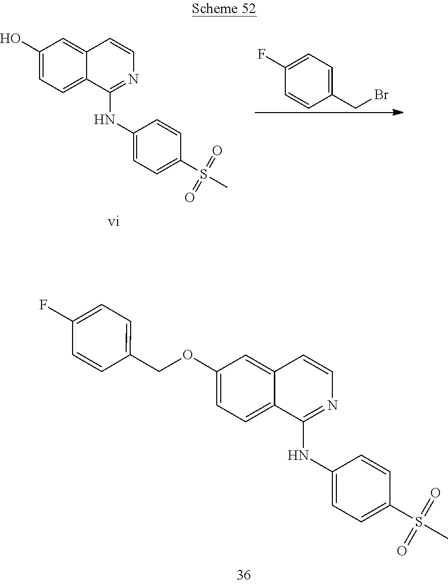 [SMILES: CS(=O)(=O)c1ccc(Nc2nccc3cc(O)ccc23)cc1 ; Fc1ccc(CBr)cc1 ; CS(=O)(=O)c1ccc(Nc2nccc3cc(OCc4ccc(F)cc4)ccc23)cc1]

To a solution of vi (200 mg, 572.59 μmol) in DMF (2 mL) was added K₂CO₃ (158.28 mg, 1.15 mmol) and 1-(bromomethyl)-2-methyl-benzene (127.16 mg, 687.11 μmol, 92.08 μL) at 20° C. The reaction was stirred for 16 hours at 60° C. LCMS showed the reaction was completed. The reaction was diluted with water (10 mL) and extracted with ethyl acetate (3×10 mL). The organic layer was washed with brine (10 mL), dried over Na₂SO₄, filtered and concentrated under reduced pressure. The residue was purified by pre-HPLC and lyophilized to give 6-[(4-fluorophenyl)methoxy]-N-(4-methanesulfonylphenyl)isoquinolin-1-amine (36) as a grey solid (136 mg, 99.8% purity). ¹H NMR (400 MHz, DMSO-d₆): δ=9.55 (s, 1H) 8.48 (d, J=9.25 Hz, 1H) 8.12 (d, J=8.88 Hz, 2H) 8.03 (d, J=5.75 Hz, 1H) 7.82 (d, J=8.88 Hz, 2H) 7.58 (dd, J=8.63, 5.63 Hz, 2H) 7.42 (d, J=2.50 Hz, 1H) 7.35 (dd, J=9.26, 2.50 Hz, 1H) 7.22-7.30 (m, 3H) 5.26 (s, 2H) 3.16 (s, 3H).

### 6-ethoxy-N-(4-methanesulfonylphenyl)-7-propylisoquinolin-1-amine (37)

The above compound was prepared according to Scheme 53 under the specific conditions below:

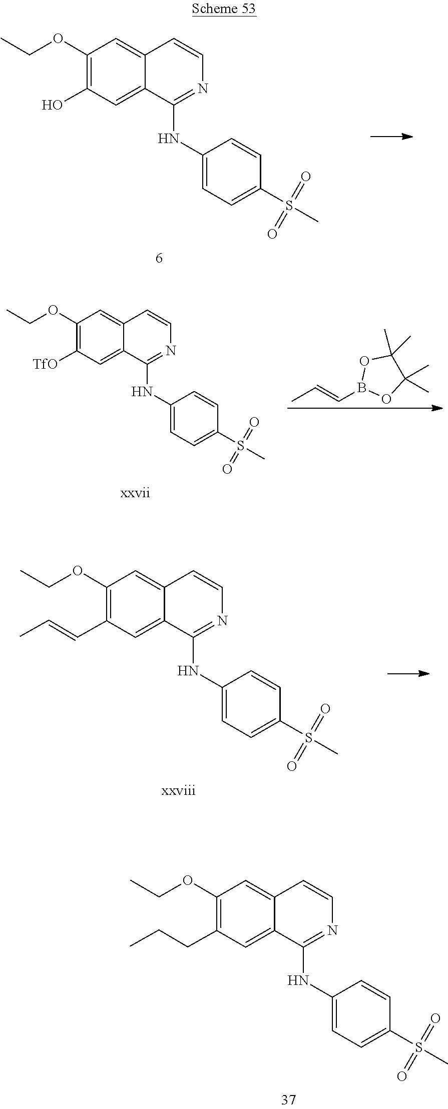

To a solution of 6 (1.2 g, 3.18 mmol) in dichloromethane (4 mL) was added trimethylamine (1.61 g, 15.90 mmol, 2.21 mL) and a solution of trifluoromethylsulfonyl trifluoromethanesulfonate (1.79 g, 6.36 mmol, 1.05 mL) in dichloromethane (1 mL) at 0° C. The mixture was stirred at 20° C. for 3 hours. LCMS showed the starting material was consumed completely, the desired product was detected. The reaction mixture was diluted with brine (10 mL) and extracted with ethyl acetate (3×5 mL). The combined organic layers were washed with brine (2×5 mL), dried over Na₂SO₄, filtered and concentrated under reduced pressure to give a residue. The residue was purified by prep-TLC (petroleum ether:ethyl acetate=1:2) to give xxvii (500 mg, 28.85% yield) as a white solid. ¹H NMR (400 MHz DMSO-d₆): δ=9.62 (s, 1H), 8.71 (s, 1H), 8.20-8.01 (m, 3H), 7.91-7.82 (m, 2H), 7.64 (s, 1H), 7.30 (d, J=5.8 Hz, 1H), 4.38-4.28 (m, 2H), 3.19-3.15 (m, 3H), 1.43 (t, J=7.0 Hz, 3H).

To a solution of xxvii (300 mg, 550.49 μmol) and 4,4,5,5-tetramethyl-2-[(E)-prop-1-enyl]-1,3,2-dioxaborolane (277.51 mg, 1.65 mmol) in tetrahydrofuran (4 mL) and H₂O (1 mL) was added ditert-butyl(cyclopentyl)phosphane; dichloropalladium; iron (35.88 mg, 55.05 μmol) and K₃PO₄ (233.70 mg, 1.10 mmol) at 20° C. under N₂. The mixture was stirred at 80° C. for 3 hours. LCMS showed the starting material was consumed completely, the desired product was detected. The reaction mixture was diluted with brine (10 mL) and extracted with ethyl acetate (3×5 mL). The combined organic layers were washed with brine (2×5 mL), dried over Na₂SO₄, filtered and concentrated under reduced pressure to give a residue. The residue was purified by prep-HPLC and lyophilized to give xxviii (90 mg, 41.25% yield) as a white solid. ¹H NMR (400 MHz DMSO-d₆): δ=9.53 (s, 1H), 8.52 (s, 1H), 8.10 (d, J=8.9 Hz, 2H), 7.96 (d, J=5.6 Hz, 1H), 7.83 (d, J=8.9 Hz, 2H), 7.25 (s, 1H), 7.20 (d, J=5.8 Hz, 1H), 6.78 (dd, J=1.4, 15.9 Hz, 1H), 6.68-6.54 (m, 1H), 4.20 (q, J=6.9 Hz, 2H), 3.16 (s, 3H), 1.95 (dd, J=1.1, 6.4 Hz, 3H), 1.44 (t, J=6.9 Hz, 3H).

To a mixture of Pd/C (24.17 mg, 22.71 μmol) in methyl alcohol (1 mL) was added a solution of xxviii (90 mg, 227.07 μmol) in methyl alcohol (1 mL) under H₂ atmosphere. The suspension was degassed and purged with H₂ for 3 times. The mixture was stirred under H₂ (15 Psi) at 20° C. for 2 hours. LCMS showed the starting material was consumed completely, the desired product was detected. The reaction mixture was filtered and the filtrate was concentrated under reduced pressure to give 6-ethoxy-N-(4-methanesulfonylphenyl)-7-propylisoquinolin-1-amine (37) as a white solid (64.4 mg, 97.2% purity). ¹H NMR (400 MHz DMSO-d₆): δ=9.44 (s, 1H), 8.26 (s, 1H), 8.09 (d, J=8.9 Hz, 2H), 7.97 (d, J=5.8 Hz, 1H), 7.82 (d, J=8.9 Hz, 2H), 7.26-7.19 (m, 2H), 4.19 (q, J=7.0 Hz, 2H), 3.15 (s, 3H), 2.77-2.71 (m, 2H), 1.69 (sxt, J=7.5 Hz, 2H), 1.42 (t, J=6.9 Hz, 3H), 0.96 (t, J=7.4 Hz, 3H).

### Example 2. In Vitro Effects of Compounds of the Invention in Enhancing Glucose Uptake and Lactate Levels Secretion

To assess for the effect of the compounds of the invention, those were tested in primary mouse astrocytes and astrocytes differentiated from human-derived induced pluripotent stem cells (iPSCs). Secretion of lactate was measured indirectly through the acidification of extracellular medium using extracellular pH sensor SNARE-5F-(AND-6)-CAR (SNARF5) as described below.

Table 1 shows the activity of compounds of the invention on the extracellular medium acidification (SNARF5) assay in astrocytes in vitro, which indicates their glycolytic activity and their ability to produce lactate. ‘+’ indicated activity of compounds with EC50>1 μM and ‘++’ indicates activity of compounds with EC50<1 μM.

TABLE 1

| Compound | Activity |
|---|---|
| 1 | ++ |
| 2 | ++ |
| 3 | ++ |
| 4 | ++ |
| 5 | ++ |
| 6 | + |
| 7 | + |
| 8 | ++ |
| 9 | ++ |
| 10 | ++ |
| 11 | ++ |
| 12 | ++ |
| 13 | + |
| 14 | ++ |
| 15 | + |
| 16 | ++ |
| 17 | ++ |
| 18 | ++ |
| 19 | ++ |
| 20 | ++ |
| 21 | ++ |
| 22 | ++ |
| 23 | ++ |
| 24 | ++ |
| 25 | ++ |
| 26 | ++ |
| 27 | ++ |
| 28 | ++ |
| 29 | ++ |
| 30 | ++ |
| 31 | ++ |
| 32 | ++ |
| 33 | ++ |
| 34 | ++ |
| 35 | ++ |
| 36 | ++ |
| 37 | ++ |

### Primary Mouse Cell Cultures

Primary cultures of cerebrocortical astrocytes were obtained from 1 to 2-day-old OF1 mouse pups (Charles River Laboratories). Briefly, cortices were isolated and minced in small pieces under a dissecting microscope. The cells were incubated for 30 min at 37° C. in a solution containing 20 U/ml papain, 1 mM L-cysteine and 10 kU/ml DNase I. After dissociation, papain activity was stopped by the addition of fetal calf serum (FCS). Single-cell suspension was then obtained by mechanical dissociation, which consisted in cells trituration in a DMEM D7777 medium supplemented with 44 mm NaHCO₃, 10 ml/L antibiotic/antimycotic solution and 10% FCS. The cells were seeded at an average density of ˜10′000 cells/cm² on poly-D-lysine coated 96- or 12-well culture plates, depending on their use, and grown in DMEM D7777 medium supplemented with 44 mm NaHCO₃, 10 ml/L antibiotic/antimycotic solution and 10% FCS at 37° C. in a humidified atmosphere containing 5% CO2/95% air. Culture medium was renewed twice a week. Cells were stimulated and harvested between DIV14 and DIV17, when confluence and cell growth were optimal.

### Primary Mouse Astrocyte Neuron Co-Cultures

Primary mouse neuronal cultures were obtained from 18-day OF1 mouse embryos (Charles River Laboratories). Briefly, cortices were isolated and minced in small pieces under a dissecting microscope. The cells were incubated for 30 min at 37° C. in a solution containing 20 U/ml papain, 1 mM L-cysteine and 10 kU/ml DNase I. After dissociation, papain activity was stopped by the addition of fetal calf serum (FCS). Single-cell suspension was then obtained by mechanical dissociation, which consisted in cells trituration in a Neurobasal+B-27+Glutamax medium. The cells were seeded at an average density of ˜1.5×10⁵ cells/cm² on poly-D-lysine coated 12-well culture plates and grown in Neurobasal medium supplemented with B-27 and Glutamax at 37° C. in a humidified atmosphere containing 5% CO2/95% air. Neurons were used at DIV10. Primary mouse astrocytes were cultured as described earlier, except that they were grown on 15 mm diameter Nunc Thermanox coverslips with two 3-mm paraffin beads on each of them. At the day of experiment, the co-culture was initiated by transferring the coverslips in each well of 12-well plates neuronal cultures, with astrocytes on coverslips facing neurons in the well, being separated by 3 mm paraffin beads.

### Extracellular Medium Acidification (SNARF5)

Secretion of lactate was measured indirectly through the acidification of extracellular medium using extracellular pH sensor SNARF-5F-(AND-6)-CAR (SNARF5). After washing cells twice with stimulation medium (DMEM (D5030, Sigma), 1 mM NaHCO₂, and 5 mM Glucose, pH 7.4) at 37° C., cells were stimulated with compounds at a final concentration ranging from 10 nM to 30 μM in 50 μl per well of stimulation medium supplemented with 10 μM of SNARF5 (Life Technologies Corporation). Each compound was tested in two different plates for duplicates. After 30, 60 and 90 min stimulation, fluorescence was read at exc. (excitation) 480 nm/emm. (emission) 580 nm and at exc 480 nm/emm. 630 nm. The ratio of fluorescence between 630 nm and 580 emission values, which is proportional to extracellular pH, was calculated. In each plate, 8 wells were used for negative controls (DMSO 0.1%) and 8 wells were used for positive controls (CCCP 2 μM in DMSO). Results are shown as % of positive control effect (0% and 100% being activities of the Veh and positive control, respectively). Medium acidification from primary astrocytes treated with compounds of the invention are shown in Table 1.

### Extracellular Lactate Quantification

Secretion of L-lactate was measured in the extracellular medium of 96-well plated astrocytes after 90 min stimulation (at 37° C., in 5% CO₂/95% air conditions) with Vehicle (DMSO), the compounds of the invention (100 nM to 100 μM) or positive control. The positive control consisted in carbonyl cyanide m-chlorophenyl hydrazine (CCCP, 2 μM), an inhibitor of mitochondrial oxidative phosphorylation that hence leads to enhanced glycolysis and secretion of lactate. Stimulation medium was composed of D5030 medium complemented with 5 mM D-glucose and 44 mM sodium bicarbonate, pH 7.2. To quantify lactate concentrations in the extracellular medium, 200 μl of a 0.2M Glycine-semicarbazide buffer (pH 10) containing 3 mM NAD and 14 U/ml LDH was added to each well of a 96-well plate containing 30 μl aliquots of extracellular medium. Samples were incubated at 37° C. for 1 h. Fluorescence intensity (340 nm excitation/450 nm emission), which represents the amount of NADH produced, was measured, and lactate concentration values were determined relative to a standard curve of L-lactate concentrations.

### 2-Deoxylucose (2DG) Uptake

Astrocytes grown on 12-well plates and transfected with either scramble siRNA or GLUT1 siRNA were used. 1 day after having replaced transfection medium (DIV13), 2DG uptake was measured after treatment for 30 min with Vehicle (0.1% DMSO) or with the compound of the invention (1) to (5) (concentrations of 0.1 to 10 μM). During treatment, 1 mM 2DG was added to the medium for assessment of 2DG uptake. At the end of the stimulation, medium was removed and replaced with 150 μl NaOH 0.1M, and stored at −20° C. After thawing, cells were collected using cell scraper and heated for 40 min at 85° C. Then, 150 ul HCl 0.1M and TAE buffer 200 mM was added to each condition. 20 μl were added to a transparent 96-well plate and 2DG was quantified by addition of a reaction solution containing 50 mM TAE, 50 mM KCl, 0.02% BSA, 0.1 mM NADP, 0.2 U/ml diaphorase, 2 mM resazurin and 20 U/ml glucose-6-phosphate dehydrogenase. Concentration of 2DG in samples were calculated by comparison with standard curve of deoxy-glucose-6-phophate ranging from 0 to 1 nmoles.

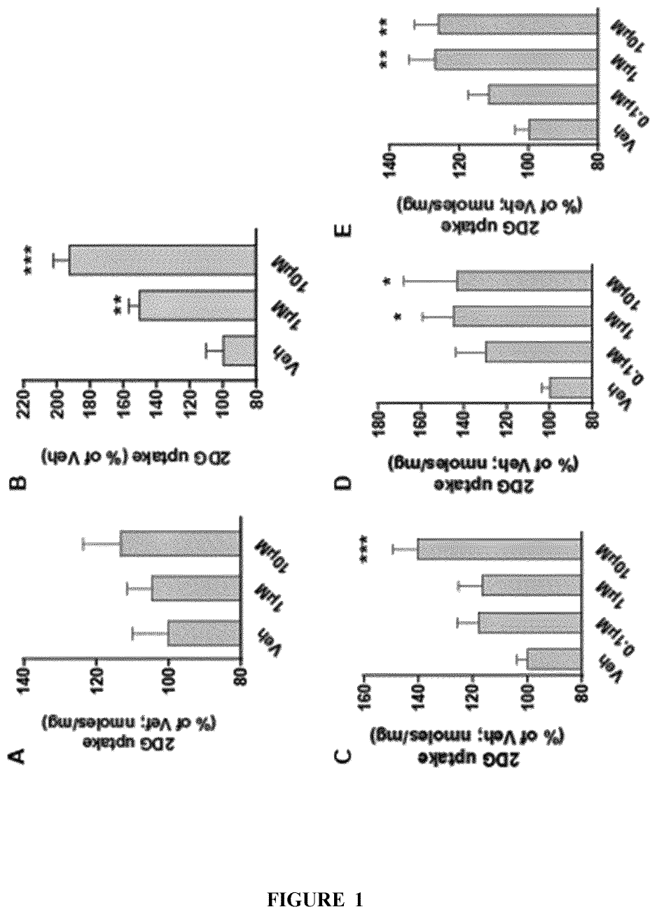

Uptake of 2DG was analyzed in primary astrocytes after treatment with compounds (1) to (5) (FIG. 1 A to E, respectively) and it was observed that these compounds significantly enhance the uptake of 2DG in astrocytes.

### MTT Mitochondrial Activity Assay in Primary Astrocytes

To monitor mitochondrial activity in astrocytes, which is linked to the metabolic process of glycolysis and production of lactate, astrocytes in 96-well plates were stimulated for 24 h (37° C. 5% CO₂/95% air) with the compounds of the invention ranging from 10 nM to 10 μM.

After stimulation, 5 mg/ml thiazol blue tetrazolium bromide (MTT) in D5030 medium complemented with 5 mM D-glucose and 44 mM sodium bicarbonate (pH 7.2) was added to each well, and cells were incubated for 4 h at 37° C. (5% CO₂). The medium was then removed and the amount of reduced MTT, i.e formazan, solubilized in DMSO (50 μl/well) was determined using a spectrophotometer (absorbance of 570 nm).

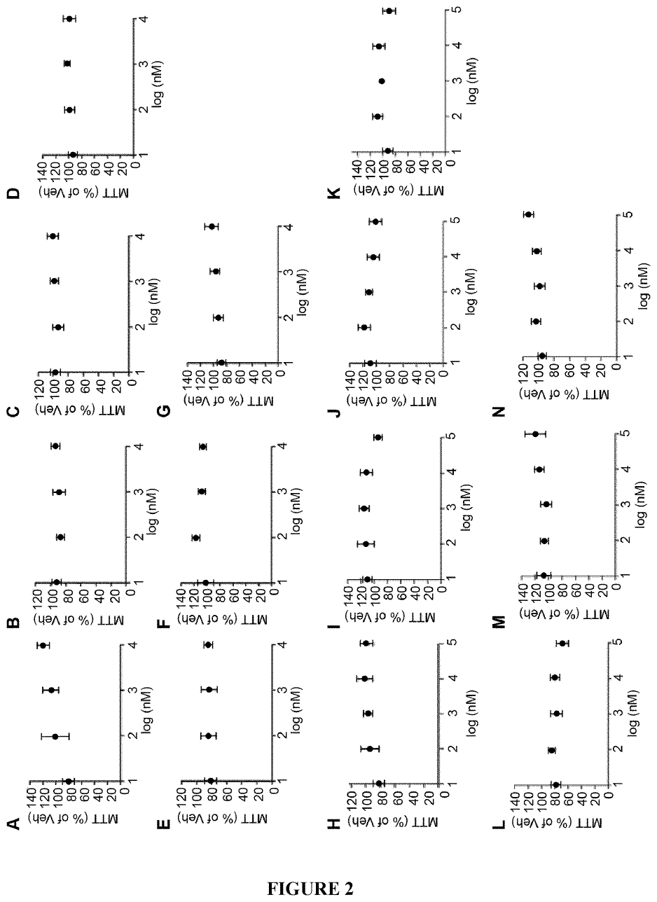

The mitochondrial activity was monitored in MTT colorimetric assay in primary astrocytes treated with Compounds (1), (2), (3), (4) (5), (9) and (10) at different concentrations after 1.5 h (FIG. 2 A, B, D, E, F, C, H, respectively) or 24 h (FIG. 2 H, I, K, L, M, J, N, respectively). These data indicate that none of the selected compounds had direct effect on mitochondrial activity in astrocytes.

MTT Mitochondrial in Neurons from Neurons Pure Culture and Neurons from Astrocytes-Neuron Co-Cultures

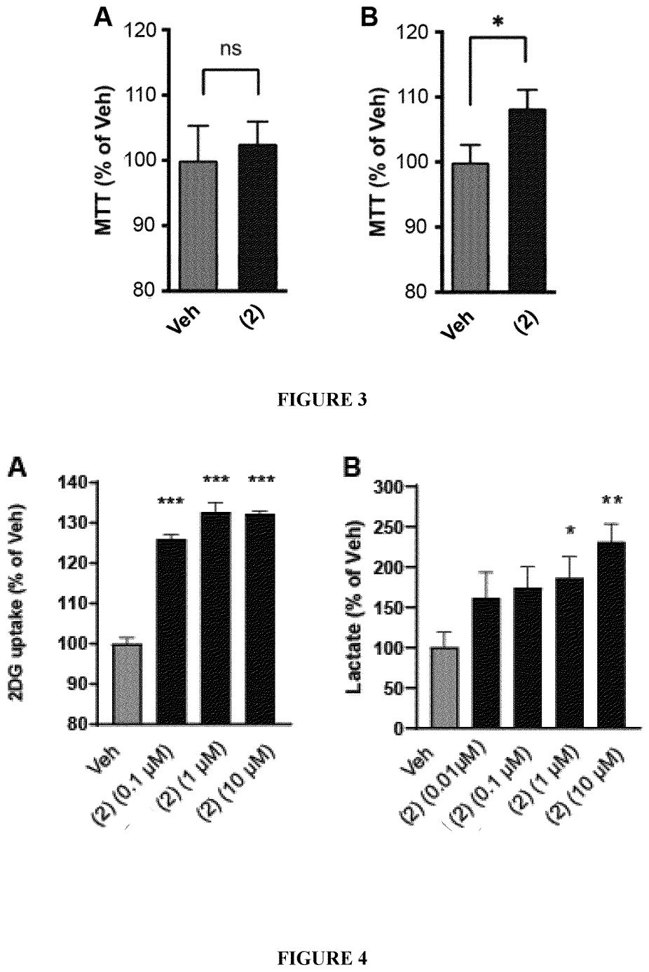

Neuron pure culture or astrocyte-neuron co-cultures were treated for 2 hours with compound (2) at 10 μM in a solution composed of Neurobasal complemented with B-27 and Glutamax, as well as 0.25 mg/ml thiazol blue tetrazolium bromide (MTT). Neuron pure culture were treated with addition of empty coverslips, while neurons from co-cultures were treated in addition of astrocytes grown on coverslips, as described earlier. After stimulation, empty coverslips (neuron pure cultures) or astrocytes on coverslips (astrocyte-neuron co-cultures) were separated from neurons, medium was removed and the amount of reduced MTT, i.e formazan, solubilized in DMSO (50 μl/well) was determined using a spectrophotometer (absorbance of 570 nm). Results indicate that treatment compound (2) enhances mitochondrial activity of neurons in astrocyte-neuron co-culture (FIG. 3B), but not of neuron in pure culture (FIG. 3A), indicating that the effects of this compound on neurons require the presence of astrocytes.

Human iPSC-Derived Astrocytes

Human iPSC-derived astrocytes were purchased from NCardia (NCyte Astrocytes) and were plated at a density of ˜10′000 cells/cm² in 96-well or 12-well plates, according to the manufacturer's instructions and using the recommended media. Experiments were performed at 7 days in vitro. Cells were treated for 1.5 hours with compound (2). Uptake of 2-DG and release of lactate were quantified as described previously. Compound (2) increased uptake of 2-DG and release of lactate from human iPSC-derived astrocytes (FIGS. 4A and B, respectively).

### Example 3: In Vivo Effects of Compounds of the Invention

To assess the effect of the compounds of the invention on brain extracellular levels of glucose and lactate, they have been tested though the in vivo monitoring of glucose lactate levels after treatment with the compounds of the invention as follows.

### Animals

All experiments were carried out in strict accordance with the Swiss Federal Guidelines for Animal Experimentation and were approved by the Cantonal Veterinary Office for Animal Experimentation (Canton of Geneva, Switzerland). Adult male C57Bl/6J wild-type mice weighting 18-28 g (8 to 12 weeks of age) were used (Charles River Laboratories). Animals were housed in groups of 3-5 in polypropylene cages (30×40×15 cm) with wire mesh top in a temperature (22±2° C.) and humidity (55±15%) controlled environment on a 12-hour light cycle (07.00-19.00 h lights on), except after surgeries when animal were housed individually. The samples (Vehicle or compounds of the invention) were administered per os (gavage) in a solution made of water supplemented with 0.4% hydroxypropyl methylcellulose (HPMC) Methocel 4 KM (w/v) and 0.25% Tween-20 (v/v), as previously described (Thackaberry et al., 2010, Toxicol Sci., 117(2):485-92). Concentrations of the compounds tested ranged from 3 to 30 mg/kg.

### Lactate and Glucose Biosensors

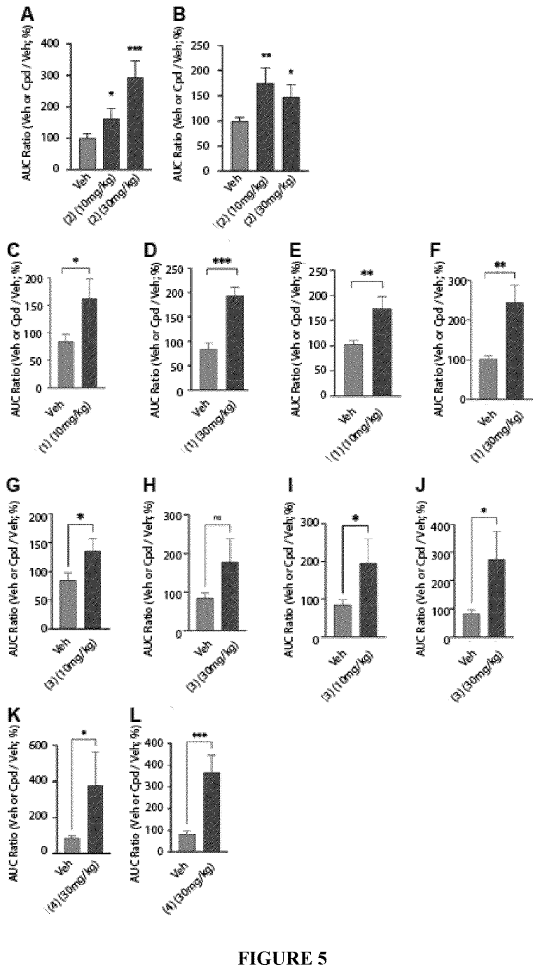

Cerebral extracellular levels of L-lactate and D-glucose were monitored in vivo using lactate and glucose biosensors (Pinnacle Technology), respectively, according to the manufacturer's instructions. Cannulae were surgically implanted in the medial prefrontal cortices (coordinates: −1.0 mm (to bregma), lateral+/−1.0 mm (to mideline), ventral −1.0 mm (to dura))) of isoflurane-anesthetized mice 5 to 7 days prior experiment. After surgery, mice were monitored closely and received analgesic treatment for at least 4 days. After mice had fully recovered from surgery, compounds of the invention of vehicle were administered per os as previously described and cerebral levels of extracellular lactate and glucose were dynamically recorded for 6 hours using biosensors. Mice were administered vehicle alone first, followed 3 hours later by vehicle or Compounds of the Invention. Concentrations of cerebral extracellular lactate and glucose were calculated from the biosensor electric signals using post-calibration values. Each signal of lactate or glucose fluctuation after compound (or vehicle) administration was expressed as a fold change relative to the lactate or glucose fluctuation following the first administration of vehicle alone, each animal hence being its own control. Area Under the Curve (AUC) of lactate and glucose concentration curves were calculated using Graphad Prism and the ratio of AUC after drug over Vehicle administration was calculated. Extracellular concentrations of lactate and glucose were measured in real time in freely moving animals for 3 hours after administration of Vehicle or compounds (1) to (4) at doses ranging from 10 to 30 mg/kg (FIG. 5). Results indicate that treatment with compound (1) (FIG. 5C, D), compound (2) (FIG. 5A), compound (3) (FIG. 5G, H) and compound (4) (FIG. 5K) significantly increases extracellular glucose levels in the brain of treated mice at different doses, as compared to vehicle. Results indicate that treatment with compound (1) (FIG. 5E, F), compound (2) (FIG. 5B), compound (3) (FIG. 51, J), compound (4) (FIG. 5L) significantly increases extracellular lactate levels in the brain of treated mice at different doses, as compared to vehicle.

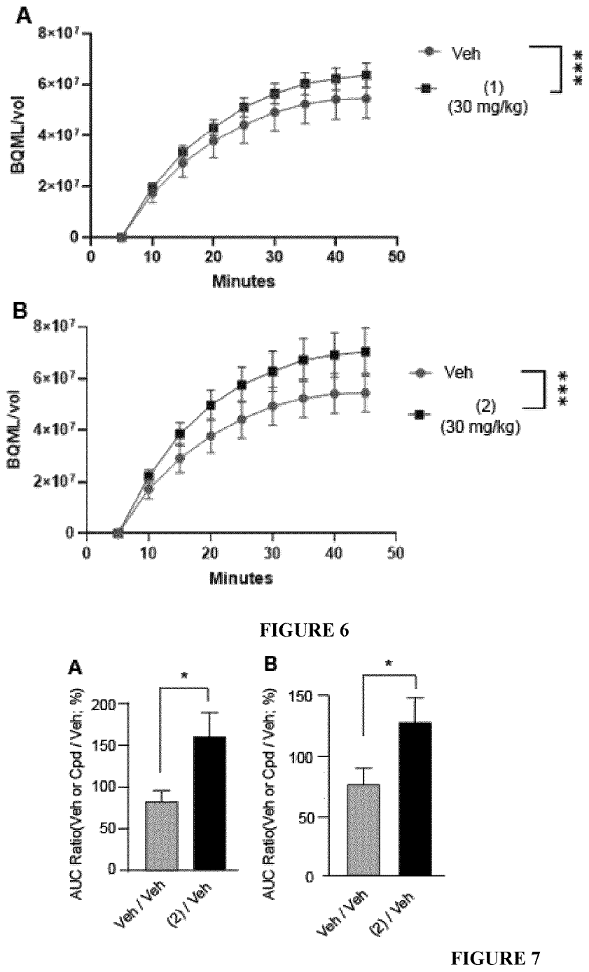

¹⁸F-FDG PET Glucose uptake in the brain was measured in vivo using 18F-fluorodeoxyglucose (FDG) with positron emission tomography (PET) Scan. Mice were fasted for 12 hours before the scans PET/CT measurements. Compounds of the invention were administered by gavage, followed by an intraperitoneal injection of 5 MBq of ¹⁸F-FDG in 300 μL of saline. 20 minutes after injection, mice were anesthetized and placed in the microPET/CT scanner bed. The microPET scan lasted for 40 minutes with an acquisition every 5 minutes and was followed by a 5-minute CT scan for positioning. At the end of the imaging protocol, the animals were returned to their cage and a second measurement was performed seven days later. At the end of the second measurement mice, were sacrificed. Blood and brain were collected. Quantification in of ¹⁸F-FDG in the whole brain (BQML/vol) showed that administration of compounds (1) and (2) at 30 mg/kg increases glucose uptake in the brain as compared to Vehicle (FIGS. 6A and B, respectively).

Altogether, these data support that compounds of the invention enhance glucose uptake and intracerebral glucose and lactate levels in the brain.

### Example 4: In Vivo Effects of Compounds of the Invention in Preclinical Models of Hypometabolic Diseases, Including GLUT1-DS

To assess for the effect in vivo of the compounds of the invention, those were tested in the following model.

### Animals

Heterozygous transgenic mice on 129/SvJ genetic background in which glucose transporter 1 (GLUT1) has been knocked down were used (GLUT1(+/−)) (Wang et al., 2016, Human Molecular Genetics, 15(7)). GLUT1-deficiency syndrome (GLUT1-DS) is a prototypic hypometabolic diseases caused by the mutation of GLUT1 and decreased levels of glucose and lactate in the brain (Tang et al., 2019, Annals of Clinical and Translational Neurology 6(9)). Mating colonies were composed of wild-type female mice and GLUT1-DS male mice, or GLUT1-DS female mice and wild-type male mice. F1 pups were genotyped after ear punching at weaning using PCR to determine genotype. Mice of 2 to 3 months of age were used.

### Lactate and Glucose Biosensors

Cerebral extracellular levels of lactate and glucose were monitored in vivo in GLUT1-DS transgenic male mice and wild-type (WT) male littermates using lactate and glucose biosensors (Pinnacle Technology), according to the manufacturer's instructions. GLUT1-DS transgenic mice and wild-type littermates, cannulae were surgically implanted in the motor cortex M1/M2 (coordinates: +1.94 mm (to bregma), lateral −1.4 mm (to mideline), ventral −1.0 mm (to dura)). After surgery, mice were monitored closely and received analgesic treatment for at least 4 days. After mice had fully recovered from surgery, compounds of the invention or vehicle were administered per os as previously described and cerebral levels of extracellular lactate or glucose were dynamically recorded for 6 hours using lactate or glucose biosensors, respectively. Mice were administered orally vehicle alone first, followed 3 hours later by vehicle or compound (2) at a dose of 10 mg/kg. During recording, mice were exposed to novel objects in their cages that consisted in coloured plastic building blocks to stimulate their activity. Concentration of cerebral extracellular lactate or glucose was calculated from lactate or glucose probe electric signals, respectively, using post-calibration values, as described by manufacturer. Area Under the Curve (AUC) of lactate or glucose concentration curves were calculated using Graphpad Prism and the ratio of AUC after Compound of the invention over Vehicle administration was calculated. AUC signals of lactate or glucose after vehicle or compound (2) administration were expressed as fold change relative to AUC of lactate or glucose fluctuation following the first administration of vehicle (FIG. 7). Results indicate that treatment with compound (2) at 10 mg/kg significantly increases extracellular lactate (FIG. 7A) and glucose levels (FIG. 7B) in the brain of treated GLUT1-DS transgenic mice, as compared to vehicle.

### Rotarod

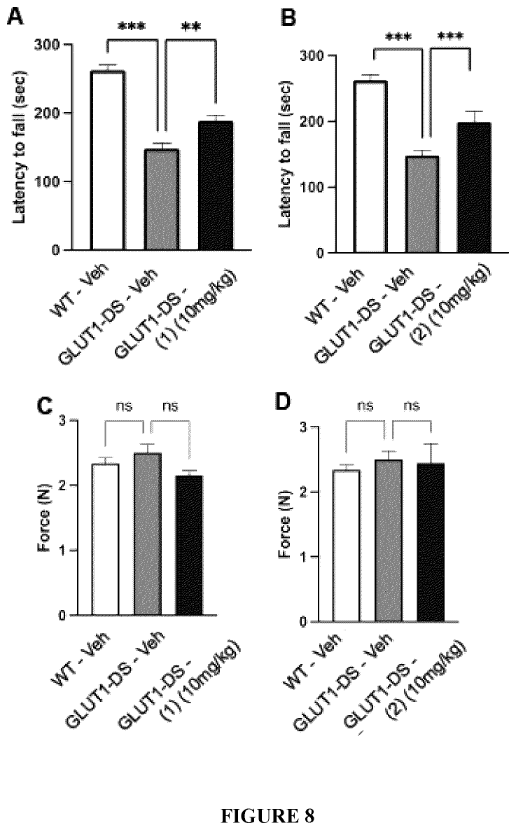

Motor function was measured in wild-type (WT) and GLUT 1-DS transgenic mice in the rotarod after single administration of Veh. or compound (1) or (2) (10 mg/kg) 20 min before rotarod. Rotarod testing consisted in placing the mouse on an accelerating rotating rod (4 to 40 r.p.m.) during a maximum of 300 seconds. 3 consecutive sessions with 15 min apart were performed. Latency for the mouse to fall from the rod was recorded and the maximal latency over the 3 trials was used as a measure of motor function. Mice that did not fall for a duration of 300 seconds were removed and test was terminated with scoring a maximum of 300 seconds. Data indicate that GLUT1-DS transgenic mice had poor performance on the rotarod compared to WT mice, while administration of compound (1) or (2) at a dose of 10 mg/kg increased rotarod performance in GLUT1-DS mice (FIGS. 8 A and B, respectively).

### Grip Strength Test

Motor strength was measured on the grip strength test (Bioseb) after rotarod session. Wild-type (WT) and GLUT1-DS transgenic mice were placed with 4 paws on the grid and maximal grip strength was recorded over 2 sessions with 5 minutes apart. Maximal grip strength over the 2 sessions was recorded. Data indicate that Veh or compound (1) or (2) treatment (10 mg/kg) did not lead to any difference on muscle strength, but rather on muscle coordination as assessed by the rotarod (FIGS. 8 C and D, respectively).

### Example 5: In Vivo Effects of Compounds of the Invention in Preclinical Models of Hypometabolic Diseases, Including Alzheimer's Disease

The effects of compounds of the invention were tested in the above animal model as described below.

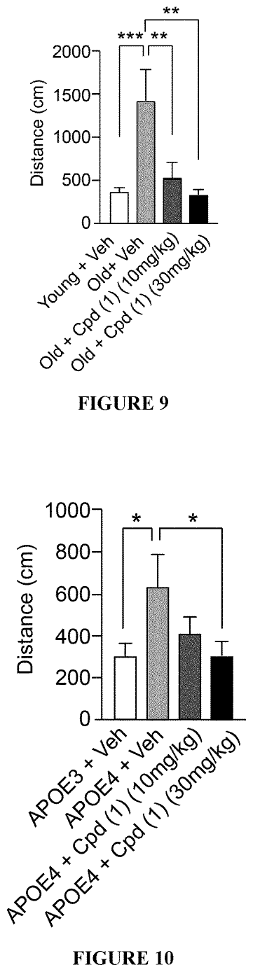

Adult male C₅₇Bl/6J wild-type from 3 month of age (young adults) to 16 month of age (old adults) are used (Charles River). Animals are housed in groups of 3-5 in polypropylene cages (30×40×15 cm) with wire mesh top in a temperature (22±2° C.) and humidity (55±15%) controlled environment on a 12-hour light cycle (07.00-19.00 h lights on). The samples (Vehicle or compounds of the invention) are administered per os (gavage) in a solution made of water supplemented with 0.4% hydroxypropyl methylcellulose (HPMC) Methocel 4 KM (w/v) and 0.25% Tween-20 (v/v), as previously described (Thackaberry et al., 2010, Toxicol Sci., 117(2):485-92). Memory of young adults treated with Vehicle or Old adults treated with Vehicle or compound (1) at 10 mg/kg or 30 mg/kg is tested in the Morris Water Maze, as described below. Treatment is administered at each day of training, 30 minutes before the first training session. No treatment is administered during memory retention test. Data indicate that memory of old mice treated with Vehicle is significantly lower than memory of young mice at 1 day after training, and that treatment of old mice with compound (2) at 10 mg/kg or 30 mg/kg lead to significant increase of memory, as assessed in the Morris Water Maze (FIG. 9).

### APOE4(+) Mouse Model of Alzheimer's Disease

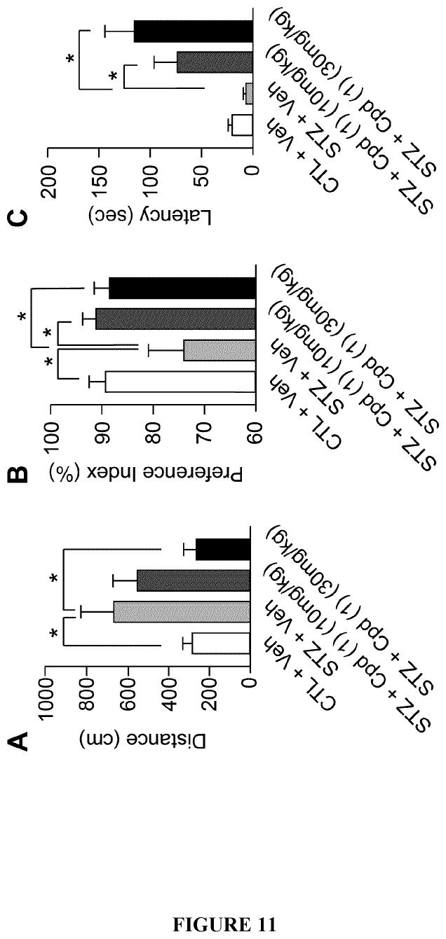

Mouse models carrying the APOE4 human allele have a decrease in metabolic gene expression and cerebral glucose uptake compared to APOE3-expressing model (Williams et al., 2020, neurobiol dis, 136:104742; Lin et al., 2015, J Cereb Blood Flow Metabl, 37(1):217-226; Alata et al., 2015, J Cereb Blood Flow Metab, 35(1):86-94. These physiological perturbations induce a brain hypometabolism, which recapitulates brain metabolic status observed in AD carriers of APOE4 allele. Male and female APOE4(+) and APOE3(+) (controls) transgenic mice are used at 3 months of age. Memory of APOE3(+) mice treated with Veh and APOE4 (+) mice treated with Veh or compound (2) at 10 mg/kg and 30 mg/kg is tested in the Morris Water Maze, as described below. Treatment is administered at each day of training, 30 minutes before the first training session. No treatment is administered during memory retention test. Data indicate that memory of APOE4(+) mice treated with Vehicle is lower than memory of APOE3(+) mice at 7 days after training, and that treatment of APOE4(+) mice with compound (2) at 30 mg/kg leads to significant increase of memory, as assessed in the Morris Water Maze (FIG. 10).

### Streptozotocin Intracerebroventricular (ICV) Injection Model of Alzheimer's Disease

Craniotomies are performed on isoflurane-anesthetized mice for injections of Veh or streptozotocin in cerebral ventricles (coordinates: −0.5 mm (to bregma), lateral+/−1.0 mm (to midline), ventral −2.2 mm (to dura)) (Kelliny S. et al., Molecular Neurobiology, 2021). A solution composed of Veh (0.9% NaCl) or streptozotocin (5 mg/kg) prepared in 1 μl artificial cerebrospinal fluid solution (aCSF, 20 mM citrate buffer, pH 4.2) is injected in both cerebral ventricles using Hamilton micro syringe at a constant speed of 100-200 nl/min. After injection, skin of the skull is sutured, and mice is monitored closely and receives analgesic treatment for at least 4 days. After mice had fully recovered from surgery, memory tests including Morris Water Maze (MWM), Inhibitory Avoidance (IA) and Novel Object Recognition (NOR), as described below, are performed. Memory of mice injected with Veh and treated with Veh, or injected with streptozotocin and treated with either Veh or compound (2) at 10 mg/kg and 30 mg/kg was tested in the Morris Water Maze, novel object recognition and inhibitory avoidance, as described below. In the Morris Water Maze, treatment was administered at each day of training, 30 minutes before the first training session. In the Novel object recognition and Inhibitory Avoidance tests, treatment was administered one time at 30 min before the acquisition trial. Data indicate that memory of mice injected with streptozotocin and treated with Vehicle is weaker than memory of mice injected with saline and treated with Vehicle in the Morris Water Maze, Novel Object Recognition, and Inhibitory Avoidance memory tasks (FIGS. 11 A, B and C, respectively). Data also indicate that memory of mice injected with streptozotocin and treated with compound (2) at 30 mg/kg is significantly higher than those treated with Veh in the Morris Water Maze at 1 Day after training (FIG. 11A), and that memory of mice injected with streptozotocin and treated with compound (2) at 10 mg/kg and 30 mg/kg is significantly higher than those treated with Veh in the Novel object recognition at 1 day after training (FIG. 11B) and in the Inhibitory Avoidance at 1 day after training (FIG. 11C).

### Inhibitory Avoidance

Inhibitory avoidance (IA) test is a well-established memory paradigm in rodents that measures contextual memory associated with a mild electrical footshock in a specific context (the dark compartment of the IA chamber). Each mouse is handled for 5 minutes per day for at least 4 consecutive days to reduce animal's stress due to experimenter's presence/manipulation during test days. Inhibitory avoidance is carried out in an IA chamber (MedAssociates) that consists in a rectangular Perspex box divided into a safe and a shock compartment separated by an automatically operated sliding door. The safe compartment is white and illuminated while the shock compartment is black and dark. Mice are trained for IA 20 min after oral administration of the drug or vehicle. During training, mice are placed into the safe compartment with their heads facing away from the door. After 10 seconds, the door separating the compartments is automatically opened, allowing the mouse to access the shock compartment (which it usually did within 20 sec). The door closes 1 second after the mouse entered the dark compartment, and a 2-second 0.6 mA intensity footshock is delivered to the grid floor of the shock chamber via a constant current scrambler circuit. After footshock delivery, the mouse stays for 10 seconds in the dark compartment and is then returned to its home cage. Memory retention was measured at 24 h after training by placing the mouse back into the lit compartment and recording its latency (in seconds) to enter the dark compartment. No footshock is administered during retention tests. The test is terminated once the mouse entered the dark compartment, or after a 900 second cutoff limit.

### Morris Water Maze

Morris Water Maze (MWM), a well-established spatial and contextual memory test, will be used. MWM consists in 4 consecutive training days, each composed of 4×90 sec training sessions where the mouse learns to locate a hidden platform in a pool. If mouse does not find the platform within the 90 sec training session, it is accompanied by the experimenter to the platform. Mouse is allowed to stay 30 sec on the platform. After training, memory is tested in probe trials at Days 5 and 12, where platform is removed. A number of parameters including the latency to find the platform, time spent around the platform, path efficiency, % time spent in each quadrant is recorded, using automated tracking software (Ethovision). The type of exploration (direct, random, scan of the area) is also recorded. Motivation to swim and escape, as well as visual acuity is tested in case of doubt by making the platform visible (e.g. by putting a flag). The test takes place in a round arena of 120 cm diameter, filled with water (opacified with a white dye) at 23±1° C., virtually separated into four quadrants. Visual cues are located outside of the pool.

### Novel Object Recognition

Novel object recognition (NOR) is a well-established protocol to evaluate recognition memory in rodent models. Each mouse is handled for 5 minutes per day for at least 4 consecutive days to reduce animal's stress due to experimenter's presence/manipulation during test days. The animal is placed in an arena that contains two identical plastic objects (plastic building blocks, Falcon tubes, plastic cup) for 10 minutes while he can explore the objects, which constitutes the acquisition phase. During the acquisition period, time spent exploring and number of contacts with each object are recorded. At the end of acquisition, mouse is removed from the arena and placed back in its home cage. 24 hours (Test 1) and 7 days (Test 2) after the end of acquisition, mouse is placed back in the same arena for a duration of 10 minutes, with one of the acquisition objects being changed. The time of exploration and number of contacts with each object are recorded. The activity is recorded automatically using automated tracking software (Ethovision).

1-24. (canceled)

25. A compound of Formula (I), 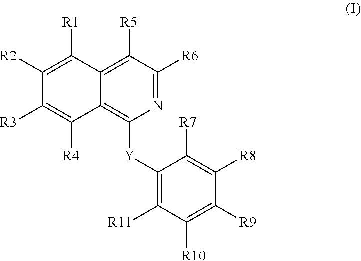
wherein Y is selected from NH and CH₂; R1 is selected from H, halogen and C₁-C₆ alkyl, optionally substituted with a group selected from halogen, OR¹² and NR¹³R¹⁴; R2 is selected from H, halogen, C₁-C₆ alkyl, optionally substituted with a group selected from halogen, OR¹² and NHR¹³, or C₂-C₆ alkenyl, or a group OR¹², NR¹³R¹⁴ or a cyano group or an optionally substituted heterocycle; R3 is selected from H, halogen, C₁-C₆ alky, optionally substituted with a group selected from halogen, OR¹² and NR¹³R¹⁴⁻ or C₂-C₆ alkenyl, or a group OR¹², or NHR¹³, or an optionally substituted heterocycle or a cyano group; R4 is selected from H, halogen C₁-C₆ alkyl, optionally substituted with a group selected from halogen, OR¹² and NHR¹³, or an optionally substituted heterocycle and a cyano group; R5 is selected from H, halogen, C₁-C₆ alkyl, optionally substituted with a group selected from halogen, OR¹² and NHR¹³, or a group OR¹², or NR¹³R¹⁴; R6 is selected from H, halogen, C₁-C₆ alkyl, optionally substituted with a group selected from halogen, OR¹² and NHR¹³, or a group OR¹², or NHR¹³; R7 and R8 are independently selected from H and halogen; R9 is selected from SO—C₁-C₆ alkyl, SO₂-C₁-C₆ alkyl, SO₂-C₃-C₆ cycloalkyl, or an optionally substituted heterocycle selected from optionally substituted imidazole, optionally substituted isoxazole, optionally substituted oxazole, optionally substituted pyridine, optionally substituted pyrimidine, optionally substituted pyrrolinone, and optionally substituted oxetane; R10 and R11 are independently selected from H and halogen; R12, R13, and R14 are independently selected from H, C(O)—C₁-C₆ alkyl and optionally substituted C₁-C₆ alkyl or C₃-C₆ cycloalkyl; any pharmaceutically acceptable salts, hydrates, solvates, or polymorphs, tautomers, optically active forms, enantiomeric mixtures thereof, and mixtures thereof, with the provision that said compound is not a compound selected from the following list:
N-[4-[5-Ethyl-3-(1-methylethyl)-1H-pyrazol-1-yl]phenyl]-1-isoquinolinamine, RN: 1101888-63-4;
N-[4-[5-Chloro-3-(trifluoromethyl)-1H-pyrazol-1-yl]phenyl]-1-isoquinolinamine, RN: 251657-99-5;
N-[4-[5-Ethyl-3-(3-pyridinyl)-1H-pyrazol-1-yl]phenyl]-1-isoquinolinamine, RN: 251658-04-5;
N-[4-[3,5-Bis(trifluoromethyl)-1H-pyrazol-1-yl]phenyl]-1-isoquinolinamine, RN: 251657-94-0;
N-[4-[3-(Tetrahydro-2-furanyl)-5-(trifluoromethyl)-1H-pyrazol-1-yl]phenyl]-1-isoquinolinamine, RN: 1101888-82-7;
N-[4-[3-(3-Pyridinyl)-5-(trifluoromethyl)-1H-pyrazol-1-yl]phenyl]-1-isoquinolinamine, RN: 251658-03-4;
3-Methyl-N-[4-(4-pyridinyl)phenyl]-1-isoquinolinamine, RN:1368370-93-7;
1-[[4-(4-Pyridinyl)phenyl]amino]-8-isoquinolinecarbonitrile, RN:1368269-53-7;
8-Methyl-N′-[4-(4-pyridinyl)phenyl]-1,5-isoquinolinediamine, RN:1369288-67-4;
5-Nitro-N-[4-(4-pyridinyl)phenyl]-1-isoquinolinamine, RN:1368370-43-7;
N-[4-(4-Pyridinyl)phenyl]-5-(trifluoromethyl)-1-isoquinolinamine, RN: 1367803-95-9; and
4-Bromo-N′-[4-(4-pyridinyl)phenyl]-1,7-isoquinolinediamine, 1369271-85-1.

26. The compound according to claim 25, wherein Y is CH₂.

27. The compound according to claim 25, wherein Y is NH.

28. The compound according to claim 25, wherein R1, R5, R4 and R6 are H.

29. The compound according to claim 25, wherein R2 is OR¹².

30. The compound according to claim 25, wherein R3 is OR¹².

31. The compound according to claim 25, wherein R3 is NHR¹³.

32. The compound according to claim 25, wherein R3 is halogen.

33. The compound according to claim 25, wherein R3 is H.

34. The compound according to claim 25, wherein R3 is optionally substituted C₁-C₆ alkyl.

35. The compound according to claim 25, wherein R10 and R11 are H.

36. The compound according to claim 25, wherein R10 is halogen.

37. The compound according to claim 25, wherein R11 is halogen.

38. The compound according to claim 25, wherein R7, R8, R10 and R11 are H.

39. The compound according to claim 25, wherein R9 is selected from SO₂-C₁-C₆ alkyl.

40. The compound according to claim 25, wherein R9 is SO₂-C₃-C₆ cycloalkyl.

41. A pharmaceutical composition comprising a compound of Formula (I), 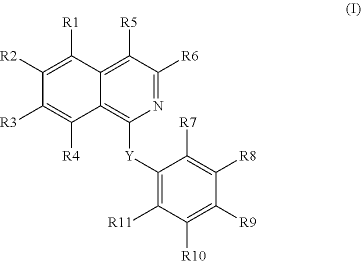
wherein Y is selected from NH and CH₂; R1 is selected from H, halogen and C₁-C₆ alkyl, optionally substituted with a group selected from halogen, OR¹² and NR¹³R¹⁴; R2 is selected from H, halogen, C₁-C₆ alkyl, optionally substituted with a group selected from halogen, OR¹² and NHR¹³, or C₂-C₆ alkenyl, or a group OR¹², NR¹³R¹⁴ or a cyano group or an optionally substituted heterocycle; R3 is selected from H, halogen, C₁-C₆ alky, optionally substituted with a group selected from halogen, OR¹² and NR¹³R¹⁴⁻ or C₂-C₆ alkenyl, or a group OR¹², or NHR¹³, or an optionally substituted heterocycle or a cyano group; R4 is selected from H, halogen C₁-C₆ alkyl, optionally substituted with a group selected from halogen, OR¹² and NHR¹³, or an optionally substituted heterocycle and a cyano group; R5 is selected from H, halogen, C₁-C₆ alkyl, optionally substituted with a group selected from halogen, OR¹² and NHR¹³, or a group OR¹², or NR¹³R¹⁴; R6 is selected from H, halogen, C₁-C₆ alkyl, optionally substituted with a group selected from halogen, OR¹² and NHR¹³, or a group OR¹², or NHR¹³; R7 and R8 are independently selected from H and halogen; R9 is selected from SO—C₁-C₆ alkyl, SO₂-C₁-C₆ alkyl, SO₂—C₃-C₆ cycloalkyl, or an optionally substituted heterocycle selected from optionally substituted imidazole, optionally substituted isoxazole, optionally substituted oxazole, optionally substituted pyridine, optionally substituted pyrimidine, optionally substituted pyrrolinone, and optionally substituted oxetane; R10 and R11 are independently selected from H and halogen; R12, R13, and R14 are independently selected from H, C(O)— C₁-C₆ alkyl and optionally substituted C₁-C₆ alkyl or C₃-C₆ cycloalkyl; and a pharmaceutically acceptable carrier, diluent or excipient thereof, with the proviso that the compound is not a compound selected from the following list:
3-Methyl-N-[4-(4-pyridinyl)phenyl]-1-isoquinolinamine, RN:1368370-93-7;
1-[[4-(4-Pyridinyl)phenyl]amino]-8-isoquinolinecarbonitrile, RN:1368269-53-7;
8-Methyl-N¹-[4-(4-pyridinyl)phenyl]-1,5-isoquinolinediamine, RN:1369288-67-4;
5-Nitro-N-[4-(4-pyridinyl)phenyl]-1-isoquinolinamine, RN:1368370-43-7;
N-[4-(4-Pyridinyl)phenyl]-5-(trifluoromethyl)-1-isoquinolinamine, RN: 1367803-95-9; and
4-Bromo-N¹-[4-(4-pyridinyl)phenyl]-1,7-isoquinolinediamine, 1369271-85-1.

42. A method of increasing intracerebral glucose and/or lactate levels in a subject, enhancing cognitive and memory functions in a subject, preventing or treating a disorder or a disease associated with an abnormally low energy metabolism or in the central nervous system and/or a neurological disorder or for the treatment or stabilizing a neurological disorder with brain hypometabolism or associated symptoms in a subject, said method comprising administering in a subject in need thereof a therapeutically effective amount of a compound of Formula (I), 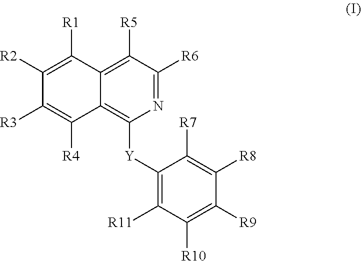
wherein Y is selected from NH and CH₂; R1 is selected from H, halogen and C₁-C₆ alkyl, optionally substituted with a group selected from halogen, OR¹² and NR¹³R¹⁴; R2 is selected from H, halogen, C₁-C₆ alkyl, optionally substituted with a group selected from halogen, OR¹² and NHR¹³, or C₂-C₆ alkenyl, or a group OR¹², NR¹³R¹⁴ or a cyano group or an optionally substituted heterocycle; R3 is selected from H, halogen, C₁-C₆ alky, optionally substituted with a group selected from halogen, OR¹² and NR¹³R¹⁴⁻ or C₂-C₆ alkenyl, or a group OR¹², or NHR¹³, or an optionally substituted heterocycle or a cyano group; R4 is selected from H, halogen C₁-C₆ alkyl, optionally substituted with a group selected from halogen, OR¹² and NHR¹³, or an optionally substituted heterocycle and a cyano group; R5 is selected from H, halogen, C₁-C₆ alkyl, optionally substituted with a group selected from halogen, OR¹² and NHR¹³, or a group OR¹², or NR¹³R¹⁴; R6 is selected from H, halogen, C₁-C₆ alkyl, optionally substituted with a group selected from halogen, OR¹² and NHR¹³, or a group OR¹², or NHR¹³; R7 and R8 are independently selected from H and halogen; R9 is selected from SO—C₁-C₆ alkyl, SO₂-C₁-C₆ alkyl, SO₂—C₃-C₆ cycloalkyl, or an optionally substituted heterocycle selected from optionally substituted imidazole, optionally substituted isoxazole, optionally substituted oxazole, optionally substituted pyridine, optionally substituted pyrimidine, optionally substituted pyrrolinone, and optionally substituted oxetane; R10 and R11 are independently selected from H and halogen; R12, R13, and R14 are independently selected from H, C(O)— C₁-C₆ alkyl and optionally substituted C₁-C₆ alkyl or C₃-C₆ cycloalkyl; a tautomer, a geometrical isomer, an optically active form, an enantiomeric mixture, a pharmaceutically acceptable salt, a pharmaceutically active derivative thereof or a mixture thereof.

43. The method according to claim 42, said method increasing the intracerebral glucose and/or lactate levels in a subject and said method comprising administering to a subject in need thereof an effective amount of said compound or pharmaceutically acceptable salts, hydrates, solvates, or polymorphs, tautomers, optically active forms, enantiomeric mixtures thereof and pharmaceutically active derivative and mixtures thereof to induce increased intracerebral glucose and/or lactate levels.

44. The method according to claim 42, said method enhancing cognitive and memory functions in a subject and said method comprising administering to said subject an effective amount of said compound or pharmaceutically acceptable salts, hydrates, solvates, or polymorphs, tautomers, optically active forms, enantiomeric mixtures thereof and pharmaceutically active derivative and mixtures thereof.

45. The method according to claim 42, wherein said compound is selected from the following list:
1-[4-[(6,7-dimethoxy-1-isoquinolyl)methyl]phenyl]pyrrolidin-2-one;
6,7-dimethoxy-N-(4-methylsulfonylphenyl)isoquinolin-1-amine;
6-methoxy-1-(4-methylsulfonylanilino)isoquinolin-7-ol;
7-methoxy-1-(4-methylsulfonylanilino)isoquinolin-6-ol;
1-(4-methylsulfonylanilino)isoquinoline-6,7-diol;
6-ethoxy-1-(4-methylsulfonylanilino)isoquinolin-7-ol;
7-ethoxy-1-(4-methylsulfonylanilino)isoquinolin-6-ol;
6,7-diethoxy-N-(4-methylsulfonylphenyl)isoquinolin-1-amine;
1-[4-[(6,7-dimethoxy-1-isoquinolyl)amino]phenyl]pyrrolidin-2-one;
N-(4-methylsulfonylphenyl)-6-vinyloxy-isoquinolin-1-amine;
6-isopropoxy-N-(4-methylsulfonylphenyl)isoquinolin-1-amine;
N-(4-methylsulfonylphenyl)-6-pyrimidin-2-yl-isoquinolin-1-amine;
N-(4-methylsulfonylphenyl)-6-pyrimidin-2-yl-isoquinolin-1-amine;
N-[1-(4-methylsulfonylanilino)-7-isoquinolyl]acetamide;
N7-ethyl-N1-(4-methylsulfonylphenyl)isoquinoline-1,7-diamine;
6-methoxy-N-(4-methylsulfonylphenyl)-7-vinyl-isoquinolin-1-amine;
7-ethyl-6-methoxy-N-(4-methylsulfonylphenyl)isoquinolin-1-amine;
7-bromo-1-(4-methylsulfonylanilino)isoquinolin-6-ol;
N7-benzyl-6-methoxy-N1-(4-methylsulfonylphenyl)isoquinoline-1,7-diamine;
N7-(cyclopropylmethyl)-6-methoxy-N1-(4-methylsulfonylphenyl)isoquinoline-1,7-diamine;
6-methoxy-N1-(4-methylsulfonylphenyl)-N7-propyl-isoquinoline-1,7-diamine;
N6-(cyclopropylmethyl)-7-methoxy-N1-(4-methylsulfonylphenyl)isoquinoline-1,6-diamine;
6-(azetidin-1-yl)-7-methoxy-N-(4-methylsulfonylphenyl)isoquinolin-1-amine;
6-ethyl-1-[(4-methanesulfonylphenyl)amino]isoquinolin-7-ol;
N-[4-(ethanesulfonyl)phenyl]-6,7-diethoxyisoquinolin-1-amine;
6,7-diethoxy-N-[4-(oxetan-3-yl)phenyl]isoquinolin-1-amine;
6,7-diethoxy-N-[4-(1,2-oxazol-3-yl)phenyl]isoquinolin-1-amine;
6,7-diethoxy-N-[4-(1,2-oxazol-5-yl)phenyl]isoquinolin-1-amine;
6,7-diethoxy-N-(3-fluoro-4-methanesulfonylphenyl)isoquinolin-1-amine;
6,7-diethoxy-N-(2-fluoro-4-methanesulfonylphenyl)isoquinolin-1-amine;
N-[4-(cyclopropanesulfonyl)phenyl]-6,7-diethoxyisoquinolin-1-amine;
6-ethoxy-7-(2-fluoroethoxy)-N-(4-methanesulfonylphenyl)isoquinolin-1-amine;
6,7-bis(2-fluoroethoxy)-N-(4-methanesulfonylphenyl)isoquinolin-1-amine;
1-[(4-methanesulfonylphenyl)methyl]-6-(propan-2-yloxy)isoquinoline;
6-[(4-fluorophenyl)methoxy]-N-(4-methanesulfonylphenyl)isoquinolin-1-amine;
6-ethoxy-N-(4-methanesulfonylphenyl)-7-propylisoquinolin-1-amine; and
any pharmaceutically acceptable salts, hydrates, solvates, or polymorphs, tautomers, optically active forms, enantiomeric mixtures thereof, and mixtures thereof.
